(19) United States
(12) Patent Application Publication
(10) Pub. No.: US 2024/0358727 A1
(43) Pub. Date: Oct. 31, 2024

(54) MICROSCALE THERMOPHORESIS TO EVALUATE CHANNEL LIPID INTERACTIONS AND METHODS OF TREATING NAV CHANNELS RELATED DISEASES

(71) Applicant: Northeastern University, Boston, MA (US)

(72) Inventors: Kirin D. Gada, Watertown, MA (US); Leigh D. Plant, Boston, MA (US)

(21) Appl. No.: 18/635,427

(22) Filed: Apr. 15, 2024

Related U.S. Application Data
(60) Provisional application No. 63/617,884, filed on Jan. 5, 2024.
(60) Provisional application No. 63/458,987, filed on Apr. 13, 2023.

(51) Int. Cl. A61K 31/683; A61K 31/10; A61K 31/132; A61K 31/137; A61K 31/235; A61K 31/265; A61K 31/27; A61K 31/365; A61K 31/58; A61K 31/685; A61K 31/7036; A61P 9/06; G01N 33/50

(52) U.S. Cl. CPC A61K 31/683; A61K 31/10; A61K 31/132; A61K 31/137; A61K 31/235; A61K 31/265; A61K 31/27; A61K 31/365; A61K 31/58; A61K 31/685; A61K 31/7036; A61P 9/06; G01N 33/502

(57) ABSTRACT

Voltage-gated sodium (NaV) channels are densely expressed in most excitable cells and activate in response to depolarization, causing a rapid influx of Na+ ions that initiates the action potential. The voltage-dependent activation of NaV channels is followed almost instantaneously by fast inactivation, setting the refractory period of excitable tissues. The gating cycle of NaV channels is subject to tight regulation, with perturbations leading to a range of pathophysiological states. The gating properties of most ion channels are regulated by the membrane phospholipid, phosphatidylinositol (4,5) bisphosphate (PI(4,5)P2). However, it is not known whether PI(4,5)P2 modulates the activity of NaV channels. Here, we utilize optogenetics to activate specific, membrane-associated phosphoinositide (PI)-phosphatases that dephosphorylate PI(4,5)P2 while simultaneously recording NaV1.4 channel currents. We show that dephosphorylating PI(4,5)P2 left-shifts the voltage-dependent gating of NaV1.4 to more hyperpolarized membrane potentials, augments the late current that persists after fast inactivation, and speeds the rate at which channels recover from fast inactivation. These effects are opposed by exogenous diC8PI(4,5)P2. We provide evidence that PI(4,5)P2 is a negative regulator that tunes the gating behavior of NaV1.4 channels.

### RELATED APPLICATIONS

This application claims the benefit of priority to U.S. Provisional Patent Application Ser. No. 63/617,884, filed Jan. 5, 2024; and U.S. Provisional Patent Application Ser. No. 63/458,987, filed Apr. 13, 2023.

### GOVERNMENT SUPPORT

This invention was made with government support under Grant R01HL144615 awarded by the National Institutes of Health. The government has certain rights in the invention.

### BACKGROUND

Several diseases are caused by mutations in the genes that encode voltage-gated sodium channels. Some of these diseases, such as specific cardiac arrhythmia's (like Brugada Syndrome and long QT type 3), and specific epilepsies can be fatal. Others, such as muscle paralytic or weakening disorders (like paramyotonia congenita; PMC), have a profound negative impact on the quality of a patient's life. In addition to mutations, these disorders can arise from adverse drug effects, or dysregulation of voltage-gated sodium channels (Nav). Despite progress in unraveling the causes of how the sodium channels have altered, detrimental function many open questions remain unanswered hampering our ability to find cures or better drug treatments to manage these disorders. Therefore, there is a critical need to novel methods to treat diseases caused by mutations in the genes that encode voltage-gated sodium channels.

### SUMMARY

Disclosed are methods of treating or preventing a cardiovascular disease, a pain syndrome, epilepsy, or a skeletal muscle disease. The method may comprise administering to a subject in need thereof an effective amount of a compound. In some embodiments, the compound increases the interaction between NaV1.5 voltage-gated sodium channel or a NaV1.4 voltage-gated sodium channel and Phosphatidylinositol 4,5-bisphosphate (PIP2). In some embodiments, the compound is an analogue of PIP2, such as diC8-PIP2. In some embodiments, the compound is PIP2. In some embodiments, the compound increases endogenous PIP2.

In some embodiments, the compound increases endogenous PIP2; and the compound is an inhibitor a Gaq-coupled AT1 receptor, such as 3 losartan, Exp 3174, telmisartan, irbesartan, candesartan, valsartan, eprosartan, azilsartan, saprisartan or olmesartan. In some embodiments, the compound increases endogenous PIP2; and the compound is an inhibitor of phospholipase C (PLCβ), such as U73122, phenylmethylsulfonyl fluoride, manoalide, D609, ET-18-OCH3, compound 48/80 trihydrochloride, spermine tetrahydrochloride, neomycin sulfate, NCDC, or thielavin B. In some embodiments, the compound increases endogenous PIP2; and the compound inhibits PIP2 hydrolysis or inhibits PIP2 dephosphorylation.

In some embodiments, a cardiovascular disease is treated or prevented; and the cardiovascular disease is arrhythmias, long QT syndrome (LQT3), Brugada syndrome (BrS), cardiac conduction defects, atrial fibrillation, and dilated cardiomyopathy, sudden infant death syndrome (SIDS), or sudden cardiac death in adults. In some embodiments, a pain syndrome is treated or prevented; and the pain syndrome is a chronic pain syndrome, fibromyalgia, or neuropathic pain. In some embodiments, epilepsy is treated or prevented; and the epilepsy is an idiopathic generalized epilepsy, idiopathic partial epilepsy, symptomatic generalized epilepsy or symptomatic partial epilepsy. In some embodiments, the symptomatic partial epilepsy is temporal lobe epilepsy. In some embodiments, the subject suffers from a traumatic brain injury, hypoxic brain injury, brain infection, stroke, or genetic syndrome. In some embodiments, the subject suffers from a brain infection; and the brain infection is encephalitis, meningitis, mesial temporal sclerosis, or a cerebral tumor. In some embodiments, the epilepsy is at least partially induced by the traumatic brain injury, hypoxic brain injury, brain infection, stroke, or genetic syndrome. In some embodiments, the epilepsy is a TBI-induced epilepsy. In some embodiments, a skeletal muscle disease is a periodic paralysis (PP), a nondystrophic myotonia (NDM), and a ryanodinopathy. In some embodiments, the periodic paralysis (PP) or the nondystrophic myotonia (NDM) is myotonia congenita, paramyotonia congenita (PMC), or potassium-aggravated myotonia (PAM), hyper and hypokalemic periodic paralysis (hyperPP and hypoPP), or Andersen-Tawil syndrome (ATS). In some embodiments, the ryanodinopathy is malignant hyperthermia (MH), central core disease (CCD), multi-minicore disease (MmD), or centronuclear myopathy (CNM).

In another aspect, the present invention provides methods of identifying a compound that modulates the interaction between a NaV1.5 voltage-gated sodium channel or a NaV1.4 voltage-gated sodium channel and PIP2. The method may comprise contacting cells expressing a tagged NaV1.5 channel (or a variant thereof), or a tagged NaV1.4 channel (or a variant thereof) with a candidate agent and PIP2 or a PIP2 analog (such as diC8PI(4,5)P2). In other embodiments, the method comprises contacting cell lysate of cells expressing a tagged NaV1.5 channel (or a variant thereof), or a tagged NaV1.4 channel (or a variant thereof) with a candidate agent and PIP2 or a PIP2 analog (such as diC8PI(4,5)P2). The method may further comprise detecting the affinity of PIP2 or the PIP2 analog to the tagged NaV1.5 channel or the tagged NaV1.4 channel. The method may further comprise comparing the affinity in the presence of the candidate agent with the affinity in the absence of the candidate agent; wherein a change in affinity in the presence of the candidate agent is indicative of modulation of the interaction between NaV1.5 or NaV1.4 and PIP2.

In some embodiments, the tagged NaV1.5 channel, the tagged NaV1.4 channel, or a variant thereof is isolated from the cell lysate before incubating with the candidate agent and PIP2 or the PIP2 analog. In some embodiments, the tagged NaV1.5 channel, the tagged NaV1.4 channel, or a variant thereof is tagged with a fluorescent protein. In some embodiments, the fluorescent protein is a green fluorescent protein (GFP), a yellow fluorescent protein (YFP), a red fluorescent protein (RFP), or a monomeric teal fluorescent protein (mTFP). In some embodiments, the affinity is detected via an instrument detecting the tagged protein. In some embodiments, the candidate agent is a small molecule or a peptide.

### BRIEF DESCRIPTION OF THE DRAWINGS

FIG. 1 shows a schematic summarizing our proposal that Gaq signaling in cardiomyocytes modules I_{Na} and I_{LATE}. Left, Gαq-coupled receptors such as AT1, ET_{A} and the heterologous M3q DREADD signal through Gαq to activate PLCβ which hydrolyzes PIP₂. Our experiments are designed to test that PIP₂ interactions modulate Nav1.5 channel activity in cardiomyocytes. Right, example whole-cell data show, in M3q iPS-CMs, that 100 nM CNO evokes a leftward shift in the current-voltage (I-V) relationship and increased peak I_{Na} and I_{LATE}. These effects are not blocked by staurosporine or BAPTA, indicating that the effector is not the downstream signals of DAG activation of PKC or IP3-mediated increase in intracellular Ca²⁺. In contrast, the effects are abolished by the PLC inhibitor U73122 or including soluble diC8-PIP₂ in the patch pipette.

FIGS. 2A-2D show that hydrolysis of PIP₂ modulates voltage-dependent gating of I_{Na} in iPS-CMs. FIG. 2A shows schematic showing that the PIP₂ biosensor iRFP-PH_{PLCδ} disengages from the membrane following activation of M3q. FIG. 2B shows TIRF data showing rate of disengagement of iRFP-PH_{PLCδ} from the membrane of M3q-cells treated with 100 nM CNO. Control is CNO application to cells with no M3q. Mean±SD % fluorescence change for 20 cells per group; an example photobleaching montage is inset. Note that the time course is concordant with increase in I_{LATE} shown in FIG. 3C. FIG. 2C shows normalized conductance (G/Gmax) and steady state inactivation (SSI) curves from M3q-iPS-CMs before and after application of 100 nM CNO. FIG. 2D shows summary of the mean±sem V½_{ACT} and V ½_{SSI} data for 8 cells per experiment, **P<0.01 paired t-test. Leftwards shifts in each parameter are precluded by U73122 or by 200 μM diC8-PIP₂ in the pipette but are not sensitive to staurosporine or BAPTA. The cells were perfused with an extracellular solution containing (in mM): 130 NaCl, 5 CsCl, 1.2 MgCl₂, 1.5 CaCl₂), 8 Glucose, 10 HEPES (pH7.4). 10 mM nitrendipine was added to the recording solution to block Ca²⁺ flux through Cav channels. Patch pipettes with a resistance of 2.5-4 MQ are filled with a solution containing (in mM): 60 CsCl, 80 CsF, 2 MgCl₂, 10 EGTA, 5 Na₂ATP, 5 HEPES (pH 7.4). Currents were low pass (Bessel) filtered at 9 kHz and digitized at 50 kHz and were collected from cells with a series resistance <15 MQ; this was compensated by 70%.

FIGS. 3A-3C show that hydrolysis of PIP₂ modulates I_{LATE} in M3q iPS-CMs. FIG. 3A shows example records showing elevated I_{LATE} in response to 100 nM CNO. Data are obtained using the recording conditions described in FIG. 2. FIG. 3B shows summary of the mean±sem change in % I_{LATE} for 8 cells per experiment, **P<0.01 paired t-test. Increased I_{LATE} is precluded by U73122 or including 200 μM diC8-PIP₂ in the pipette but are not sensitive to staurosporine or BAPTA. FIG. 3C shows mean time course for the increase in I_{LATE}, the arrow indicated application of 100 nM CNO. Note that the time course is concordant with the uncoupling of the PIP₂ biosensor shown in FIG. 2B.

FIGS. 4A-4B shows that Ang II modulates I_{Na} and I_{LATE} in iPS-CMs. FIG. 4A left shows I_{Na} evoked by a −45 mV test pulse is elevated by 100 nM Ang II. FIG. 4A right shows normalized conductance (G/Gmax) and steady state inactivation (SSI) curves from iPS-CMs before and after application of 100 nM Ang II (blue). The data are mean±sem for 4 cells per experiment. Note leftwards shifts in each curve. FIG. 4B Left shows example records showing elevated I_{LATE} in response to 100 nM Ang II. FIG. 4B Right shows time course for 4 cells showing the increase in I_{LATE}, the arrow indicates Ang II application.

FIGS. 5A-5D shows that DiC8-PIP₂ rescues I_{LATE} in Nav1.5-R1644C. FIG. 5A shows schematic of optogenetic dephosphorylation of PIP₂ by CRY2-5P_{OCRL}. FIG. 5B Left shows example trace showing that Nav1.5-R1644C has elevated I_{LATE} (green), this is further augmented by dephosphorylation of PIP₂ (blue light, BL). FIG. 5B Right upper shows summary data for the increase in I_{LATE}. FIG. 5B Lower shows elevated baseline I_{LATE} is decreased by 200 μM diC8-PIP₂ in the pipette and the increase with PIP₂ dephosphorylation is opposed. FIG. 5C shows that I_{LATE} in WT-Nav1.5 is increased when PIP₂ is dephosphorylated, this is opposed by diC8-PIP₂. FIG. 5D shows that I_{LATE} rises more rapidly for R1644C than WT-Nav1.5. This is opposed when by 200 μM diC8-PIP₂ in the pipette. Comparisons are Students t-test, *** P<0.01, ****P0.001 for 4-6 cells per group.

FIGS. 6A-6B shows that Nav1.5-R1644C has a reduced binding affinity for diC8-PIP₂. FIG. 6A Left shows example microscale thermophoresis (MST) fluorescence response curves for GFP-tagged PH_{PLCδ} (PIP₂ biosensor) showing the transition from unbound to bound as the concentration of diC8-PIP₂ is increased. Right, Normalized response curves: The binding affinity (Kd) of GFP-PH_{PLCδ} is 130±15 nM, whereas free GFP does not bind diC8-PIP₂. FIG. 6B shows that Nav1.5 WT has a Kd for diC8-PIP₂ of 45±10 nM, this is increased to 445±25 nM in Nav1.5-R1644C. Data are the means of 3-5 separate replicates per condition.

FIG. 7 shows that MD simulation results for rat Nav1.5. The minimum distances between gate residues on the opposite domains as function of simulation time. The minimum distances of A414-I1472 and L941-I1773 are 0.755 nm and 0.815 nm, respectively, in the cryo-EM structure PDB:6UZ3 (dashed lines).

FIGS. 8A-8B shows molecular docking predicted potential PIP₂ binding sites on hNav1.5 channel. FIG. 8A shows top view model of hNav1.5 bound with four PIP₂ molecules. FIG. 8B shows 2D interaction plot between the residues (near VSD of DIV) and diC1-PIP₂.

FIGS. 9A-9C show Nav1.5 cryoEM structure. RatNav1.5 was purified in GDN and imaged using a Talos (200 kV)+K3 cryoEM system (UMass facility). Data are magnified 100K, pixel size 0.82 Apix, total dose 49 e⁻/A². FIG. 9A shows that 2D classification was performed on data from 5184 micrographs. FIG. 9B show different views. Bi, sideview with detergent; Bii sideview no GDN; Biii sideview model fit to PBD 6UZ3; Biv, top-down view. FIG. 9C shows fits show resolution is 3.71 Å.

FIGS. 10A-10D show the effect of dephosphorylation of PI(4,5)P₂ on the activity of Naᵥ1.5 channels.

FIGS. 11A-11C show the effect of dephosphorylating of PI(4,5)P₂ on the voltage-dependence of steady-state inactivation (SSI).

FIGS. 12A-12C show that dephosphorylation of PI(4,5)P₂ has an effect on the I_{LATE}.

FIGS. 13A-13D show the effect of dephosphorylating of PI(4,5)P₂ on V½_{SSI}.

FIGS. 14A-14C show that in both Naᵥ1.4 and Naᵥ1.5, changes in either V½_{ACT} or both the V½_{ACT} and V½_{SSI} produced an increase in the window current.

FIGS. 15A-15E show dephosphorylation of PI(4,5)P₂ at the 5-position has an effect on the I_{LATE}.

FIGS. 16A-16D show that PI(4,5)P₂ enriched environment impacts Naᵥ1.5 channel activity.

FIGS. 17A-17C show the effect diC8PI(4,5)P₂ enrichment the voltage-dependence of SSI.

FIGS. 18A-18C show that diC8PI(4,5)P₂ opposed the effect of the dephosphorylation.

FIGS. 19A-19D show that PI(4,5)P₂ enriched environment impacts Naᵥ1.5 channel activity.

FIGS. 20A-20C show the effect of diC8PI(4,5)P₂ enrichment on the voltage dependence of activation.

FIGS. 21A-21C show the effect diC8PI(4,5)P₂ enrichment the voltage-dependence of SSI. Voltage-dependence of SSI.

FIGS. 22A-22D show that dephosphorylation application diC8PI(4,5)P₂ oppose the effect of CRY2-5P_{OCRL} dephosphorylation on I_{LATE} and rate of fast inactivation.

FIGS. 23A-23B show the relationship between Nav1.5 and PI(4,5)P₂ through a protein-ligand lens.

FIGS. 24A-24D show that 1, Dephosphorylation of PI(4,5)P₂ augments Nav1.4 currents. HEK293T cells were transfected to express CRY2-tagged pseudojanin (CRY2-PJ) and CIBN-CAAX with the PI(4,5)P₂ biosensor, iRFP-PH_{PLCδ} for TIRFM imaging or rNav1.4 and Navβ1 for whole cell patch clamp studies. CRY2-PJ is the 5-phosphatase inositol polyphosphate 5-phosphatase E (INPP5E) fused to the 4-phosphatase Sacd. TIRF data are mean, normalized fluorescence values±s.e.m. for 30 membrane-delimited regions of interest identified from 8 cells. Patch-clamp data are paired before and after at least 3-mins of blue-light (BL) photoactivation for 18 cells per group obtained from 3 independent biological replicates of the experiment. Statistical significance was determined using a Student's paired t-test. FIG. 24A shows schematic showing that CRY2-PJ is targeted to the membrane by blue light (BL) where it dephosphorylates PI(4,5)P₂ to PI(4)P and, subsequently PI. FIG. 24B shows a montage of TIRF fluorescent micrographs showing decoupling of the PI(4,5)P₂ biosensor iRFP-PH_{PLCδ1} from the membrane in response to photoactivation of CRY2-PJ. The images are every 40 and the scale bar=10 μm. FIG. 24C Left shows a representative time course of normalized iRFP-PH_{PLCδ} fluorescence showing loss of the biosensor from the membrane following BL-activation of CRY2-PJ. Right, Summary data showing that >80% of iRFP-PH_{PLCδ} is decoupled from the membrane 200 s by BL-activation of CRY2-PJ. FIG. 24D Left shows representative traces of Nav1.4 currents evoked by a test pulse to −40 mV, before (dark) and after (light gray) dephosphorylation of PI(4,5)P₂ by CRY2-PJ. Right, mean current-voltage relationships for Nav1.4 channels recorded before (dark) and after (light gray) dephosphorylation of PI(4,5)P₂ by CRY2-PJ.

FIGS. 25A-25C show that PI(4,5)P₂ tunes the voltage-dependence of Nav1.4 channels. Whole-cell currents for rNav1.4+Navβ1 were recorded as per FIG. 24 with 200 μM diC8PI(4,5)P₂ in the patch pipette where indicated. The voltage-dependence of activation (G/Gmax) and steady state inactivation (1/Imax) were determined as described in the Materials and Methods using fits with Boltzmann functions for data obtained before and at least 3-mins after blue-light (BL) activation of CRY2-PJ. The data are for 18 cells per group obtained from 3 independent biological replicates of the experiment. Statistical significance was determined using a Student's paired t-test. FIG. 25A Left shows Mean activation (circle) and SSI (square) curves for Nav1.4 before (black) and after blue light photoactivation of CRY2-PJ (BL, blue). The window current is inset. Right, Summary of the change in the V½_{act} and V½_{SSI} following photoactivation of CRY2-PJ (BL). FIG. 25B Left shows mean activation (circle) and SSI (square) curves for Nav1.4 in the presence of 200 μM diC8PI(4,5)P₂ before, and after blue light photoactivation of CRY2-PJ (BL). The window current is inset. Right, Summary of the change in the V½_{ACT} and V½_{SSI} following photoactivation of CRY2-PJ (BL). FIG. 25C Left shows the activation and SSI curves from above to show the full excursion in the activation and SSI curves for cells with high levels of diC8PI(4,5)P₂, and fully dephosphorylated endogenous PI(4,5)P₂. The control condition is black, from (FIG. 25A). The window current is inset. Right, Unpaired data to show the differences in V½_{ACT} and V½_{SSI} described by the Boltzmann functions.

FIGS. 26A-26D show that Nav1.4 currents are augmented by CRY2-5P_{OCRL} dephosphorylation of PI(4,5)P₂. HEK293T cells were transfected to express CRY2-tagged 5-phosphatase OCRL (CRY2-5P_{OCRL}), and CIBN-CAAX with the PI(4,5)P₂ biosensor, iRFP-PH_{PLCδ} for TIRFM imaging or rNav1.4 and Navβ1 for whole cell patch clamp studies. TIRF data are from 30 membrane-delimited regions from 8 cells. Patch-clamp data are paired before and after at least 3-mins of blue-light (BL) photoactivation for 12 cells per group obtained from 3 independent biological replicates of the experiment. Statistical significance was determined using a Student's paired t-test. FIG. 26A shows schematic showing that CRY2-5P_{OCRL} is targeted to the membrane by blue light (BL) where it dephosphorylates PI(4,5)P₂ to PI(4)P. FIG. 26B shows a montage of TTRF fluorescent micrographs showing decoupling of the PI(4,5)P₂ biosensor iRFP-PH_{PLCδ1} from the membrane following photoactivation of CRY2-5P_{OCRL}. The images are every 40 s; scale bar=10 μm. FIG. 26C Left shows a representative time course of normalized iRFP-PH_{PLCδ} fluorescence showing the decrease in membrane association of the biosensor following blue-light activation of CRY2-5P_{OCRL}. Right, Summary data showing that >80% of iRFP-PH_{PLCδ} is decoupled from the membrane 200 s after blue-light activation of CRY2-5P_{OCRL}. FIG. 26D Left shows representative traces of Nav1.4 currents evoked by a test pulse to −40 mV, before (black) and after (gray) dephosphorylation of PI(4,5)P₂ by CRY2-5P_{OCRL}. Right, Mean current-voltage relationships for Nav1.4 channels recorded before (black) and after (gray) dephosphorylation of PI(4,5)P₂ by CRY2-5P_{OCRL}.

FIG. 27A-27C show that 5-dephosphorylation of PI(4,5)P₂ tunes the voltage-dependence of Nav1.4 channels. Whole-cell currents for rNav1.4+Navβ1 were recorded as per FIG. 26 with 200 μM diC8PI(4,5)P₂ in the patch pipette where indicated. The voltage-dependence of activation (G/Gmax) and steady state inactivation (1/Imax) were determined as described in the Materials and Methods using fits with Boltzmann functions for data obtained before and at least 3-mins after activation of CRY2-5P_{OCRL} by blue light (BL). The data are from 12 cells per group obtained from 3 independent biological replicates of the experiment. Statistical significance was determined using a Student's paired t-test. FIG. 27A Left shows mean activation (circle) and SSI (square) curves for Nav1.4 before (black) and after blue light photoactivation of CRY2-5P_{OCRL} (BL, gray). The window current is inset. Right, Summary of the change in the V½_{ACT} and V½_{SSI} following photoactivation of CRY2-5P_{OCRL} (BL). FIG. 27B Left shows mean activation (circle) and SSI (square) curves for Nav1.4 in the presence of 200 μM diC8PI(4,5)P₂ before, and after blue light photoactivation of CRY2-5P_{OCRL} (BL). The window current is inset. Right, Summary of the change in the V½_{act} and V½_{SSI} following photoactivation of CRY2-5P_{OCRL} (BL). FIG. 27C Left shows the activation and SSI curves from above to show the full excursion in the activation and SSI curves for cells with high levels of diC8PI(4,5)P₂, and fully dephosphorylated endogenous PI(4,5)P₂. The control condition is black, from (FIG. 27A). The window current is inset. Right, Unpaired data to show the differences in V½_{act} and V½_{SSI} described by the Boltzmann functions.

FIGS. 28A-28D show that dephosphorylating PI(4,5)P₂ speeds the recovery of Nav1.4 channels from fast inactivation. Recovery from fast inactivation at −30 mV in HEK293T cells transfected with rNav1.4, Navβ1, CIBN-CAAX and CRY2-PJ (FIGS. 28A, 28B) or CRY2-5P_{OCRL} (FIGS. 28C, 28D) were tested in whole-cell patch clamp experiments according to the protocol described in the Material and Methods. Normalized data are fit with an exponential function to get the time-constant, tau. The data are paired before and after at least 3-mins of blue-light (BL) photoactivation for 8 cells per group obtained from 2 independent biological replicates of the experiment. Statistical significance was determined using a Student's paired t-test. FIG. 28A left shows time-course of recovery from fast inactivation in response to a paired-pulse protocol before (black) and after activation of CRY2-PJ with blue light (BL). Right, Paired tau values. FIG. 28B left shows photoactivation of CRY2-PJ did not speed tau when 200 μM diC8PI(4,5)P₂ was included in the patch-pipette. FIG. 28C left shows Tau was also speeded following activation of CRY2-5P_{OCRL} with blue light (BL). Right, Paired tau values. FIG. 28D left shows photoactivation of CRY2-5P_{OCRL} did not speed tau when 200 μM diC8PI(4,5)P₂ was included in the patch-pipette.

FIGS. 29A-29E show that dephosphorylating PI(4,5)P₂ augments the late sodium current. HEK293T cells transfected with rNav1.4, Navβ1, CIBN-CAAX and CRY2-PJ (FIGS. 29A-29C) or CRY2-5P_{OCRL} (FIGS. 29D, 29E) and were studied by a 400 ms pulse to −20 mV before and at least 3-mins after illumination with blue light (BL). I_{LATE} is expressed as a percent of I_{PEAK}. The time course for fast inactivation is determined by an exponential fit and is expressed as tau. The data are for 10-15 cells per group obtained from 3 independent biological replicates of the experiment. Statistical significance was determined using a Student's paired t-test. FIG. 29A shows representative late current traces before (black) and after BL-activation of CRY2-PJ (gray). The scale bars are 50 pA and 50 ms. Inset, a zoomed-in view of the late current. FIG. 29B shows I_{LATE} is increased 8-fold by BL activation of CRY2-PJ; the effect is precluded when 200 μM diC8PI(4,5)P₂ is included in the patch pipette. FIG. 29C shows that Tau is slowed by activation of CRY2-PJ in control cells but does not change with 200 μM diC8PI(4,5)P₂ in the patch pipette. FIG. 29D shows that I_{LATE} increases 8-fold following BL activation of CRY2-5P_{OCRL} (blue) in control cells but not when 200 μM diC8PI(4,5)P₂ (green) is included in the patch pipette. FIG. 29E shows that Tau is slowed by activation of CRY2-5P_{OCRL} (blue) in control cells but does not change with 200 μM diC8PI(4,5)P₂ in the patch pipette.

FIGS. 30A-30C show that PI(4,5)P₂-dependent tuning of V½-activation requires the fast inactivation process. Xenopus oocytes were injected with cRNA for Nav1.4-WCW, Navβ1, CIBN-CAAX and the CRY2-fused phosphatases indicated and studied 48 hours later by two-electrode voltage clamp experiments. Currents were evoked by a step protocol before (black), and 3 mins after sustained photoactivation with blue-light (BL). The voltage-dependence of activation was determined as described in the Materials and Methods using fits with Boltzmann functions. Data are mean whole-oocyte currents±s.e.m. for 15-18 cells per condition obtained from 3 independent biological replicates of the experiment. FIG. 30A Left shows representative traces evoked by a step to −10 mV from oocytes expressing CRY2-5P_{OCRL} and CRY2-Sac2 before (black) and after (gray) photoactivation. Right, a normalized current-voltage relationship for the same experiment. FIG. 30B shows normalized G-V relationships for data in (FIG. 30A) show no change V½_{act} before (−23±4 mV) and after (−23±3 mV) blue light. FIG. 30C shows normalized G-V relationships for oocytes expressing CRY2-5P_{OCRL} show no change V½_{act} before (−22±5 mV) and after (−21±5 mV) blue light.

FIGS. 31A-31D show representative currents from Nav1.5-K1493E showing enhanced I_{LATE} that is further augmented by activation of CRY2-5ptase. FIG. 31B shows summary data for 6 cells, **P<0.01 paired t-test. FIG. 31C shows including 200 μM diC8-PI(4,5)P₂ opposes I_{LATE} and further changes due to activation of CRY2-5ptase. FIG. 31 shows summary data for 6 cells.

FIGS. 32A-32C show PI(4,5)P₂ opposed hypoxic inhibition of I_{K1}. FIG. 32A shows cartoon summarizing that acute hypoxia inhibits I_{K1} due to recruitment of SUMO1 to Lys49 of Kir2.1 in rat ventricular cardiomyocytes (RVCMs). FIG. 32B shows hypoxic inhibition of I_{K1} is opposed when diC8-PI(4,5)P₂ is included in the recording pipette. FIG. 32C Left shows example traces showing that including diC8-PI(4,5)P₂ opposes hypoxic-inhibition of I_{K1} in a concentration-dependent manner. The arrow indicates the change in current magnitude between exposure to ambient 02 and 2% 02 in the same cell. Right, Summary concentration-response curves from control RVCMs (red) and RVCMs expressing scrambled shRNA (gray). The hypoxic response is diminished in Kir2.1^{kd} (knockdown) RVCMs (cyan). Kir2.1 knockdown is by transduction with lentiviral particles carrying eGFP and shRNA targeting kcnj2. Kir2.1^{kd}-CMs were identified by expression of eGFP. Data are mean±s.d. for 8 cells per condition. Adapted from Xu et al., 2022.

### DETAILED DESCRIPTION

In the human heart, Nav1.5 voltage-gated sodium channels pass I_{Na}, a rapidly activating and inactivating Na⁺ current that determines the rise, duration, and timing of action potentials, as well as a small, persistent Na⁺ current, the late Na⁺ current (I_{LATE}), that contributes to maintaining the action potential plateau. Therefore, all aspects of Nav1.5 channel gating are tightly controlled to maintain the normal heart rhythm. Mutations in the SCN5A gene which encodes for the channel, can cause a broad range of cardiac pathologies and arrhythmias, including long QT syndrome (LQT3), Brugada syndrome (BrS), cardiac conduction defects, atrial fibrillation, and dilated cardiomyopathy. These can be severe enough to cause sudden infant death syndrome (SIDS), or sudden cardiac death in adults. The prevalence of congenital LQT syndrome is estimated at ˜1:2,500. Of these cases 5-10% are attributed to mutations in SCN5A (LQT3). However, the likelihood of dying during a cardiac event is 20% in families with an LQT3 mutation and 4% with either an LQT1 or an LQT2 mutation. Dysrhythmic symptoms can remain subclinical for prolonged periods and the triggers that activate an arrhythmia can be elusive. This highlights the need to study regulators of Nav1.5 to better understand how the channel can produce proarrhythmic behavior to identify therapeutic opportunities for these patients.

The phospholipid PIP₂ is a minor constituent of eukaryotic plasma membranes that is required for the physiological activity of most types of ion channels. In addition to its role in gating, PIP₂ also acts as an allosteric nexus that can regulate channel function by mediating the effects of drugs, post translational modifications, or protein partners. Despite the fundamental importance of PIP₂ to excitable physiology, the relationship between PIP₂ and Nav channels was only recently demonstrated when we showed that depleting PIP₂ enhances the function of Nav1.4, the isoform expressed in skeletal muscle. Dephosphorylating PIP₂ augments the overall activity of Nav1.4 and augments I_{LATE}. It is not known if PIP₂ regulates Nav1.5 or if interactions between PIP₂ and Nav1.5 are consequential in cardiomyocytes.

PIP₂ is rapidly hydrolyzed following activation of Gαq-coupled GPCRs. Cardiomyocytes express multiple types of Gαq-coupled receptors. Of these, the angiotensin II (Ang II) receptor AT1 and the endothelin (ET) receptor ET_{A} have direct clinical relevance in cardiovascular disease. Activation of AT1 by Ang II increases vascular resistance and cardiac output. Purified Ang II is used as a vasopressor in patients suffering from hypovolemic shock. In normotensive adults, the mean level of angiotensin II (Ang II) in arterial blood is 23 nM and this can double in essential hypertension and rise by as much as ten-fold in kidney disease. A prolonged elevation of Ang II levels, which occurs in heart failure, is associated with an increased risk of atrial fibrillation and ventricular arrhythmia. The commonly prescribed ACE inhibitors and angiotensin receptor blockers act by opposing Ang II and are associated with a reduced risk of ventricular arrhythmias.

ET is also a potent vasopressor with direct positive chronotropic and inotropic effects on the myocardium. ET levels can rise more than 5-fold within hours of a myocardial infarction, or during ischemia, increasing the risk of ventricular arrhythmia.

Discovered here is that PIP₂ is a regulator of Nav1.5 channel activity. A potential mechanistic link between Ang II, a cardiac signaling molecule associated with arrhythmia, and acute depletion of PIP₂ in Nav1.5 channels is tested. Furthermore, specific mutations associated with LQT3 reduce interactions between Nav1.5 and PIP₂, leading to cardiac disease and frequently death for these patients. This contributes to the phenotype of the mutation and renders these channels more sensitive to regulation by PIP₂ depletion. Together, these findings provide a holistic and mechanistic understanding of how PIP₂ modulates Nav1.5 activity and shapes the excitability of cardiomyocytes.

Showing that PIP₂ is a regulator of Nav1.5 channel activity provides new insights into the regulation of cardiac Nav channels in health and disease. Our data in human iPS cardiomyocytes suggest that Nav1.5 gating is modulated by Gαq-induced hydrolysis of PIP₂, including augmenting I_{LATE}. This proarrhythmic phenotype may be initiated by angiotensin II signaling. Furthermore, our data on the LQT3 mutation Nav1.5-R1644C support the finding that at least some disease mutations in Nav1.5 act to reduce the affinity of the channel for PIP₂. Consequently, the proarrhythmic features of these mutants are more likely to be evoked by Gαq-induced hydrolysis of PIP₂, or PIP₂ dephosphorylation.

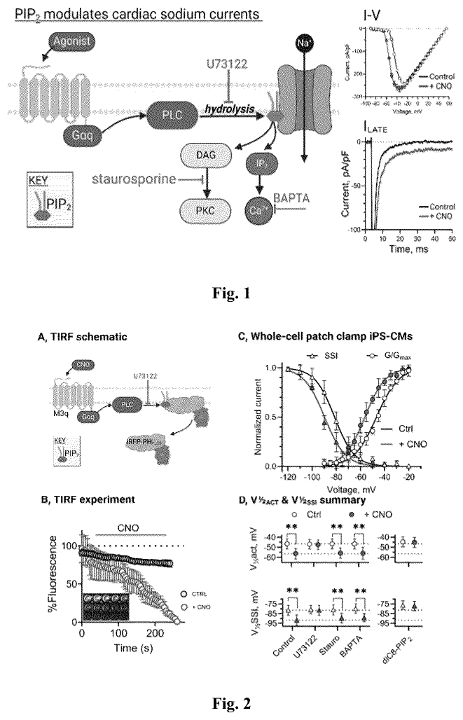

The experimental approach uses several innovative tools to study Nav1.5-PIP₂ interactions. First, we employ optogenetic (CRY2 system) and chemogenic tools (Designer Receptors Exclusively Activated by Designer Drugs or DREADD receptors) in concert with patch-clamp recording from human iPS-cardiomyocytes. The M3q DREADD receptor is a well-established tool that we use to initiate Gαq-signaling to hydrolyze PIP₂ in response to a specific ligand (clozapine N-oxide). This robust system allowed us to verify pharmacological tools that dissect the Gαq-signaling pathway to pinpoint hydrolysis of PIP₂ as the relevant effector, rather than downstream signaling from the resultant diacylglycerol (DAG) or inositol trisphosphate (IP3) (FIG. 1). PIP₂ hydrolysis occurs within tens of seconds of M3q activation, as shown by our total internal reflection fluorescence microscopy (TIRFM) studies using the iRFP-tagged PIP₂ biosensor PH-PLCS, which disengages from the membrane when PIP₂ is depleted. These studies are all paired with patch-clamp protocols to study the activation and inactivation gating of the cardiac sodium current, as well as the simultaneous, real-time change in I_{LATE} (FIG. 2). These DREADD receptor studies created a roadmap for to study the effects of PIP₂ hydrolysis following activation of the endogenous Gαq-coupled AT1 receptor, which is activated by angiotensin II. In contrast to PIP₂ hydrolysis, dephosphorylation of PIP₂ from PI-phosphatases does not generate downstream signaling molecules. Here, we employ specific, optogenetically activated PI-phosphatases, including a new tool, CRY2-pseudojanin, that we show is also efficacious in iPS-CMs. This robust optogenetic system uses blue light (˜440-490 nm) to photoactivate Cryptochrome 2 (CRY2) and its protein partner, CIBN. By fusing CIBN to a CAAX-domain, CRY2 and the phosphatase(s) fused to it are targeted to the inner leaflet of the cell membrane. Our show that photostimulation dephosphorylates PIP₂ in tens of seconds. Although PIP₂ levels can recover within minutes, its regeneration is suppressed by continued photostimulation. Our studies employ two distinct CRY2-fused PI-phosphatases. In addition to functional studies, we have developed a microscale thermophoresis (MST) approach to measure the binding affinity (Kd) of the soluble PIP₂ analog, diC8-PIP₂ for ion channels. This approach will be broadly applicable in the field but is used here to measure the Kd of diC8-PIP₂ for Nav1.5. We find that the Kd is ˜10-fold lower in the LQT3 mutant, Nav1.5-R1644C. Using molecular modeling, we predict that this R1644 forms a salt bridge with PIP₂.

This study uses complementary approaches to study interactions between PIP₂ and the cardiac Nav channel. Beyond the physiological relevance of the main target that PIP₂ regulates Nav1.5 channel activity, the experiments extend to cardiac disease. The role of PIP₂ in the activity of specific channelopathies and the regulation of Nav1.5 by PIP₂ hydrolysis mediated by Ang II signaling is evaluated.

Disclosed are methods of treating or preventing a cardiovascular disease, a pain syndrome, or epilepsy. The method may comprise administering to a subject in need thereof an effective amount of a compound. In some embodiments, the compound increases the interaction between NaV1.5 voltage-gated sodium channel or a NaV1.4 voltage-gated sodium channel and Phosphatidylinositol 4,5-bisphosphate (PIP2). In some embodiments, the compound is PIP2 or an analogue thereof. In some embodiments, the compound increases endogenous PIP2.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

For the purposes of this invention the term “therapeutically effective amount” of a therapeutic refers to the amount of the therapeutic which, when administered to a subject, elicits adequate therapeutic response in the subject to provide beneficial therapeutic outcome in the subject. A therapeutically effective amount may vary depending upon the intended application; the subject being treated, e.g., the weight and age of the subject; disease condition being treated, e.g., the severity of the disease condition; and the manner of administration. Based on these factors, a skilled artisan can determine a therapeutically effective amount of a therapeutic in a given situation.

As used herein, the term “administering” means providing a therapeutic agent or composition to a subject, and includes, but is not limited to, administering by a medical professional and self-administering. The means of providing a therapeutic agent are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

In certain embodiments, a therapeutic agent may be used alone or conjointly administered with another therapeutic agent. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

In certain embodiments, conjoint administration of the combinations of compounds of the invention with one or more additional therapeutic agent(s) (e.g., one or more additional chemotherapeutic agent(s)) provides improved efficacy relative to each individual administration of the combinations of compounds of the invention or the one or more additional therapeutic agent(s). In certain such embodiments, the conjoint administration provides an additive effect, wherein an additive effect refers to the sum of each of the effects of individual administration of the combinations of compounds of the invention and the one or more additional therapeutic agent(s).

The term “a small molecule” is a compound having a molecular weight of less than 2000 Daltons, preferably less than 1000 Daltons. Typically, a small molecule therapeutic is an organic compound that may help regulate a biological process.

“Subject” refers to an animal, such as a mammal, for example a human. The methods described herein can be useful in both humans and non-human animals. In some embodiments, the subject is a mammal (such as an animal model of disease), and in some embodiments, the subject is human.

The term “activator” refers to a compound having the ability to inhibit a biological function of a target biomolecule, for example, an mRNA or a protein, whether by increasing the activity or expression of the target biomolecule. Accordingly, the term “activator” is defined in the context of the biological role of the target biomolecule.

### PI(4,5)P₂ Regulates the Gating of Nav1.4 Channels

Nav channel gating is highly regulated to generate and support action potential firing in excitable tissues. In this study, optogenetic-activation of phosphoinositide-phosphatases reveals that PI(4,5)P₂ levels at the membrane tune the physiological gating behavior of Nav1.4 channels.

Voltage-gated sodium (Nav) channels are densely expressed in most excitable cells and activate in response to depolarization, causing a rapid influx of Na⁺ ions that initiates the action potential. The voltage-dependent activation of Nav channels is followed almost instantaneously by fast inactivation, setting the refractory period of excitable tissues. The gating cycle of Nav channels is subject to tight regulation, with perturbations leading to a range of pathophysiological states. The gating properties of most ion channels are regulated by the membrane phospholipid, phosphatidylinositol (4,5) bisphosphate (PI(4,5)P₂). However, it is not known whether PI(4,5)P₂ modulates the activity of Nav channels. Here, we utilize optogenetics to activate specific, membrane associated phosphoinositide (PI)-phosphatases that dephosphorylate PI(4,5)P₂ while simultaneously recording Nav1.4 channel currents. We show that dephosphorylating PI(4,5)P₂ left-shifts the voltage-dependent gating of Nav1.4 to more hyperpolarized membrane potentials, augments the late current that persists after fast inactivation, and speeds the rate at which channels recover from fast inactivation. These effects are opposed by exogenous diC8-PI(4,5)P₂. We provide evidence that PI(4,5)P₂ is a negative regulator that tunes the gating behavior of Nav1.4 channels.

Nav channels initiate and propagate action potentials in excitable cells. Their characteristic gating cycle, including voltage-dependent activation followed instantaneously by fast inactivation, serves as a ‘timer’ to reset the membrane potential, ensuring the generation of recurrent action potentials. Humans express nine Nav channels isoforms (Nav1.1-1.9) that are distinguished by differences in their gating kinetics, tissue distribution, and affinity for the pore-blocker tetrodotoxin (TTX). Each Nav channel is composed of a pore-forming a subunit that associates with one or two β subunits. The a subunit has four homologous domains (DI-DIV), each comprising six transmembrane α-helical segments (S1-S6) that incorporate an asymmetric loop located between S5 and S6, which lines the inner channel pore and forms the activation gate. S1-S4 form the voltage-sensing domain (VSD) with S4 carrying voltage sensing residues, typically positively charged arginines in every third position that move in response to depolarization to gate the channel. Domains I-III predominantly participate in the process of activation while DIII and DIV, along with the DIII-DIV linker, are crucial to the mechanism of fast inactivation.

All aspects of Nav channel gating are subject to stringent regulation to maintain the function of excitable tissues. Nav channelopathies caused by mutations, aberrant cell signaling pathways, or the off-target actions of drugs typically change the magnitude of Na⁺ currents. However, the underlying gating dynamics can be complex, in part because mechanical coupling between the domains of the Nav channel means that dysregulation in fast inactivation also shapes activation. Changes in gating behavior often lead to an increase in the magnitude of the current that persists after fast inactivation (called I_{LATE}), which contributes to the plateau phase of the cardiac action potential, resurgent excitability in neurons, and the excitability of skeletal muscle fibers. Therefore, even a small increase in I_{LATE} can precipitate diseases including cardiac arrhythmias, pain syndromes, epilepsy, and disorders of skeletal muscle contractility.

The phospholipid PI(4,5)P₂ is a minor constituent of eukaryotic plasma membranes that is required for the physiological activity of most types of ion channels. PI(4,5)P₂ is composed of a polar myo-inositol headgroup, coupled via a glycerol-phosphodiester linkage to arachidonic and stearic acid chains that permeate the inner leaflet of the membrane. The headgroup carries negatively charged phosphates at the 4 and 5 positions that interact with basic residues on partner proteins with a range of functional consequences. PI(4,5)P₂ is necessary in the gating process of many channel types, but also acts as an allosteric nexus that can regulate channel function by mediating the effects of drugs, post translational modifications, or protein partners. PI(4,5)P₂ levels are regulated by the activities of specific kinases and PI-phosphatases, which contribute to a dynamic equilibrium that modulates cell signaling and excitable behavior.

Although PI(4,5)P₂ has emerged as a master regulator of ion channel function, its role in the operation of Nav channel activity is unknown. Here, using optogenetic tools to activate specific membrane PI-phosphatases, we show that PI(4,5)P₂ plays a role in the physiological gating behavior of Nav1.4 channels. Dephosphorylating PI(4,5)P₂ left shifts the voltage-dependence of channel activation, speeds recovery from fast inactivation, slows the rate of fast inactivation, and augments I_{LATE}. Our data support the conclusion that PI(4,5)P₂ levels tune the activity of Nav1.4 channels.

We show that PI(4,5)P₂ is integral to multiple aspects of Nav1.4 channel gating, including regulating the voltage-dependence of activation, the rate of fast inactivation, the rate of recovery from fast inactivation, and the magnitude of the persistent current, I_{LATE}. To the best of our knowledge, this study is the first to reveal a role for PI(4,5)P₂ in the function of Nav channels. We find that depleting PI(4,5)P₂ augments the overall activity of Nav1.4, while enriching PI(4,5)P₂ levels right shifts the voltage-dependence of activation. Together, these findings show that PI(4,5)P₂ is negative modulator of Nav1.4, and suggest that PI(4,5)P₂ levels at the membrane tune the activity of the channel.

PI(4,5)P₂ is an established, inextricable cofactor in the gating of many types of ion channels, adopting a broad range of regulatory roles that vary with the structure and function of the channel under investigation. The role of PI(4,5)P₂ has been most extensively explored for inward rectifying potassium (Kir) channels. However, within the ion channel superfamily, voltage gated calcium (Cav) channels are the most structurally homologous to Nav channels. Extensive functional studies show that Cav channels exhibit dual regulation by PI(4,5)P₂. Thus, PI(4,5)P₂ stabilizes Cav channel currents by reducing ‘rundown’ while dephosphorylation of PI(4,5)P₂ results in a leftward-shift in the voltage-dependence of channel activation and inactivation, in an isoform dependent manner. Recently, structural analysis demonstrated that PI(4,5)P₂ associates with DII of Cav2.2, stabilizing the VSD in a ‘down’-conformation. PI(4,5)P₂ also impedes the rundown of voltage-gated potassium (Kv) channels and left-shifts the G-V relationship of Kv1 and Kv7 channels via interaction between PI(4,5)P₂ and the S4-S5 linker in the VSD of each protomer. Similarly, we find that dephosphorylation of PI(4,5)P₂ left-shifts the voltage-dependent gating of Nav1.4 channels.

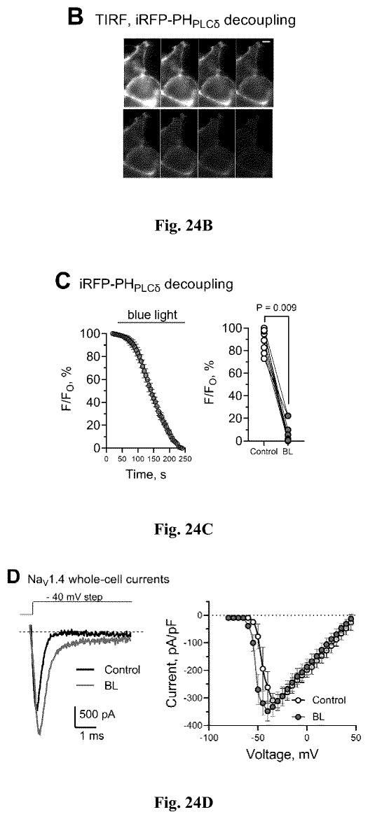

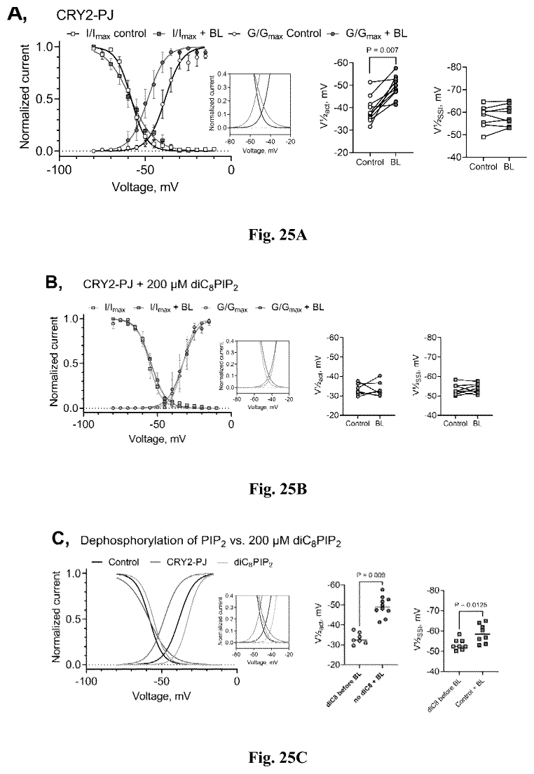

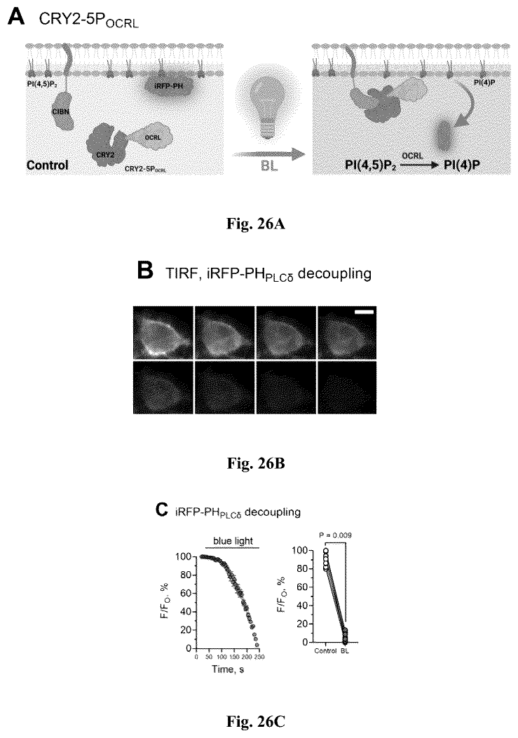

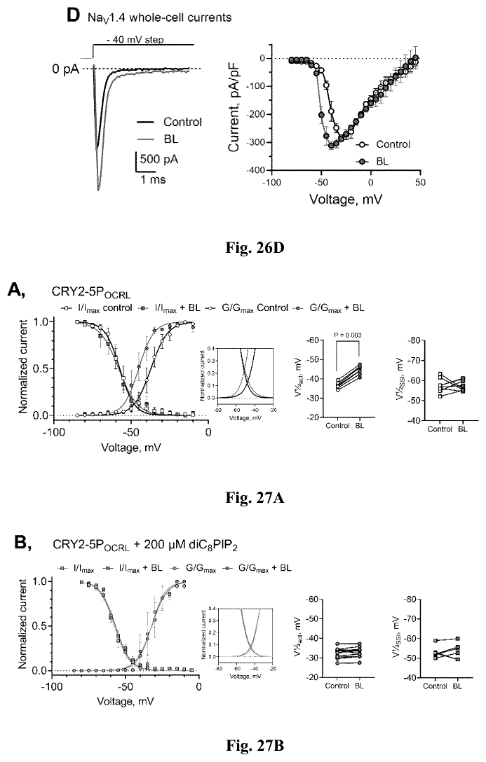

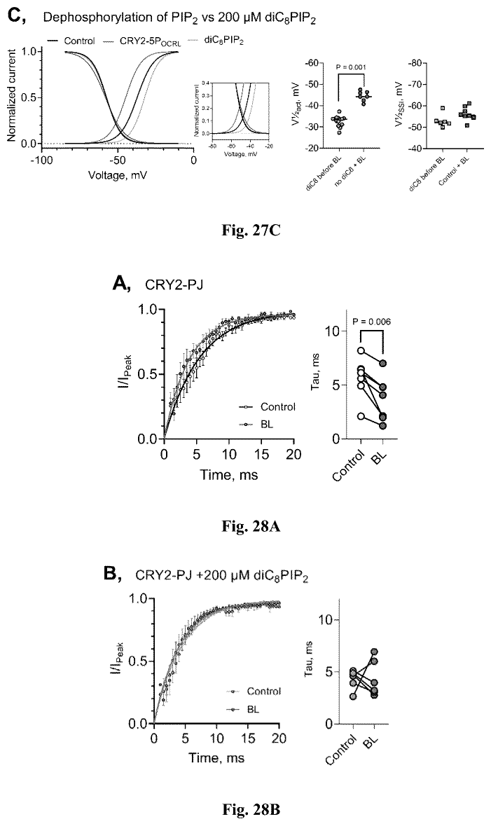

Although Nav channels are not typically associated with rundown, this phenomenon has been reported to occur for some Nav isoforms during longer recordings protocols, or when channels are studied in off cell patches. Current rundown has been attributed to the mechanosensitivity of Nav channels or the accumulation of channels in an inactivated state. However, in other ion channel families, rundown indicates a progressive loss of PI(4,5)P₂ from the local membrane environment. While we do not observe overt rundown of Nav1.4 channels, it is notable that the V½_{act} is right shifted when a high concentration of diC8PI(4,5)P₂ is included in the recording pipette (FIGS. 25 and 27). In general terms, the extent of channel rundown will depend on the affinity of the channel for PI(4,5)P₂, the abundance of PI(4,5)P₂ in the experimental system, and the ability of other PI-species to act as surrogate. This final parameter has been extensively studied for Kir channels with some members of the family, such as Kir2.1 showing exquisite selectivity for PI(4,5)P₂ while gating of other Kir channels, such as Kir6.2 can be supported by multiple PI-species (Rohacs et al., 2003). Our TIRF data show that CRY2-PJ and CRY2-5P_{OCRL} are equally effective at decoupling iRPF-PH_{PLCδ} from the cell membrane (FIGS. 24 and 26) but that the leftward shift in V½_{act} is ˜25% (−2.5 mV) less for CRY2-5P_{OCRL} than CRY2-PJ. Given that CRY2-5P_{OCRL} generates PI(4)P and CRY2-PJ generates PI, this apparent difference in V½_{act} might reflect that some aspects channel gating can be supported, at least in part, by PI(4)P.

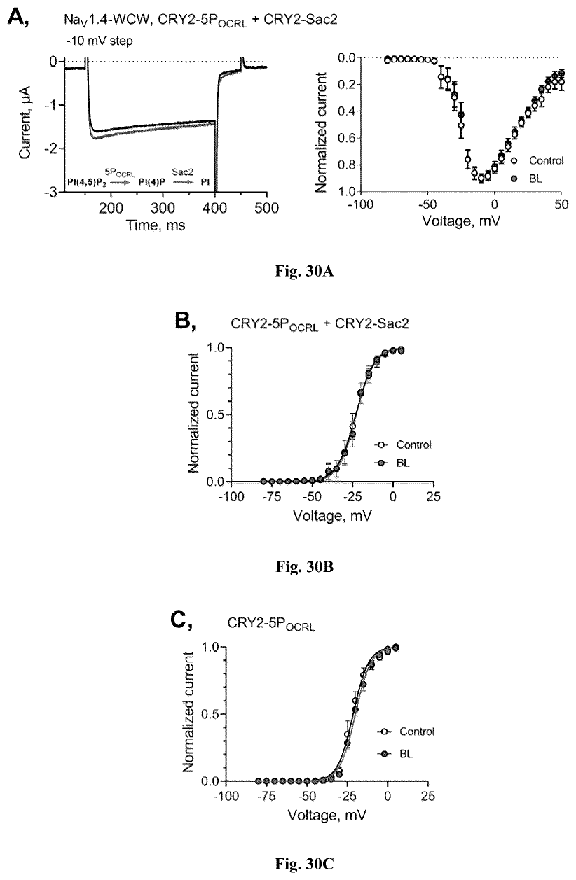

In skeletal muscle cells, a left shift in the voltage-dependence of Nav channel gating is expected to reduce the threshold for an action potential and change firing patterns. Given mechanical coupling between the domains of Nav channels, this excursion in the voltage-dependence of activation could also reflect changes in fast inactivation. Indeed, our studies with Nav1.4-WCW show that depleting PI(4,5)P₂ has limited impact on the G-V relationship of the channel in the absence of fast inactivation (FIG. 30). This finding suggests that PI(4,5)P₂ might play a role in the coupling between these processes. Similarly, the increased window current that results from PI(4,5)P₂ dephosphorylation suggests that there is a shift in equilibrium from inactivated to the open state of the channel which is corroborated by the increase in I_{LATE}. At the cellular level, the speeded rate of recovery from fast inactivation adds to this paradigm, enabling excitable cells to fire at a higher frequency, effectively shortening the refractory period. These data coalesce to predict a pattern of altered excitability in a PI(4,5)P₂ deficient environment, placing Nav1.4 channels under the direct oversight of PI(4,5)P₂ levels in the membrane. Although multiple disease-related mutations evoke similar dysregulation of Nav channel gating, whether dephosphorylation or hydrolysis of PI(4,5)P₂ plays a role in Nav channelopathies is yet to be determined.

Together, the data presented in this study show that PI(4,5)P₂ acts as a negative regulator of Nav1.4 function. Thus, dephosphorylation of PI(4,5)P₂ is expected to manifest detrimental changes in excitability. Our findings support the need for a structural understanding of Nav1.4-PI(4,5)P₂ association to delineate specific sites of interaction and to further determine the role of PI(4,5)P₂ in Nav channelopathies.

### MST Assay (Screening Assay)

The method utilizes cell lysates obtained from transfected/infected cells that are lysed and incubated with varying concentrations of a water-soluble analogue of PIP2 commercially available as diC8-PI(4,5)P₂, in a 384 well plate. Other phosphoinositides such as diC8PI(4)P of diC8PI(5)P can also be utilized. After a brief incubation (10-30 mins) samples are loaded onto capillary chips purchased from the company Nanotemper. Samples are run on the Nanotemper Monolith instrument. This instrument is advertised to detect green fluorescent protein (GFP) tagged proteins, however we have also used TFP (teal FP) tagged proteins. The instrument provides a short infra-red laser pulse which produces a short thermal gradient. Protein that is interacting with the lipid migrates differently through this thermal gradient and its movement is evaluated by the fluorescence of the GFP/TFP tag on the protein. Based on the concentration of the dic8-phosphoinositide, this resolves into a sigmoidal relationship that delivers an apparent affinity (Kd) value. A test ligand can be added to this assay at a known concentration that is at or close to its Kd for the channel. A shift in the channel's affinity for PIP2 in this assay can help indicate whether this test compound is a good drug candidate for further biological experiments.

In some embodiments, the purity of the lysate is increased using a pull down of the channel protein via the fluorescent tag, using a commercial nanobody designed against the GFP (or other fluorescent protein such as TFP). Drug screens can be performed against channel-PIP2 complex, for this, we incubate with the PIP2 at a high concentration and the drug at a range of concentrations, to look for strengthening or weakening of the PIP2 interaction. Secondarily, we can use a fluorophore on the PIP2 (commercially available BODIPY-tagged PIP2 from echelon, and perform the same experiment).

This invention will, for first time, provide an MST assay that is able to deliver an apparent Kd based on binding affinity rather than channel function which is a proxy for direct affinity estimates.
1. Purified protein is not needed for this MST assay unlike in other existing binding affinity assays. Since making purified protein is not always possible, this is a significant improvement over methods like surface plasmon resonance (SPR)
2. Cell lysates can be prepared in different cells lines like commonly used HEK293 cells and specialized cell lines typically used in protein production or specialized equipment are not required.
3. Both GFP and TFP tagged proteins can be used for this assay. This instrument is marketed toward use in the estimates of GFP tagged protein affinity for nucleic acids or other proteins. Our assay can also handle TFP tagged proteins that uses slightly different fluorescence excitation and emission frequencies.
4. Due to the minute scale of the assay, small quantities of lipid are required, unlike SPR, even in high-throughput screening formats.

Mutations in ion channels often cause diseases like cardiac arrhythmia, epilepsy and muscular dystrophy and a quantification of mutant-PIP2 affinity can establish whether the change in channel function is due to the deterioration of the channel-PIP2 relationship in the mutant. This invention will, for first time, provide drug discovery strategies that can work through remedying this alteration in the mutant-PIP2 relationship and reverse the change in channel function in disease.

This technology can be used in drug screening targeting various ion channels and has been tested in different ion channel families. This assay uses raw material (cell lysate) that is obtained through processes commonly available in most biology labs. No specialized equipment or reagents are required to prepare material that needs to be tested. Additionally, the high cost in terms of reagents and man-hours required to produce purified protein used in an SPR assay that is the only other assay that can be used to generate data of this type is subverted, making this MST assay easier to adopt on a larger scale. This assay can be performed for high throughput screening of drug candidates to correct ion channelopathies. Data is analyzed by the software package pre-loaded on the instrument.

The present invention provides methods of identifying a compound that modulates the interaction between a NaV1.5 voltage-gated sodium channel or a NaV1.4 voltage-gated sodium channel and PIP2. The method may comprise contacting cells expressing a tagged NaV1.5 channel (or a variant thereof), or a tagged NaV1.4 channel (or a variant thereof) with a candidate agent and PIP2 or a PIP2 analog (such as diC8PI(4,5)P2). In other embodiments, the method comprises contacting cell lysate of cells expressing a tagged NaV1.5 channel (or a variant thereof), or a tagged NaV1.4 channel (or a variant thereof) with a candidate agent and PIP2 or a PIP2 analog (such as diC8PI(4,5)P2). The method may further comprise detecting the affinity of PIP2 or the PIP2 analog to the tagged NaV1.5 channel or the tagged NaV1.4 channel. The method may further comprise comparing the affinity in the presence of the candidate agent with the affinity in the absence of the candidate agent; wherein a change in affinity in the presence of the candidate agent is indicative of modulation of the interaction between NaV1.5 or NaV1.4 and PIP2.

In some embodiments, the tagged NaV1.5 channel, the tagged NaV1.4 channel, or a variant thereof is isolated from the cell lysate before incubating with the candidate agent and PIP2 or the PIP2 analog. In some embodiments, the tagged NaV1.5 channel, the tagged NaV1.4 channel, or a variant thereof is tagged with a fluorescent protein. In some embodiments, the fluorescent protein is a green fluorescent protein (GFP), a yellow fluorescent protein (YFP), a red fluorescent protein (RFP), or a monomeric teal fluorescent protein (mTFP). In some embodiments, the affinity is detected via an instrument detecting the tagged protein. In some embodiments, the candidate agent is a small molecule or a peptide.

### PI(4,5)P₂ is a Negative Modulator of Cardiac Sodium Channels

The cardiac voltage-gated sodium (Na⁺) channel 1.5 (Naᵥ1.5) is the predominant voltage-gated sodium channel in the heart. Naᵥ1.5 channels open upon depolarization and transport Na⁺ along the electrochemical gradient. The initial upstroke of the cardiac action potential is driven by this inflow of Na⁺. Rapid activation and inactivation of Naᵥ1.5 channels are crucial for the initiation and propagation of action potentials (APs) in cardiac myocytes. Dysfunction in Naᵥ1.5 channel activity has been associated with numerous congenital and acquired cardiac diseases. A common factor among these arrhythmic syndromes is the dangerous elevation in persistent or late sodium current (I_{LATE}). Here in both rat ventricular cardiomyocytes (RVCMs) and HEK cells, we show for the first time using optogenetics that phosphatidylinositol (4,5) bisphosphate (PI(4,5)P₂) underlies Naᵥ1.5 channel gating. We show that the simultaneous use of both whole-cell patch-clamp and optogenetics to target phosphatase to the membrane to dephosphorylate PI(4,5)P₂, left-shifts the voltage dependence of both activation and inactivation and increases I_{LATE}. These effects are challenged by the application of exogenous diC8-PI(4,5)P2. This evidence is consistent with PI(4,5)P₂'s ability to regulate the gating behavior of numerous other ion channels including Naᵥ1.4.

The cardiac voltage-gated sodium channel 1.5 (Nav1.5) encoded by the SCN5A gene is a member of the voltage-gated ion channel superfamily. It is the predominant sodium channel isoform in cardiomyocytes. Here, it is important for the initiation and propagation of the cardiac action potential. Dysfunction of the Naᵥ1.5 channel due to various mutations in the SCN5A gene has been associated with many cardiac diseases thereby, highlighting the therapeutic need. A unique commonality among all these arrhythmic syndromes is the availability of a persistent Na⁺ current (I_{LATE}) that manifests due to disruption in the channel gating.

Naᵥ1.5 channels consist of a pore-forming α-subunit which is composed of four domains named domain I (DI) to domain IV (DIV) that are linked by cytoplasmic loops. Each of these domains consists of six transmembrane segments named S1-S6. The pore domain is composed of transmembrane S5 and S6 which are important for both the selectivity and permeation of ions. Domains I-III participates in the rapid activation of the channel which produces sodium current (I_{Na}). Here, the transmembrane segments S1-S4 form the voltage-sensing domain. Every third residue in the S4 segment is typically a positive arginine which shifts in response to depolarization to activate the channel. Shortly thereafter, Domains III, DIV, and the DIII-DIV linker work in concert to quickly inactivate the channel.

The transition from open to inactivated conformations is a very fast process, starting immediately after depolarization and rendering almost all channel complexes blocked for conductance after a few milliseconds. However, a small fraction of channels does not inactivate completely and instead remain open, producing I_{LATE}. Under physiological conditions, the I_{LATE} plays a role in sustaining the action potential plateau. In cardiac diseases such as long-QT syndrome type 3 (LQT3) and Brugada syndrome (BrS), there is a significant increase in I_{LATE} and a shift in the voltage dependence of the channel. Increased I_{LATE} shifts the equilibrium towards proarrhythmic conditions which results in the prolongation of the action potential duration (APD), thus, predisposing individuals to lethal cardiac conditions. These observations therefore highlight the therapeutic potential of targeting I_{LATE}.

Phosphatidylinositol-4,5-bisphosphate, or PI(4,5)P₂ is a phospholipid which produces both inositol-1,4,5-triphosphate (IP3) and 1,2-diacylglycerol (DAG). These secondary messengers (IP3 and DAG) that are generated because of PI(4,5)P₂ hydrolysis by phospholipase C (PLC) are important in many signaling pathways. Phospholipid PI(4,5)P₂, is composed of a polar myo-inositol headgroup which contains negatively charged phosphates at positions 4 and 5 of the inositol ring. The phospholipid also consists of two fatty acid tails. PI(4,5)P₂ is an important regulator of many ion channels and transporters. Many ion channels including inward-rectifying K+ (Kir), voltage-gated K⁺ (Kv), two-pore domain K⁺ (K2P), voltage-gated calcium (Ca²⁺), transient receptor potential canonical (TRPC), and epithelial Na⁺ channels require PI(4,5)P₂ to maintain channel activity and gating.

We have shown using an optogenetic system that allows us to spatially and temporally manipulate the availability of PI(4,5)P₂ at the cellular membrane that the voltage-gated sodium channel Nav1.4, the skeletal sodium channel, is regulated by PI(4,5)P₂. Dephosphorylation of PI(4,5)P₂ evoked left shifts in both the voltage-dependence of activation and inactivation of Naᵥ1.4 and increase in I_{LATE}. Given the sequence similarities amongst voltage-gated sodium channels and the importance of I_{LATE} in cardiac disease, we tested that Naᵥ1.5 channels would similarly be regulated by PI(4,5)P₂.

In this study, we show that use of optogenetic tools to target specific phosphatase for PI(4,5)P₂ alters the gating behavior of Naᵥ1.5 channels. As with Naᵥ1.4, dephosphorylation of PI(4,5)P₂ left shifts the voltage-dependence of activation of Nav1.5. However, unlike the Naᵥ1.4 channel we report that dephosphorylation of PI(4,5)P₂ noticeably left shifts the voltage-dependence of inactivation. In both Naᵥ1.4 and Naᵥ1.5 dephosphorylation of PI(4,5)P₂ augments I_{LATE}. Our findings show that Naᵥ1.5 is negatively regulated by PI(4,5)P₂ and its regulation of Naᵥ1.5 channels is important for normal cardiac physiology and pathology.

### PI(4,5)P₂ Modules Nav1.5 Channel Activity

Nav1.5 and Kir2.1 channels are regulated by SUMOylation, a post translational modification pathway in cardiomyocytes. The pro-arrhythmic effects of SUMO occur due to a complementary mechanism: SUMOylation augments the activity of Nav1.5 and decreases the activity of the inward rectifying K⁺ current I_{K1}, that is primarily carried by Kir2.1. Combining optogenetic tools and genetic manipulations, we showed that SUMO inhibits I_{K1} by reducing the efficacy of the phospholipid PI(4,5)P₂ to activate Kir2.1 gating. However, the effect of PI(4,5)P₂ on the function of Nav1.5 channels is unknown and is a gap in knowledge that we aim to address through this proposal. Our data show that depleting PI(4,5)P₂ from the membrane of cardiomyocytes and heterologous cells increases the activity of Nav1.5 in a proarrhythmic manner, allowing channels to open at less depolarized membrane voltages and augmenting I_{LATE}. These preliminary findings suggest that PI(4,5)P₂ is a negative modulator of Nav1.5 and dephosphorylation or depletion of PI(4,5)P₂ increases excitability.

PI(4,5)P₂ is a negative modulator of Nav1.5 channel activity.

Our work on SUMOylation and the regulation of ion channels by acute hypoxia led us to dissect the role of PI(4,5)P₂ in mediating the effects of SUMO on I_{K1}. Based on the parallels between our studies on SUMO-regulation of Kir2.1 and Nav1.5 channels, we performed preliminary studies to investigate the PI(4,5)P₂ dependence of Nav1.5 channels. This trajectory required significant method development to optimize the optogenetic-based PI-phosphatase probes that we use to dephosphorylate PI(4,5)P₂ and PI(4)P. Using Nav1.4 as a model system, we performed the first study to show PI(4,5)P₂ modulation of a voltage-gated Na⁺ channel. Our data reveal that Nav1.5 is also subject to PI(4,5)P₂ modulation, suggesting this mode of regulation is likely generalizable across the Nav channel family, with PI(4,5)P₂ acting as a negative modulator that decreases excitability. Further, using RVCMs we provide the first evidence that PI(4,5)P₂ modulates native Nav channel complexes. It is notable that at the cellular level, dephosphorylating PI(4,5)P₂ decreases the activity of many K⁺ channels and augments Nav currents, including in cardiomyocytes. The experiments described in this proposal will define if this thematic innovation extends further, as it does in K⁺ channels, with PI(4,5)P₂ acting as an allosteric hotspot that coordinates the action of channelopathies and PTMs, such as SUMOylation.

To study dephosphorylation of PI(4,5)P₂ and PI(4)P, we employ specific, optogenetically activated PI-phosphatases, including a new tool, CRY2-pseudojanin, that we show is also efficacious in RVCMs. This robust optogenetic system uses blue light (˜440-490 nm) photoactivation of Cryptochrome 2 (CRY2) and its protein partner, CIBN. By fusing CIBN to the membrane anchor CAAX, CRY2 and the phosphatase(s) fused to it are targeted to the inner leaflet of the cell membrane. Photostimulation dephosphorylates PI(4,5)P₂ in tens of seconds. Therefore, PI(4,5)P₂ biosensors like the pleckstrin-homology domain of phospholipase C (PLCS-PH) rapidly disengage from the membrane within tens of seconds of blue light illumination. Although PI(4,5)P₂ levels can recover within minutes, regeneration of PI(4,5)P₂ is suppressed by continued photostimulation. Our studies employ two distinct CRY2-fused PI-phosphatases. We dephosphorylate PI(4,5)P₂ at the 4 and 5 positions using pseudojanin (CRY2-PJ), a construct that encompasses an inositol polyphosphate 5-phosphatase that generates PI(4)P from PI(4,5)P₂, and the 4-phosphatase Sac that generates PI from PI(4)P). To test if PI(4)P modulates the gating of Nav1.5, we use CRY2-5-phosphatase_{OCRL} (CRY2-5ptase), the inositol polyphosphatase 5-phosphate found at the Lowe oculocerebrorenal syndrome gene locus (OCRL). We and others have previously used this construct to study the effects of 5-dephosphorylation of PI(4,5)P₂ on KCNQ, Kir2, Kir3, TRPC5 and ENaC channels.

Our approach uses complementary approaches to characterize the relationship between PI(4,5)P₂ and the function of the cardiac Nav channel, including testing channelopathies and endogenous regulators, such as SUMO.

### Methods

The activity of voltage-gated sodium channels is tuned by a specific lipid in the cell membrane called PIP2. Thus, depleting PIP2 levels can cause sodium channel dysregulation, initiating the aberrant behaviors observed in disease states. Conversely, disease mutant channels can be made to act like regular channels when we enrich PIP2 levels in the cell. Understanding the relationship between Nav and PIP2 is key to developing new treatments for Nav-related disease states.

This invention is based upon the new discovery that PIP2 regulates Nav channels, and that PIP2 may be enriched to reduce the effect of disease mutations. Described herein is the elucidation of the role of PIP2 in mediating the effects of disease mutations and phenotypes from mutant Nav channels. This disclosure outlines a mechanism that explains why the disease mutant Nav channels behave differently under specific circumstances (when PIP2 levels change).

The discoveries presented herein provides routes for treating Nav-related disease states that provide advantages over current treatments. For example, existing anti-arrhythmic drugs often have side effects, several existing pain relief medications can be addictive, and there are currently no useful drugs for paralytic disorders. The discoveries presented herein enable the design of drugs that modulate Nav1.x-PIP2 interactions to act as anti-arrhythmic, anti-PMC/periodic paralysis, anti-epileptic, or anti-pain (analgesic) medications. Based on the mechanism, these drugs are not expected to be addictive.

Disclosed are methods of treating or preventing a cardiovascular disease, a pain syndrome, epilepsy, or a skeletal muscle disease. The method may comprise administering to a subject in need thereof an effective amount of a compound. In some embodiments, the compound increases the interaction between NaV1.5 voltage-gated sodium channel or a NaV1.4 voltage-gated sodium channel and Phosphatidylinositol 4,5-bisphosphate (PIP2). In some embodiments, the compound is an analogue of PIP2, such as diC8-PIP2. In some embodiments, the compound is PIP2. In some embodiments, the compound increases endogenous PIP2.

In some embodiments, the compound increases endogenous PIP2; and the compound is an inhibitor a Gαq-coupled AT1 receptor, such as 3 losartan, Exp 3174, telmisartan, irbesartan, candesartan, valsartan, eprosartan, azilsartan, saprisartan or olmesartan. In some embodiments, the compound increases endogenous PIP2; and the compound is an inhibitor of phospholipase C (PLCO), such as U73122, phenylmethylsulfonyl fluoride, manoalide, D609, ET-18-OCH3, compound 48/80 trihydrochloride, spermine tetrahydrochloride, neomycin sulfate, NCDC, or thielavin B. In some embodiments, the compound increases endogenous PIP2; and the compound inhibits PIP2 hydrolysis or inhibits PIP2 dephosphorylation.

In some embodiments, a cardiovascular disease is treated or prevented; and the cardiovascular disease is arrhythmias, long QT syndrome (LQT3), Brugada syndrome (BrS), cardiac conduction defects, atrial fibrillation, and dilated cardiomyopathy, sudden infant death syndrome (SIDS), or sudden cardiac death in adults. In some embodiments, a pain syndrome is treated or prevented; and the pain syndrome is a chronic pain syndrome, fibromyalgia, or neuropathic pain. In some embodiments, epilepsy is treated or prevented; and the epilepsy is an idiopathic generalized epilepsy, idiopathic partial epilepsy, symptomatic generalized epilepsy or symptomatic partial epilepsy. In some embodiments, the symptomatic partial epilepsy is temporal lobe epilepsy. In some embodiments, the subject suffers from a traumatic brain injury, hypoxic brain injury, brain infection, stroke, or genetic syndrome. In some embodiments, the subject suffers from a brain infection; and the brain infection is encephalitis, meningitis, mesial temporal sclerosis, or a cerebral tumor. In some embodiments, the epilepsy is at least partially induced by the traumatic brain injury, hypoxic brain injury, brain infection, stroke, or genetic syndrome. In some embodiments, the epilepsy is a TBI-induced epilepsy. In some embodiments, a skeletal muscle disease is a periodic paralysis (PP), a nondystrophic myotonia (NDM), and a ryanodinopathy. In some embodiments, the periodic paralysis (PP) or the nondystrophic myotonia (NDM) is myotonia congenita, paramyotonia congenita (PMC), or potassium-aggravated myotonia (PAM), hyper and hypokalemic periodic paralysis (hyperPP and hypoPP), or Andersen-Tawil syndrome (ATS). In some embodiments, the ryanodinopathy is malignant hyperthermia (MH), central core disease (CCD), multi-minicore disease (MmD), or centronuclear myopathy (CNM).

### EXAMPLES

The invention now being generally described, it will be more readily understood by reference to the following examples, which are included merely for purposes of illustration of certain aspects and embodiments of the present invention, and are not intended to limit the invention.

### Example 1: PIP2 Modulates the Activity of Nav1.5 Channels

Regulation of Nav1.5 channels: Nav1.5 channels open when cardiomyocytes depolarize, generating I_{Na}, the rapidly activating inward Na⁺ current that underlies the upstroke of the cardiac action potential. Activation of Nav1.5 triggers fast inactivation, terminating I_{Na} and allowing the myocardium to repolarize. Nav1.5 channels also pass a persistent current (I_{LATE}) that is typically less than 0.5% of the magnitude of peak I_{Na}. Cardiovascular diseases such as myocardial ischemia, heart failure, or mutations associated with LQT3 syndrome can increase I_{LATE} up to 2-5% of peak I_{Na}. This abnormal elevation in I_{LATE} means that Na⁺ ions leak into cardiomyocytes, prolonging repolarization and reducing repolarization reserve, heightening the risk of irregular heart rhythms, sudden infant death syndrome (SIDS), and sudden cardiac death. Pathophysiological changes in cardiac tissue, such as hypoxemia and acidosis can also disrupt the gating cycle of Nav1.5 and cause a proarrhythmic increase in I_{LATE}.

Nav channels and PIP₂: The activity of Nav1.5 is controlled by various auxiliary protein partners and post-translational modifications that regulate the channel in response to hormones, neurotransmitters, and cell signaling pathways. The phospholipid phosphatidylinositol 4,5-bisphosphate (PIP₂) is a minor constituent of eukaryotic plasma membranes that is required for the physiological activity of most types of ion channels. PIP₂ is composed of a polar myo-inositol headgroup, coupled via a glycerol-phosphodiester linkage to arachidonic and stearic acid chains that permeate the inner leaflet of the membrane. The headgroup carries negatively charged phosphates at the 4 and 5 positions that interact with basic residues on partner proteins with a range of functional consequences. PIP₂ is necessary in the gating process of many channel types, but also acts as an allosteric nexus that can regulate channel function by mediating the effects of drugs, post translational modifications, or protein partners. A relationship between PIP₂ and Nav channels was demonstrated showing that depleting PIP₂ enhances the function of Nav1.4, the isoform expressed in skeletal muscle. Dephosphorylating PIP₂ augments the overall activity of Nav1.4 by left shifting the voltage-dependence of activation (V½_{ACT}), slowing the rate of fast inactivation, and augmenting I_{LATE}. In contrast, enriching PIP₂ levels right shifted the V½_{ACT}, suggesting that PIP₂ is a negative modulator of Nav1.4 channel activity. Our findings led to further investigating PIP₂ regulation of other Nav channel isoforms, including Nav1.5, the effect of PIP₂ on Nav1.5, PIP₂ regulation of Nav1.5 consequential in cardiomyocytes, and the relationship between Nav1.5 and PIP₂ impacted by cell signaling pathways that play a role in cardiovascular physiology and disease. PIP₂ dephosphorylation was accomplished by combining whole-cell patch-clamp recording with photoactivation of membrane targeted phosphoinositol (PI)-phosphatases that dephosphorylate PIP₂. This robust optogenetic platform is based on blue light (˜440-490 nm) photoactivation of Cryptochrome 2 (CRY2) and its protein partner, CIBN. Fusing CIBN to the membrane anchor CAAX targets CRY2 and its cargo to the inner leaflet of the cell membrane in response to photostimulation.

PIP₂ levels are also regulated by hydrolysis following activation of Gαq-coupled GPCRs. Activated Gαq proteins turn on phospholipase C (PLCβ), which hydrolyzes PIP₂ into inositol 1,4,5-trisphosphate (IP3) and diacylglycerol (DAG; FIG. 1). Over tens of minutes to hours, DAG decreases I_{Na} via PKC-mediated phosphorylation of Nav1.5, which right shifts the voltage-dependence of inactivation (V½_{SSI}) and promotes the recycling of the channels from the plasma membrane. These effects are blocked by kinase inhibitors like staurosporine. IP3 causes the release of Ca²⁺ ions from intracellular stores. The rise in intracellular Ca²⁺ has been associated with a rightward shift in V½_{SSI} and a reduction in I_{LATE}. These effects are ameliorated by Ca²⁺-chelators, like BAPTA. The acute effects of PIP₂ hydrolysis on the channels are unknown.

Gαq signaling in cardiomyocytes: Cardiomyocytes express multiple types of Gαq-coupled receptor, typically at low levels. Of note, the angiotensin II (Ang II) receptor AT1 and the endothelin (ET) receptor ET_{A} are more robustly expressed and have direct clinical relevance in cardiovascular disease. Ang II is produced by the renin-angiotensin-aldosterone system (RAAS) and acts at AT1 and AT2 G protein-coupled receptors (GPCRs) in cardiomyocytes and vascular smooth muscle to increase vascular resistance and cardiac output. Ang II is indicated for use as a vasopressor in patients suffering from hypovolemic shock, for example in sepsis. In normotensive adults the mean level of Ang II in arterial blood is 2.4±1.2 μg/100 ml blood (−23 nM). Ang II levels can double in essential hypertension and rise by ten-fold, exceeding 200 nM in renal hypertension. A prolonged elevation of Ang II levels, which occurs in heart failure, is associated with hypertension, cardiac hypertrophy, and fibrosis, and an increased risk of atrial fibrillation and ventricular arrhythmia. Indeed, many of the most prescribed anti-hypertensive drugs act by opposing Ang II (Angiotensin converting enzyme, ACE inhibitors, or angiotensin receptor blockers, ARBs), and are associated with a reduced risk of ventricular arrhythmias. ET is also a vasopressor, exerting positive chronotropic and inotropic effects on the myocardium and constricting coronary artery smooth muscle cells. ET levels can rise more than 5-fold within hours of a myocardial infarction, or cardiac ischemia increasing the risk of ventricular arrhythmia.

Results. Studies are designed to evaluate if hydrolysis of PIP₂ modulates Nav1.5 channel currents in cardiomyocytes. To perform these experiments, we used human iPS cardiomyocytes (iCell² iPS-CMs) from Fuji-Film Cellular Dynamics. These iPS-CMs are a valuable model system for human cardiomyocytes that retain many of the key characteristics of the native cells. Here, we transfected the iPS-CMs to express the Gαq-coupled M3q Designer Receptors Exclusively Activated by Designer Drugs (DREADD) receptor. We use a magnetic transfection system from Oz Biosciences that we have previously employed with cardiomyocytes. First, we tested the efficacy of M3q to hydrolyze PIP₂ in our model system. To accomplish this, we studied uncoupling of the PIP₂ biosensor iRFP-PH_{PLCδ1} from the plasma membrane in response to chemogenic activation of M3q by 100 nM clozapine N-oxide (CNO, Tocris Biosciences). Using total internal reflection fluorescence microscopy (TIRFM) we showed that iRFP-PH_{PLCδ1} uncouples rapidly from the membrane in response to M3q activation, and completely within 200s (FIGS. 2A, 2B).

These findings are consistent with our prior observations and the work using optogenetically activated PI-phosphatases to deplete PIP₂ in HEK293 cells of Xenopus laevis oocytes. Uncoupling does not occur if M3q is not expressed, or if the cells are treated with the PLCβ inhibitor, U73122 (5 μM, Tocris Biosciences). Having established a time frame for PIP₂ hydrolysis in our system, we used whole-cell patch-clamp to study I_{Na} in paired experiments, before and after chemogenic activation of M3q. Applying 100 nM CNO evoked a leftward shift in the current-voltage (I-V) relationship of I_{Na} such that peak I_{Na} shifted from −25 to −35 mV. At −35 mV, the peak current-density increased from −220±5 pA/pF to −266±4 pA/pF at −35 mV (n=8, P<0.001, paired Student's t-test). Fitting the data with a Boltzmann function to obtain a conductance-voltage (G-V) relationship revealed a −10±5 mV leftward shift in the mean half-maximal activation voltage of I_{Na} (V½_{ACT}) without a change in the slope factor (n=8, P<0.01, paired t-test). These results are similar to those obtained from Nav1.4 following optogenetic dephosphorylation of PIP₂. However, in contrast to our findings with Nav1.4, activating M3q signaling also left-shifted the steady state inactivation curve of Nav1.5 (V½_{SSI}), from −81.4±2 to −89.7±2 mV (n=8, P<0.01, paired t-test; FIGS. 2C, 2D). These studies indicate that Nav1.5 channels are regulated by hydrolysis of PIP₂ and that there are distinctions between PIP₂ regulation of Nav1.5 and Nav1.4.

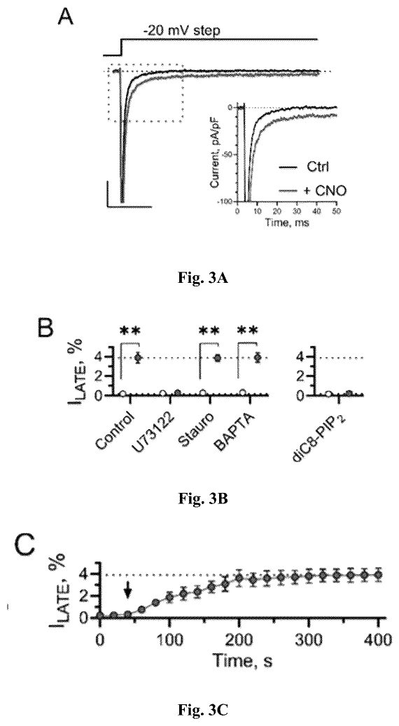

Next, we studied the effect of M3q-activation on I_{LATE} by recording currents evoked by a 400 ms depolarizing pulse to −20 mV before and during receptor activation. The magnitude of I_{LATE} represents the mean current 150-200 ms after peak I_{Na} and is expressed as a percentage of peak I_{Na}. Exposure to 100 nM CNO resulted in a 16-fold increase in I_{LATE} from control levels of 0.22% to ˜3.9% of peak I_{Na} (n=8, P<0.001; paired t-test). Importantly, the rate of I_{LATE} elevation aligned with the TIRFM data demonstrating PIP₂ hydrolysis. The effects of M3q activation on V½_{ACT}, V½_{SSI}, and the development of I_{LATE} were not affected by staurosporine (1 μM, 1-hour preincubation) or BAPTA (10 mM in the recording pipette), making a role for IP3 or DAG unlikely. In contrast, the effects of CNO were blocked when the cells were treated with the PLCβ inhibitor, U73122 (5 μM), or by the inclusion of the soluble PIP₂ analogue diC8-PIP₂ in the pipette (200 μM), indicating that PIP₂ hydrolysis is both necessary and sufficient (FIG. 3). Similar data were obtained for Nav1.5 expressed with Navβ1 and the M3q-DREADD receptor in HEK293T cells. This experiment validates that the findings are not restricted to a particular model system or cellular environment.

Experimental Design. Our data show that PIP₂ hydrolysis induced by chemogenic stimulation of heterologously expressed DREADD M3q receptors leads to significant leftward shifts in the voltage-dependent gating of I_{Na} (V½_{ACT} and V½_{SSI}), and augments I_{LATE}. Experimental sub-aims designed to study (1) if I_{Na} and I_{LATE} in iPS-CMs can be regulated by endogenous Gαq-coupled receptor-initiated hydrolysis of PIP₂ and (2), the role of PIP₂ in the function of Nav1.5 channels are proposed.

Measure modulation in I_{Na} and I_{LATE} by endogenous Gαq signaling. Like native cardiomyocytes, human iPS-CMs express AT1 receptors. Here, the effects of Ang II (Tocris) on Nav1.5 channel function in iPS-CMs is tested using four whole-cell patch-clamp recording protocols. Recording solutions and amplifier settings are described in the legend of FIG. 2:
i) I-V (and G-V): The current-voltage (I-V) relationship reports on current-density across the physiological voltage range and provides a conductance voltage (G-V) relationship that is normalized and fitted with a Boltzmann function to illustrate the voltage-dependence of activation, V½_{ACT}.
ii) SSI: a paired-pulse protocol to determine the voltage-dependence of steady-state inactivation (SSI). The prepulse is incrementally depolarized while the test-pulse remains at a fixed amplitude. Fitting this data with a Boltzmann function gives V½_{SSI}.
iii) RFI: cellular excitability is shaped by the rate at which Nav channels recover from fast inactivation (RFI) and become available to initiate an action potential. This parameter is studied using paired steps to −30 mV and incrementally increasing the duration between the pulses.
iv) I_{LATE} is studied using 400 ms depolarizing pulses to −20 mV. The pulse is repeated every 10 seconds for the duration of a time-course study. Subsequently, the cell is exposed to the state-dependent, late-current blocker ranolazine at 1 μM, as previously described. The magnitude of I_{LATE} represents the mean current 150-200 ms after peak I_{Na} and is expressed as a percentage of peak I_{Na}, following ranolazine subtraction. The time course of a change in I_{LATE} and the change in the time constant of fast inactivation are evaluated.

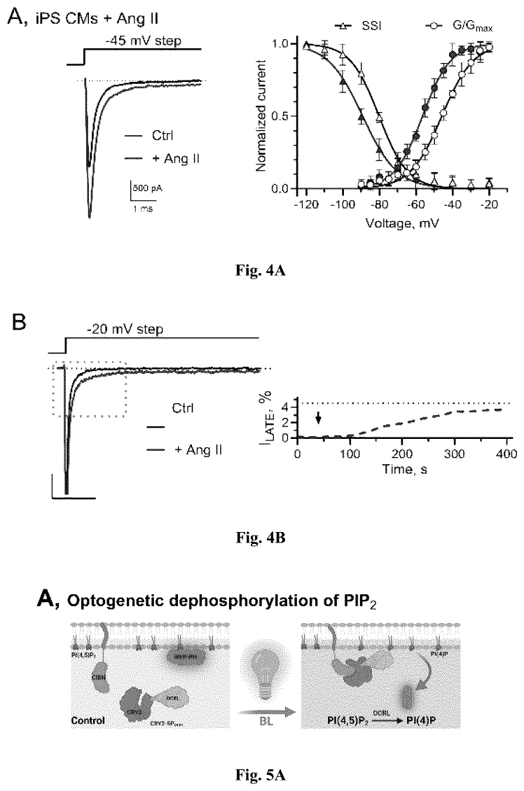

In each case, Nav1.5 currents is studied before and after the administration of Ang II. We tested 100 nM Ang II and observed an increased in peak I_{Na}, a leftward shift in V½_{ACT} and V½_{SSI} of −7 mV and an increase in I_{LATE} from 0.15±0.1 to 3.6±0.3% of I_{PEAK} over ˜250 seconds (n=4, P<0.01 t-test) (FIG. 4). Next, we study the concentration-dependence of these effects, spanning the pathophysiological range (20-200 nM). Utilizing a non-saturating concentration of Ang II, we introduce pharmacological tools to determine the effects on Nav1.5 function attributable to PIP₂ hydrolysis. Initially, we test the requirement for the AT1 receptor using the AT1-selective blocker losartan at 200 nM (IC₅₀, 20 nM, Tocris Biosciences). Subsequently, we block PIP₂ hydrolysis using the PLCβ inhibitor, U73122 (5 μM, Tocris Biosciences). As a negative control for this experiment, we use the inactive analogue U73343, which lacks efficacy against PLCP. We test if downstream products of PIP₂ hydrolysis alter Nav1.5 currents using the PKC inhibitor staurosporine (1 μM, 1-hour preincubation, Sigma-Aldrich) and the Ca²⁺ chelator, BAPTA (10 mM in the pipette, Sigma-Aldrich).

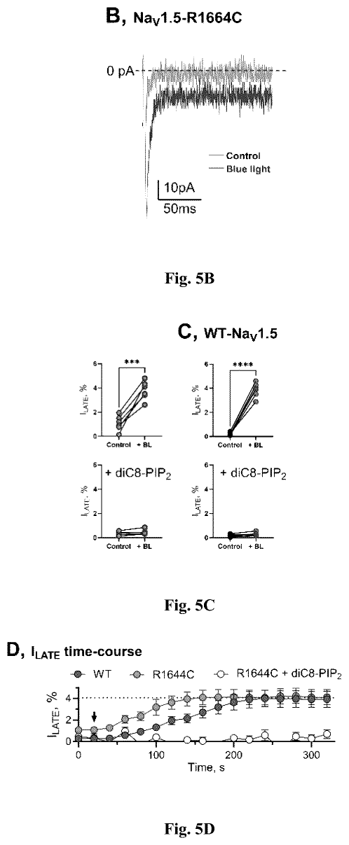

Measure modulation of Nav1.5 by dephosphorylation of PIP₂. In addition to Gαq-signaling, PIP₂ levels are regulated by the activities of specific kinases and PI-phosphatases, which contribute to a dynamic equilibrium that modulates cell signaling and excitable behavior. Hence, it is valuable to test the effects of PIP₂ dephosphorylation (distinct from hydrolysis) on Nav1.5 channels. To study this, we employ the optogenetic tools we have utilized in previous studies. First, we use CRY2-fused pseudojanin (CRY2-PJ), a fusion construct that encompasses the 5-phosphatase inositol polyphosphate 5-phosphatase E and the 4-phosphatase Sacd, depleting PIP₂ by generating PI(4)P and then PI. The CRY2-PJ construct and the CIBN-CAAX membrane anchor are transfected into cardiomyocytes using magnetic transfection (Oz Biosciences). We use the fluorescent protein mCherry as a transfection marker because its spectral properties fall outside the activation range of CRY2. We used this optogenetic system in heterologous cells and cardiomyocytes with simultaneous patch-clamp recording (FIG. 5A). We study Nav1.5 in iPS-CMs using the I-V, SSI and RFI voltage-protocols. Next, we use the I_{LATE} protocol to assess the rate and magnitude of change I_{LATE} when PIP₂ is dephosphorylated. As a control to show that electrophysiological effects correlate to PIP₂ depletion, we study the effect of 200 μM diC8-PIP₂ (Echelon Biosciences) in the recording pipette, a maneuver that opposes the effects of CRY2-PJ on Nav1.4. Further controls are to (1) apply 100 nM Ang II after the completion of the optogenetic study to confirm that Ang II has no effect if membrane pools of PIP₂ are fully dephosphorylated to PI (not a PLCβ substrate), and (2), test photostimulation of CRY2-PJ in the presence of the AT1 receptor antagonist, losartan. This maneuver should not alter the activity of CRY2-PJ. Under physiological conditions dephosphorylation of PIP₂ occurs primarily via the action of inositol 5-phosphatases, generating PI(4)P. To study the effects of dephosphorylating PIP₂ at the 5-position, we used CRY2-5P_{OCRL}, a well-established optogenetic version of the inositol 5-phosphatase region of Lowe's oculocerebrorenal protein, OCRL. Our data on Nav1.4 channels show that CRY2-5P_{OCRL} and CRY2-PJ are equally effective in decoupling iRFP-PH_{PLCδ1} from the plasma membrane. However, CRY2-PJ evokes a ˜25% larger leftward shift in V½_{ACT} than CRY2-5P_{OCRL}, suggesting that the voltage-dependence of Nav1.4 activation gating is influenced, at least in part, by PI(4)P. Here, we test this effect on Nav1.5 V½_{ACT}, V½_{SSI} and I_{LATE}.

Measure the effects of enriching PIP₂ levels on cardiac sodium current. We have shown that enriching PIP₂ by including 200 μM of the soluble, short chain PIP₂ analogue diC8-PIP₂ in the patch pipette results in a rightward shift in V½_{ACT} of Nav1.4, with no effect on V½_{SSI} Given the differences between Nav1.4 and Nav1.5 in our studies, we test the effect of PIP₂ enrichment on I_{Na} in iPS-CMs. Each experiment is performed with a control pipette solution, and a solution containing 200 μM diC8-PIP₂. In the event of a statistically significant current change, the concentration of diC8-PIP₂ is reduced to 100 μM. The study is replicated in HEK293T cells heterologously expressing Nav1.5 channels. Here, we utilize transfection with polyethyleneimine (PEI, Sigma-Aldrich) to express the human Nav1.5 α-subunit along with a GFP-tagged Nav$3l auxiliary subunit. Comparing results between the two systems allows us to determine if a component in the iPS-CM cells modulates the Nav1.5-PIP₂ relationship. In addition to studying diC8-PIP₂, we perform equivalent experiments to test if diC8-variants of other phosphoinositides can modulate Nav1.5 in iPS-CMs and heterologous cells. The most prevalent minor species is PI(4)P, which is a substrate to generate PIP₂. We also test diC8-PI(5)P. Together, these data improve our understanding of which phosphates in the PIP₂ headgroup are required for each aspect of Nav1.5 gating. In addition to whole-cell experiments, we also study sodium channel currents in off-cell, inside out-patches. These experiments allow direct application of PIP₂ to the cytoplasmic face of Nav1.5 channels via macroscopic patches, and in small patches to study single channel behavior. For these experiments, the recording pipette is filled with the external solution and the inside surface of the patch is perfused with intracellular solution. While we anticipate that our recording solutions will isolate single Nav1.5 channels in iPS-CMs, we also collect data from Nav1.5 channels heterologously expressed in HEK293T cells. After establishing the baseline activity of the channel(s), we deplete endogenous PIP₂ from the patch using poly-L-lysine (20 μg/mL). Next, we reintroduce diC8-PIP₂ at increasing concentrations to establish the efficacy and potency of rescue. In addition to testing diC8-PIP₂ and variants, such as diC8-PI4P, these experiments allow us to apply full-length PIP₂. This final experiment tests if the 8-carbon chains of diC8-PIP₂ are sufficient for full rescue and allows us to compare the biophysical effects of the soluble analogue with the native phospholipid.

Native Gαq-induced PIP₂ hydrolysis acutely regulates Nav1.5 gating in iPS-CMs. This mode of regulation is separate from downstream DAG and IP3 signaling, which have distinct biophysical effects on the channel and operate on a longer time scale. In addition to PIP₂ hydrolysis, we test the effects of PIP₂ dephosphorylation on Nav1.5. Based on our data, dephosphorylating PIP₂ has similar biophysical effects on Nav1.5 as hydrolysis of PIP₂. These studies are complemented by experiments to test if exogenous PIP₂ (full length and diC8) can rescue gating dysregulation and increased I_{LATE} associated with PIP₂ depletion, showing if these effects are reversible. Together, this data shows that PIP₂ regulates physiological gating of Nav1.5 channels in cardiomyocytes.

We observe changes in I_{Na} and I_{LATE} with the application of Ang II to iPS-CMs. Notably, the I_{LATE} experiment provides time-resolved changes in response to Ang II. The hydrolysis of PIP₂ is tested using TIRFM to study the rate and the extent at which iRFP-PH_{PLCδ1} transfected into iPS-CMs dissociates from the plasma membrane. Determining this rate allows us to compare the extent of PIP₂ hydrolysis to that evoked by the overexpressed M3q receptor and also support our analysis of the concentration-dependent effects of Ang II in our system. If the effects of Ang II prove too subtle to discern or are inconsistent between cells, we test ET, which signals via the Gαq-coupled ET_{A} receptor in human iPS-CMs.

### Example 2: Nav1.5 Channelopathies Alter PIP₂ Modulation of Cardiac Sodium Current

Nav1.5 channel structure: Nav1.5 channels are composed of a pore-forming α-subunit, encoded by the SCN5A gene, which associates with one or two R subunits. The α-subunit has four homologous domains (DI-DIV), each containing six transmembrane α-helical segments (S1-S6) that incorporate an asymmetric loop located between S5 and S6. This loop lines the inner channel pore and forms the activation gate. Within each domain, S1-S4 constitute the voltage-sensing domain (VSD), with S4 carrying voltage sensing residues, typically positively charged arginines in every third position, which move in response to depolarization to gate the channel. Domains I-III primarily contribute to the activation process, while DIII and DIV, along with the DIII-DIV linker, play a critical role in fast inactivation mechanisms. The current available Nav channel structures have been solved in the absence of PIP₂. Consequently, there is a lack of structural data to indicate where, or how many PIP₂ bind to the Nav1.5 channel. Leveraging the cryo-EM structures of rat and human channels, we have developed molecular models to predict PIP₂ binding pockets and are conducting dynamic simulations to assess how PIP₂ modulates channel gating and ion permeation.

Proarrhythmic Nav1.5 mutations and PIP₂: Uniprot catalogs 429 reviewed variants for LQT3 or BrS that are mapped across the protein and this list continues to expand. Nav1.5 channelopathies manifest a variety of changes in voltage-dependent gating behavior that often result in elevated I_{LATE}. In healthy hearts, I_{LATE} is typically less than 0.5% of the magnitude of peak I_{Na} and is carried by a small number of Nav1.5 channels that reopen after fast inactivation. In patients with ischemic heart disease, sudden infant death syndrome polymorphisms, LQT3 or BrS, I_{LATE} can be elevated to 1-5% of peak I_{Na}. Indeed, increases in I_{LATE} of even 0.3-1% can predispose to sudden cardiac death. Often, symptoms remain subclinical for prolonged periods and the triggers that initiate the disease phenotype are unclear. Changes in PIP₂-channel interaction might directly, or allosterically play a role in the dysregulation of at least some Nav1.5 channelopathies. Existing structural data for the PIP₂-regulated channel Kir2.2, Kv7.1, and Cav2.2, along with extensive mutagenesis studies, demonstrate that PIP₂ is coordinated by positively charged residues that form a local binding pocket for the headgroup within the protein complex. This hypothesis is rational based on electrostatics of the head group with −4 charges at neutral pH. As a result, we hypothesized that Nav1.5 would exhibit a measurable binding affinity for PIP₂ and sought to develop a microscale thermophoresis (MST) approach to quantify it for wild-type and mutant channels. A reduced binding affinity for PIP₂ would render the phospholipid more liable and prone to dysregulation from Gαq signaling or the action of PI-phosphatases.

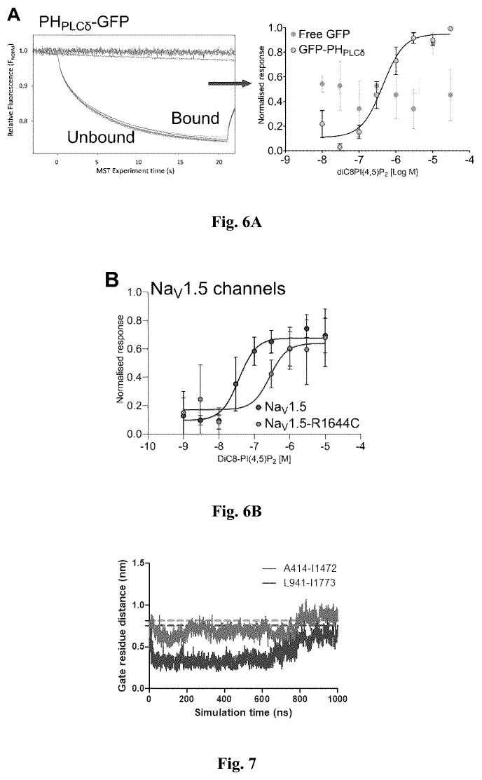

Results. Our studies were designed to evaluate PIP₂-regulation for Nav1.5 channelopathies from multiple perspectives. Given that there is a lack of structural data to identify where, or how many PIP₂ molecules bind to Nav1.5, we performed a docking simulation to identify putative PIP₂ binding sites in the open, rat and human Nav1.5 cryoEM structures (PDB: 6UZ3 and 7DTC) (56-58). We observed four potential PIP₂ binding sites at the boundary between the inner leaflet of the membrane and the cytoplasm with one at the interface between each voltage sensor and the channel body. In each case, we noted electrostatic and hydrogen bond interactions between the headgroup of PIP₂ and multiple disease associated residues. Among these, we elected to perform functional studies on Nav1.5-R1644C, an LQT3 mutant located between the S4 and S5 transmembrane segments of DIV. We expressed wild type Nav1.5 or Nav1.5-R1644C with Nav1β, CIBN-CAAX and CRY2-5P_{OCRL} in HEK293T cells and studied I_{LATE} with and without optogenetic dephosphorylation of PIP₂. In wild-type Nav1.5, I_{LATE} was 0.25±0.1% of the peak (n=8) whereas in Nav1.5-R1664C, I_{LATE} was 1.2±0.2% of peak (n=8, P<0.01, unpaired t-test comparison to WT). Dephosphorylation of PIP₂ elevated I_{LATE} in both channels to ˜4% of the peak but did so more rapidly for Nav1.5-R1644C. Thus, WT Nav1.5 I_{LATE} plateaued after 180±15 s of optogenetic dephosphorylation compared to 86±22 s in Nav1.5-R1644C channels (n=8 for both experiments). These results show that the mutation reduces PIP₂ interactions with the channel. Notably, inclusion of 200 μM diC8-PIP₂ suppressed I_{LATE} in Nav1.5-R1644C channels from 1.2±0.2 to 0.46±0.1% of peak I_{Na} (n=8, P<0.01, unpaired t test), and opposed the elevation in I_{LATE} observed following optogenetic stimulation of CRY2-5P_{OCRL} (FIGS. 5B-5D). Next, we used the Monolith Nanotemper system to establish a microscale thermophoresis (MST) approach to measure the binding affinity of PIP₂ for Nav1.5 channels. MST measures the mobility of the GFP-tagged protein in response to an IR-laser pulse. Ligand binding alters the motion of the target protein, allowing the user to generate a binding curve and extracting a binding constant (Kd) (FIG. 6A). First, we studied GFP-Nav1.5 expressed in HEK293T cells along with Navβ1. Wild type GFP-tagged Nav1.5 displayed a Kd of diC8-PIP₂ at 45±10 nM (n=12 replicates of the experiment). In contrast, the Kd of GFP-Nav1.5-R1644C was ˜8 fold lower (345±22 nM, n=10 replicates of the experiment). We performed additional controls, measuring the Kd of GFP-tagged PH_{PLCδ1} at 130 nM, and free GFP exhibited a value surpassing 800 μM, indicating no binding affinity (FIGS. 6A, 6B).

We conducted all-atom molecular dynamics (MD) simulations on the Nav1.5 channel based on the cryo-EM structures (Rat, PDB: 6UZ3; human, PDB: 7DTC) in an explicit lipid bilayer consisting of POPC, POPE, POPS, and cholesterol at a molecular ratio of 25:5:5:1 in a water box with 150 mM NaCl. A −200 mV transmembrane potential (from the intracellular to the extracellular side) was applied during 1 μs long simulations. In two distinct simulations, we observed two and three Na⁺ permeation events (passing through the selectivity filter to the extracellular side) for the human and rat Nav1.5 channel systems, respectively. FIG. 7 illustrates the changes in distances between gate residues of the channel over the course of simulation time. The distances between gate residues decreased from the initial cryo-EM structure but increased during the last 200 ns of simulations. While no Na⁺ ions permeated through the gate during the first 1 μs of simulations, perhaps due to the decreased distances, we plan to extend the simulation time (2-5 μs) to observe more ion permeation events.

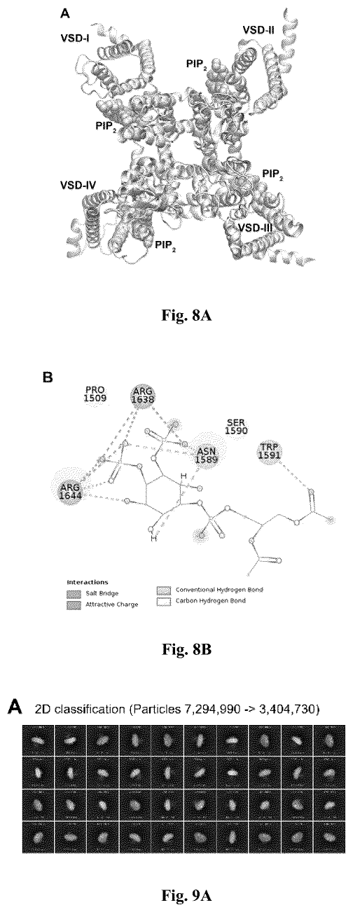

To predict potential PIP₂ binding sites on the Nav1.5 channel, we performed molecular docking simulations of diC1 (a tail-truncated version of PIP₂) against the hNav1.5 structure (PDB:7DTC) using an induced-fit docking (IFD) approach in Schrodinger software. The docking simulations were an unbiased, systematic search covering all possible binding sites for PIP₂ on the channel. Four potential PIP₂ binding sites were identified according to the docking scores (FIG. 8A). The residues forming the predicted PIP₂ binding sites are T238, Q245, K248, K249, W193 (DI); N826, K830, K820, Q779, W781 (DII); N1325, K1479, K1492 (DIII); K1505, R1638, R1644, S1590, N1589, W1591 (DIV). The interacting residues of the binding site in domain IV domain are shown in detail in FIG. 8B. Residues R1644 and R1638 were predicted to form strong salt bridge interactions with PIP₂.

Experimental Design. Our data indicate that the LQT3 disease mutant, Nav1.5-R1644C resides in a putative PIP₂ site and has a significantly reduced affinity for PIP₂. Our electrophysiological studies suggest that the PIP₂ associated with this mutant is more susceptible to dephosphorylation. Consequently, the rate of increase in I_{LATE} is more rapid than for wild type Nav1.5 channels. It is noteworthy that the I_{LATE} of Nav1.5-R1644C is ameliorated when we enrich PIP₂ by introducing a supraphysiological concentration of diC8-PIP₂ in the patch pipette. Building on these insights, we propose experiments to study whether other Nav1.5 channelopathies with enhanced I_{LATE} exhibit reduced affinity to PIP₂, and whether their functional phenotypes can be rescued by enriching PIP₂. Additionally, leveraging our docking model for Nav1.5-PIP₂ interactions, we conduct MD simulations to investigate whether PIP₂ modulates ion permeation and channel gating parameters. This aims to predict all the residues in the channel that are crucial for PIP₂ activity.

Measure the currents in Nav1.5 channelopathies and PIP₂ binding mutants. Nav1.5 mutants will be generated through site directed mutagenesis (Quikchange) and confirmed by sequencing. The GFP-tagged channel constructs are transfected into HEK293T cells along with Navβ1 and their biophysical properties and I_{LATE} characteristics are studied in whole-cell patch-clamp experiments using the recording protocols.

We make and study mutants from three categories:
i) Clinically relevant mutants that our docking model predicts interact with PIP₂. These include Q245(K) in DI, N132(S) in DIII, and K1505(N) in DIV.
ii) Clinically relevant mutants that are not predicted to interact directly with PIP₂. These include the prevalent LQT3/BrS mutants F1759A at the N-terminal end of DIV, S6; E1784(K) at the C-terminal end of DIV, S6 and ΔKPN (Δ1507-1509).
iii) Mutations predicted to coordinate PIP₂ that have not yet been associated with human disease. These include K248 and K249 in DI, K820 and K830 in DII, K1492 in DIII, R1638 in DIV. In each case, the corresponding residue is substituted with a Q. Several mutants of interest have been previously documented, and our results are compared to existing literature. Once the mutants are characterized, we study their functional interactions with PIP₂ using complementary protocols. Initially, we co-express the optogenetic tool CRY2-PJ (with CIBN-CAAX) to assess the impact of PIP₂ dephosphorylation on PIP₂ function. An unstable channel PIP₂ interaction is anticipated to result in an accelerated rate of I_{LATE} elevation from the baseline of the mutant. Profoundly compromised PIP₂ interactions may lead to minimal effects of PIP₂ hydrolysis of dephosphorylation on the channel function. All whole cell studies are compared to inside-out patch studies for PIP₂ apparent affinity and efficacy and MST studies for binding affinity. These experiments are elaborated below. Additional mutants could be added to this experimental scheme based on further predictions from our molecular docking and dynamics experiments, also described below.

Measure Nav1.5 currents after PIP₂ rescue of specific cardiac sodium channelopathies. We study the effect of enriching PIP₂ on the mutants described above, by including 200 μM diC8-PIP₂ in the pipette and performing whole-cell experiments. This diC8-PIP₂ concentration effectively countered the dysregulated gating, abolishing I_{LATE} in the Nav1.5-R1644C mutant during our preliminary studies. However, if a statistically significant change in the current is observed, the concentration of diC8-PIP₂ is reduced to 100 μM. In addition to studying diC8-PIP₂ in whole-cell experiments, we perform equivalent experiments in off-cell patches, using recording conditions. First, we deplete endogenous PIP₂ from off-cell patches using poly-L-lysine (20 μg/mL). Next, we reintroduce diC8-PIP₂ at increasing concentrations to establish the efficacy and potency of rescue. In addition to testing diC8-PIP₂, we study full length PIP₂. This experiment tests if any aspects of the mutant's phenotype are opposed only when the arachidonic and stearyl chains of native PIP₂ are present.

Measure the affinity and efficacy of PIP₂ to modulate Nav1.5 channel variants. The mutants described above are also studied by MST to establish their PIP₂ binding-affinity and in off cell patches to assess efficacy and apparent affinity. We perform MST using the Monolith Nanotemper instrument. This instrument can process 24, 10 μl samples simultaneously to obtain affinity constants in the 1 μM to mM range for GFP-tagged proteins in cell lysates. Cells expressing the GFP-tagged protein of interest is lysed in a PBS buffer containing EDTA, DDM and PMSF then pelleted by centrifugation. After discarding the supernatant, the cells are resuspended in lysis buffer and centrifuged at 15,000 g for 20 minutes at 4° C. After this step, the soluble channel protein is in the supernatant. The samples are aliquoted and mixed with dilutions of diC8-PIP₂ before being loaded into capillaries for the assay. The assays are also run with diC8-PIP₂. In each experiment we will include controls: GFP-tagged PH_{PLCδ1} is a positive control for diC8-PIP₂ binding and has a Kd of 130 nM in our hands and free GFP is a negative control that does not bind diC8-PIP₂ (Kd at >800 μM) (FIG. 6). As ligand binding does not necessarily correlate with a change in channel function, the MST and off cell patch data will be utilized to measure the apparent affinity for diC8-PIP₂ and full length PIP₂ to establish a more comprehensive model of the role of PIP₂ in the function of the wild type and mutant Nav1.5 channels.

Develop a predictive dynamic model of Nav1.5-PIP₂ interactions. Molecular modeling based on existing structures is a powerful tool to probe the dynamics of ion channels. We use this approach to probe the fundamental role of PIP₂ in Nav1.5 channel function, including pore stability and ion conduction. To test whether predicted PIP₂ binding sites are functionally relevant, we conduct MD simulations on Nav1.5-Apo, Nav1.5-1PIP₂ (one site/per simulation system), Nav1.5-4PIP₂ (four sites bound with four PIP₂ molecules), and Nav1.5-mutants in the presence or absence of PIP₂. This methodology mirrors that demonstrated in the results and previously published works. The protonation states of titratable residues of the channel are determined using the H++ server. The channel structures are immersed in an explicit lipid bilayer of POPC, POPE, POPS, and cholesterol with molecular ratio of 25:5:5:1, and a water box with dimensions of around 145.3 Å×145.3 Å×132.2 Å (based on hNav1.5-Apo system) by using the CHARMM-GUI Membrane Builder webserver. Each system is supplemented with 150 mM NaCl and neutralizing counter ions as necessary. Parameter and coordinate files are generated using the tleap (AMBER) program, employing the ff14SB and Lipid17 force fields for proteins and lipids, respectively. MD simulations are conducted using the PMEMD.CUDA program in AMBER 20 under periodic boundary conditions, maintaining isothermal-isobaric ensembles. Long-range electrostatics are computed using the particle mesh Ewald (PME) method with a 10 Å cutoff. Prior to production runs, energy minimizations are conducted. The system is then gradually heated from 0 K to 303 K using Langevin dynamics with a collision frequency of 1 μs-1. Throughout heating, the structures will be restricted in position with an initial constant force of 500 kcal/mol/Å², gradually diminishing to 10 kcal/mol/Å² to afford lipid and water molecules freedom of motion. Subsequently, the systems undergo 5 ns equilibrium MD simulations. Ultimately, 2-5 μs production MD simulations are executed, saving coordinates every 100 μs for subsequent analysis. GROMCAS analysis tools are employed to analyze the trajectories.

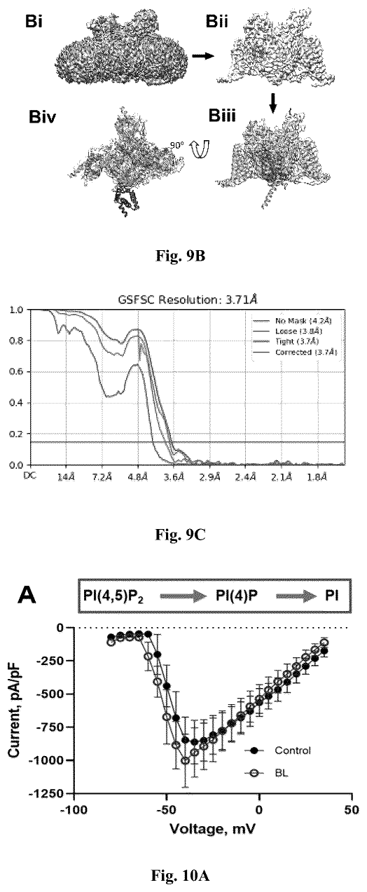

The experiments are designed to fill gaps in our knowledge about the role of PIP₂ in the mechanism of at least certain Nav1.5 channelopathies. Our approach is designed to correlate the function of Nav1.5 channelopathies with reduced PIP₂ interactions. We assess this in four complementary ways: Whole-cell currents, including PIP₂-depletion time course for elevation of I_{LATE}, MST for PIP₂ binding affinity, off-cell patch for efficacy and apparent affinity, and computationally as predictions of likely PIP₂ binding sites and interactions with Nav1.5. This strategy advances our understanding of the mechanisms underlying some channelopathies and if the effects of PIP₂ are direct, allosteric, or unrelated to the disease phenotype. For instance, we anticipate that the common disease mutant E1784K, in the proximal C-terminus do not form a direct interaction with PIP₂. However, it has been demonstrated to interact with a partner residue, K1493 which sits next to a K1492 that might interact directly with the PIP₂ headgroup. We have successfully obtained a Nav1.5 structure at 3.7 Å resolution (FIG. 9).

### Example 3: PI(4,5)P₂ is a Negative Modulator of Cardiac Sodium Channels

### Materials and Methods

Naᵥ1.5 channels were studied with Navβ1 (SCNIB, isoform b; NM_001037) in pRAT, a CMV driven vector. N-terminal CRY2-tagged pseudojanin in pMaX(+) vector was generated by Genscript (Piscataway, NY). Purified diC8PI(4,5)P₂ was purchased from Echelon Biosciences.

### Cell Culture and Transfection

HEK293-T cells were purchased from American Type Culture Collection (ATCC, catalog no. CRL-3216). The cells were cultured in 60-mm dishes in Dulbecco's Modified Eagle Medium (DMEM) (Sigma-Aldrich) that was supplemented with a combination of 100 units/ml of penicillin, 100 μg/ml of streptomycin and 10% Fetal Bovine Serum (FBS). Stable cells were cultured in previously mentioned DMEM, penicillin, streptomycin, and FBS medium with addition of 2 μg/ml puromycin, 5 μg/ml blasticidin, 200 μg/ml geneticin (G418). Both cell lines were incubated at 5% CO2 at 37° C. 24 hours before transfection cells from either cell lines were seeded on glass coverslips in 35-mm dishes. Both cell lines were transfected in OptiMEM using polyethyleneimine (PEI) at a 1 μg of DNA: 4 μL PEI to 1 μg of DNA: 6 μL PEI ratio for 2-4 hours. HEK293-T cells were transfected with 1.5 μg of channel DNA and 1 μg of the beta subunit alone and alongside light-activated constructs. Stable cells were transfected with 1p g of the beta subunit alone and alongside light-activated constructs. Electrophysiology recordings were performed within 24-48 hours after transfection at room temperature.

### Whole-cell Patch Clamp

Both the HEK293-T, and Naᵥ1.5 stable cells were studied under whole-cell patch clamp. Sodium currents were recorded using the Tecella Pico-2 amplifier (Tecella) and WinWCP software (University of Strathclyde) at a low pass (Bessel) filter of 9 kHz and sampled at 50 kHz frequencies. The borosilicate glass (Clark Kent) patch pipettes were pulled to a resistance of 2-4 MQ using the vertical puller (Narishige) when filled with internal solution. The internal solution contained (in mM): 60 CsCl, 80 CsF, 2 MgCl₂·6H₂O, 10 EGTA, 5 HEPES, 5 Na₂ATP, pH 7.4 with CsOH. Cells were perfused through a multichannel gravity-driven perfusion manifold (Warner) with an extracellular solution containing (in mM): 130 NaCl, 5 CsCl, 1.2 MgCl₂·6H₂O, 1.5 CaCl₂), 8 Glucose, 10 HEPES. Cells with a series resistance less than 15 MQ were studied.

Current-voltage relationships were evoked by a 250-ms test pulse between −80 mV to 35 mV, in 5 mV increments. Steady-state inactivation was studied by holding cells at −120 mV and then comparing currents that were evoked by a 50 ms test pulse between −120 mV and −20 mV to those evoked by a 500 ms pre-pulse to 30 mV, in 10 mV increments. Normalized peak currents were then plotted against pre-pulse potential (mV). To determine voltage-dependence, the data was normalized and plotted against the driving force and fitted to a Boltzmann function, I=Iₘₐₓ(1+exp[V−V_{1/2}/k]), where Iₘₐₓ is maximum current and k is the slope factor to generate normalized conductance-voltage (G-V) relationship. The I_{LATE} was evoked by a single test pulse between −90 mV and −20 mV. I_{LATE} was calculated from the last 10 ms of this 250 ms test pulse. From this recording protocol, the time constant for inactivation (T) was determined from a mono-exponential fit of normalized current amplitude using Iₜ=Iₘₐₓ+A [e^{-t/τ}], A is amplitude and t is time. All whole-cell currents were normalized to the capacitance of cell.

### Optogenetics Experiments

Experiments were set up similar to those in Gada et al (2023). Optogenetic studies were conducted simultaneously alongside patch-clamp. Here the photoactivation of cells was conducted in the epifluorescence-mode to utilize continuous excitation from a broad-spectrum LED (Excelitas) through a 448/20 nm filter (Chroma), via a 20× objective lens (Olympus). A photometer (ThorLabs) was used to measure the light output which measured at 50 mW/cm². Prior to patch-clamp studies, cells were kept in the dark to prevent pre-activation of the CRY2-fusion. mCherry was as a marker to identify cells of interest given its spectral properties lie outside the range of CRY2. In addition, we utilized a transilluminator which includes a bandpass filter to obstruct blue light while visualizing cells in the brightfield. The entirety of the experiment was conducted in the dark. In a paired data fashion, cells were recorded before and after blue light illumination. The experiments were all conducted at room temperature.

### Statistics

All patch-clamp data were processed in WinWCP, Clampfit, and Excel software. Statistical analysis and figure generation was conducted in Graphpad (Prism). Except where otherwise stated, the data are shown as mean±standard error (s.e.m.), with statistical differences between paired groups established using two-tailed, paired, Students t-test. The significance level was defined by p<0.05.

Dephosphorylation of PI(4,5)P₂ by CRY2-PJ Alters Naᵥ1.5 Channel Gating

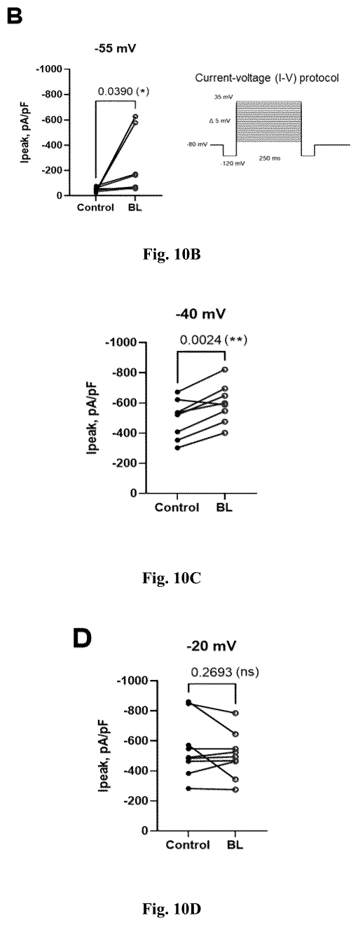

To evaluate the effect of dephosphorylation of PI(4,5)P₂ on the activity of Naᵥ1.5 channels, we combined the optogenetically activated phosphoinositide phosphatase system with whole-cell patch-clamp studies. First, we used the CRY2-PJ to investigate the effect of PI(4,5)P₂ dephosphorylation on Naᵥ1.5 channel activity. CRY2-PJ is recruited to the cell membrane upon BL activation. The CRY2-PJ construct contains in tandem both the 5-phosphatase inositol polyphosphate 5-phosphatase E (INPP5e) and the 4-phosphatase Sac enzymes. Once CRY-PJ is targeted to the cell membrane INPP5e dephosphorylates PI(4,5)P₂ to PI(4)P which is then sequentially dephosphorylated to PI. We transfected Naᵥ1.5 stable cells with GFP-Navβ1-subunit, CRY2-PJ, and GFP-CIBN-CAAX constructs 24-48 hours before whole-cell patch clamp experiments. Cells chosen for whole-cell patch recordings were those expressing the GFP fluorophore. All cells were first were studied under control condition, before blue light illumination (BL), and then subsequently with BL in a paired manner (same cell). BL evoked a left shift of ˜5 mV in the current-voltage (I-V) relationship. In contrast to the BL condition, under control conditions Naᵥ1.5 currents first activated at −55 mV at a current density of −45.2±6.7 pA/pF while under BL conditions a robust current density of −294.6±94.2 was observed (data are represented as mean±s.e.m, n=12, p<0.05; FIG. 10). At peak current density (˜−40 mV) the current density of the BL condition was 20% larger than the control condition at that potential (FIG. 10C).

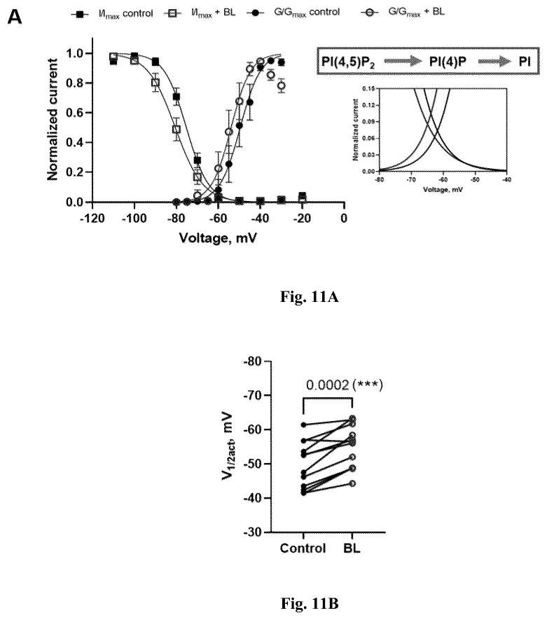

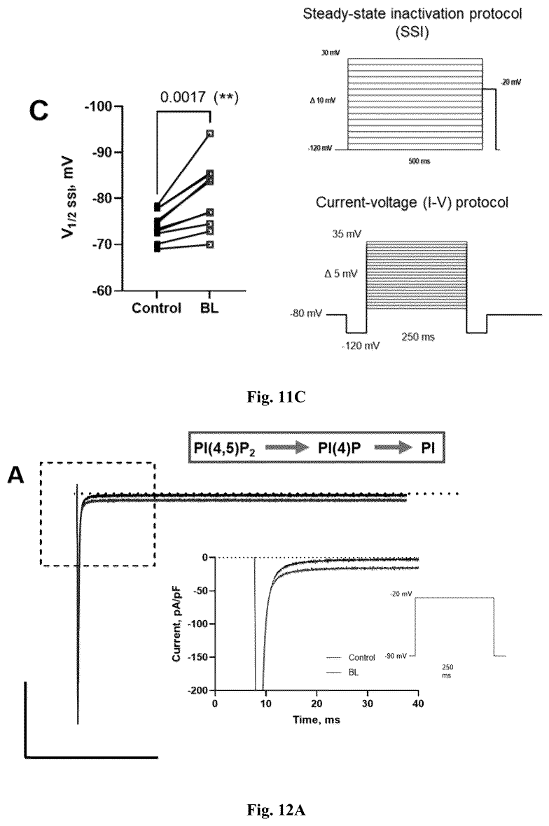

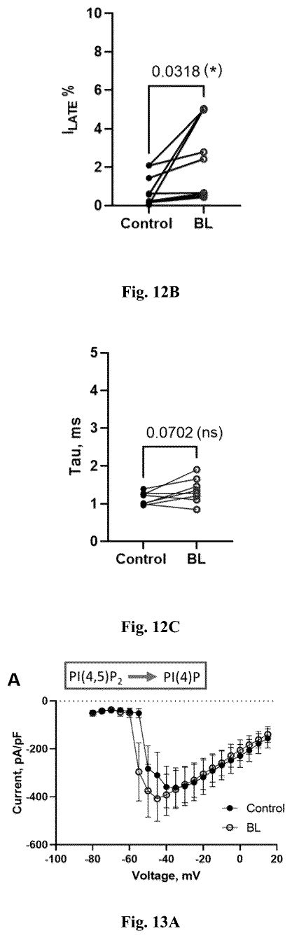

To further investigate the effect of PI(4,5)P₂ dephosphorylation of the voltage-dependence of activation, a conductance-voltage (G-V) relationship was ascertained by replotting the data against the driving force and normalizing and fitting the data with a Boltzmann function. Under the BL condition, the mean half-maximal activation voltage of Naᵥ1.5 (V½_{act}) was −54.9±1.8 mV which is a −5±0.9 mV shift compared to the control condition which was −49.7±2 mV (n=12, p<0.001, paired t-test; FIG. 11B). There was no change in the slope factor of activation (K_{act}). This result differs from what we found in Naᵥ1.4 channels in that, with Naᵥ1.4 channels there is left shift of −10 mV which is twice what we have found in Naᵥ1.5 channels.

We next investigated the effect of dephosphorylating of PI(4,5)P₂ on the voltage-dependence of steady-state inactivation (SSI). SSI was studied by holding cells at −120 mV and then comparing currents that were evoked by a 50 ms test pulse between −120 mV and −20 mV to those evoked by a 500 ms pre-pulse to 30 mV, in 10 mV increments. We found that the mean half-maximal inactivation voltage of Naᵥ1.5 (V½_{SSI}) of the BL condition was −80.40±2.33 mV, which was more hyperpolarized than control condition which was −74.17±1.02 mV (n=10, p<0.01, paired t-test; FIG. 11C). This shift induced by BL activation of CRY2-PJ caused a −5.077±0.13 mV change in the slope factor of SSI (K_{ssI}) compared to the control condition −4.58±0.16 mV (n=10, p<0.05, paired t-test; FIG. 10C). This data indicated that the dynamic voltage range of channel activity was left-shifted in the BL conditions by −6.2±1.41 mV when compared to the control condition. This result is unique to Naᵥ1.5 channels. We found with Naᵥ1.4 channels, photoactivation of CRY2-PJ did not evoke a shift in V½_{SSI} of Naᵥ1.4 channels. However, in both Naᵥ1.4 and Naᵥ1.5 we saw that changes in either V½_{act} or both the V½_{act} and V½_{SSI} produced an increase in the window current (FIG. 11A).

Given the increase in the window current, we evaluated whether dephosphorylation of PI(4,5)P₂ has an effect on the I_{LATE}. An increase in window current indicates that there is an increased number of channels that do not completely inactivate thus continuing to pass Na⁺. As such, there is an increased amount of I_{LATE}. I_{LATE} was studied by evoking a single test pulse between −90 mV and −20 mV over 250-ms. Under the control conditions, the I_{LATE} was 0.83±0.27% while under the BL conditions I_{LATE} was 2.5%±0.69 (Iₚₑₐₖ; n=9, p<0.05, paired t-test; FIG. 12). This result was similar to our results with Naᵥ1.4 channels where we similarly saw I_{LATE} increase to 2%. By fitting the data to the Boltzmann function, we were able to determine the rate of fast inactivation or tau (τ_{inact}). We found that the τ_{inact} of the control conditions to be 1.13±0.06 ms while the BL conditions to be 1.35±0.12 ms (n=8, ns, paired t-test; FIG. 12). This result differs with the robust results that we saw with Naᵥ1.4 channels. In Naᵥ1.4 channels, optogenetic activation of CRY2-PJ increased τ_{inact} from 4.7 ms (control condition) to 9 ms.

Dephosphorylation of PI(4,5)P₂ and CRY25P_{OCRL} Alters Channel Gating

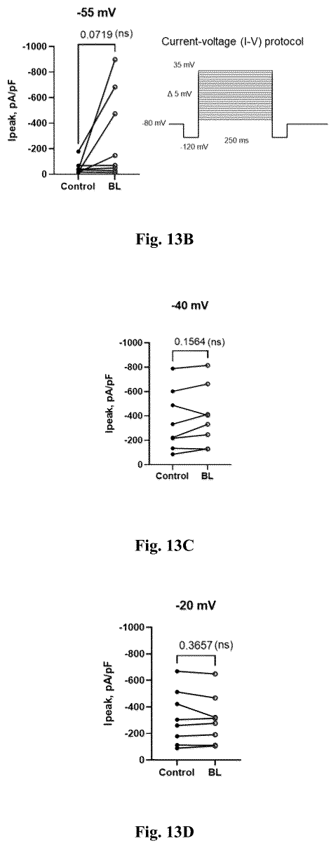

We utilized the CRY2-5P_{OCRL} to investigate the effect of dephosphorylation PI(4,5)P₂ at the 5-position on Naᵥ1.5 channel activity. CRY2-5P_{OCRL} is recruited to the cell membrane upon BL. Once at the plasma membrane CRY2-5P_{OCRL} dephosphorylates PI(4,5)P₂ to PI(4)P. We transfected Naᵥ1.5 stable cells with GFP-Navβ1-subunit, mCherry-CRY2-5P_{OCRL}, and GFP-CIBN-CAAX constructs 24-48 hours before whole-cell patch clamp experiments. Cells chosen for whole-cell patch recordings were those expressing the mCherry fluorophore. All cells were first were studied under control condition, BL, and then subsequently with BL in a paired manner (same cell). BL evoked a left shift of ˜7 mV in the current-voltage (I-V) relationship. In contrast to the BL condition, under control conditions Naᵥ1.5 currents first activated at −55 mV at a current density of −51.06±19.26 pA/pF while under BL conditions at this same potential the current density of −295.58±121.8 mV was observed (n=8, ns; FIG. 13). Similar to what we observed with the Naᵥ1.5-CRY2-PJ and Naᵥ1.5-CRY2-5P_{OCRL}/Sac2 manipulations for dephosphorylating PI(4,5)P₂, BL evokes a left shift in the I-V relationship. As with the Naᵥ1.5-CRY2-5P_{OCRL}/Sac2 this −7 mV causes Nav1.5 currents to activate and peak sodium current density sooner than control conditions. In this case Naᵥ1.5 channels activated as early as ˜−45 mV. When Naᵥ1.5 channels under BL reached ˜−40 mV the current density of the BL condition was 9% larger than the control condition at that potential (FIG. 13). Although the magnitude in the increase of the current density differs slightly what is principally similar is the left shift observed in both these channels.

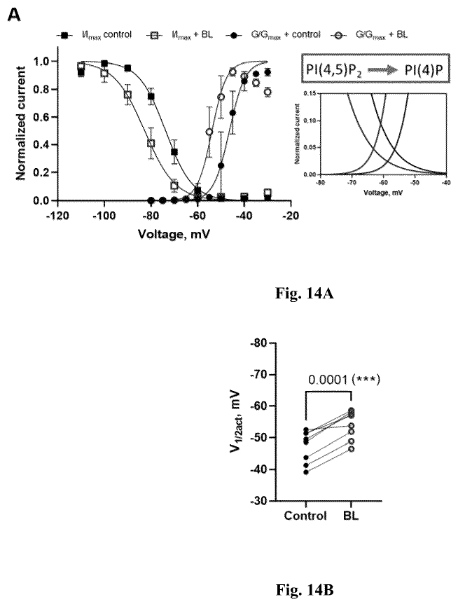

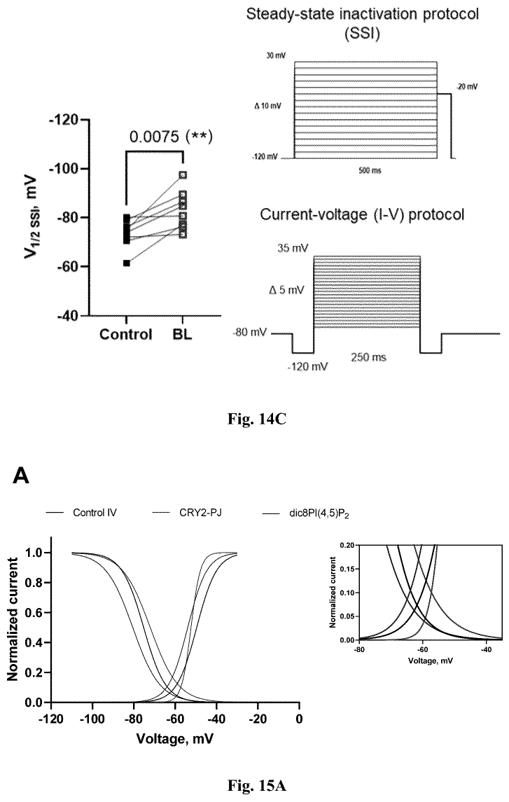

Next, we investigated the effect of PI(4,5)P₂ dephosphorylation on the V½_{act}. Data was obtained as previously mentioned. Under the BL condition, the V½_{act} was −54.05±1.62 mV which is a −7±0.9 mV shift compared to the control condition which was −47.20±1.80 mV (n=8, p<0.001, paired t-test; FIG. 14). The K_{act} changed from 0.93±0.23 mV under the control condition, to 0.53±0.15 mV with BL activation of CRY2-5P_{OCRL}. We found this result to be similar to what we found in Naᵥ1.4 channels. With Naᵥ1.4 channels there was a left shift of −5 mV which is 2 mV less than what we have found in Naᵥ1.5 channels.

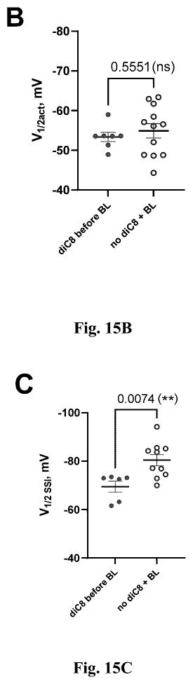

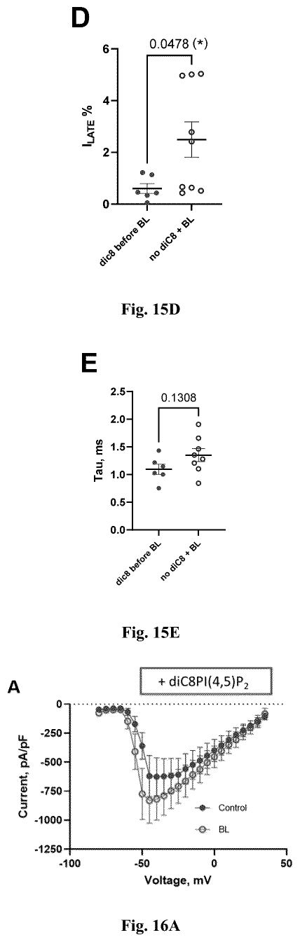

We studied the effect of dephosphorylating of PI(4,5)P₂ on V½_{SSI}. SSI was studied as previously described. We found that the V½_{SSI} of the BL condition was −83.21±2.81 mV, which was more hyperpolarized than control condition which was −73.61±2.07 mV (n=8, p<0.01, paired t-test; FIG. 14). This data indicated that the dynamic voltage range of channel activity was left-shifted in the BL conditions by −9.6±2.58 mV when compared to the control condition. There was no change in the K_{SSI}. Similar to what we saw previously, dephosphorylation of P(4,5)P₂ results in a left shift in the V½_{SSI} which is unique to Naᵥ1.5 channels. Interestingly, the shift in this case was more robust than the last two manipulations. With Naᵥ1.4 channels, photoactivation of CRY2-5P_{OCRL} did not evoke a shift in V½_{SSI} of Naᵥ1.4 channels. However, in both Naᵥ1.4 and Naᵥ1.5 we saw that changes in either V½_{act} or both the V½_{act} and V½_{SSI} produced an increase in the window current (FIG. 15A).

Next, we investigated whether dephosphorylation of PI(4,5)P₂ at the 5-position has an effect on the I_{LATE}. I_{LATE} was studied as previously described. Under the control conditions, the I_{LATE} was 0.62±0.12% while under the BL conditions I_{LATE} was 2.22±0.42% (Iₚₑₐₖ; n=11, p<0.01, paired t-test; FIG. 1C and Table 1). This result was similar to our results with Naᵥ1.4 channels where we similarly saw I_{LATE} increases. We found that the τ_{inact} of the control conditions to be 1.41±0.07 ms while the BL conditions to be 2.58±0.29 ms (n=12, p<0.001, paired t-test; FIG. 15). This is similar to what we observed in with Naᵥ1.4 channels optogenetic activation of CRY2-5P_{OCRL} increased τ_{inact}.

### DiC8PI(4,5)P2 Partially Opposes the Effects of CRY2-5pOCRL/Sac2 Dephosphorylation on Activation and SSI

We sought to determine how a PI(4,5)P₂ enriched environment impacts Naᵥ1.5 channel activity. We accomplished this by including a high concentration of diC8PI(4,5)P₂ in the patch pipette. Naᵥ1.5 stable cells were transfected with GFP-Navβ1-subunit, mCherry-CRY2-5P_{OCRL}, CRY2-Sac2, and GFP-CIBN-CAAX constructs, one to two days before whole-cell patch clamp experiments. Here too the mCherry-fluorophore was used to select cells for whole-cell patch recordings. All cells were studied in a paired manner under control conditions, before BL with 200 μM diC8PI(4,5)P₂, and with BL and 200 μM diC8PI(4,5)P₂. Here, we saw that cells treated with 200 μM diC8PI(4,5)P₂ under BL conditions evoked a small left shift in the I-V relationship (−1.87 mV).

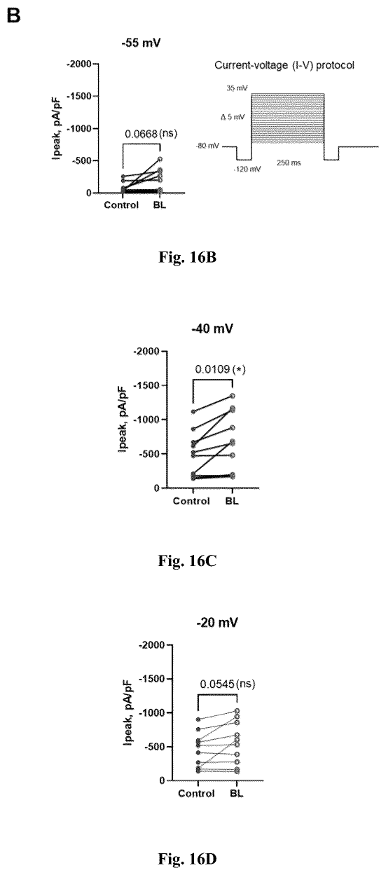

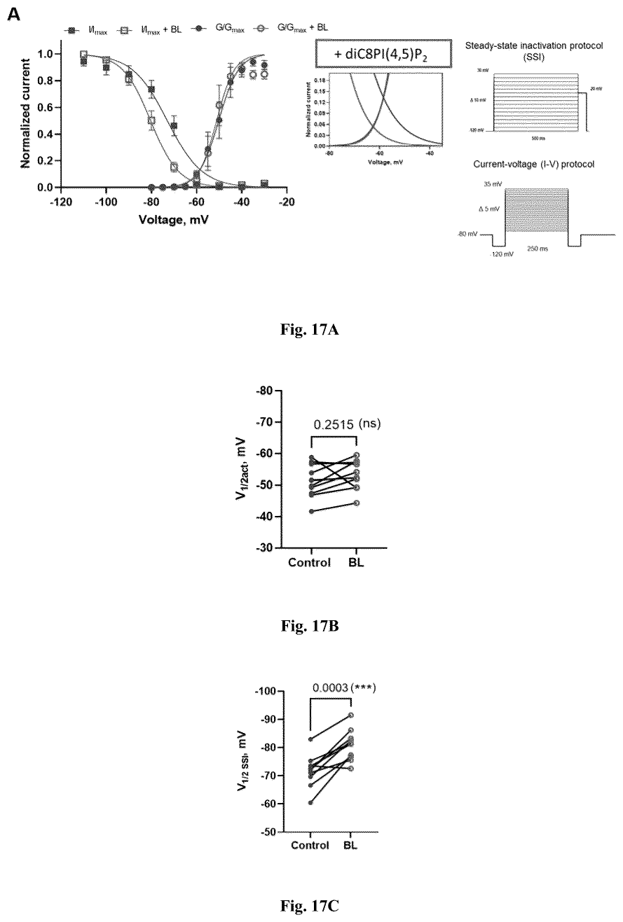

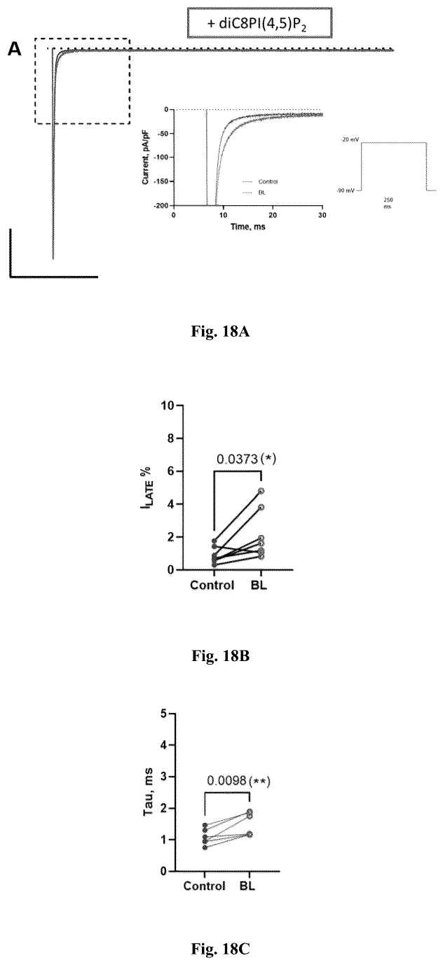

Application of with 200 μM diC8PI(4,5)P₂ to cells under BL conditions caused robust activation of Naᵥ1.5 currents at −55 mV at a current density of −203.83±59.93 pA/pF compared to the control condition of 200 μM diC8PI(4,5)P₂ without BL with a current density of −80.94±28.21 pA/pF was observed (n=9-12, ns). Generally, the current densities of the BL and diC8PI(4,5)P₂ conditions was larger than the diC8PI(4,5)P₂ and no BL condition. Activation of CRY2-PJ in the presence of diC8PI(4,5)P₂ resulted in current densities less than the diC8PI(4,5)P₂ and blue light activation of CRY2-PJ. To interrogate this further we studied the effect of diC8PI(4,5)P₂ enrichment on the voltage dependence of activation. Voltage-dependence of activation was determined. The V½_{act} of cells treated with 200 μM diC8PI(4,5)P₂ under the BL condition was determined to be −53.17±1.49 mV which is a −1.87±1.52 mV shift compared to the control condition which was −51.30±1.7 mV (n=10, ns, paired t-test; FIG. 16C). Comparison of the diC8PI(4,5)P₂ enriched condition (i.e before BL condition) against the BL conditions without diC8PI(4,5)P₂, revealed that a −6.518 mV±2.073 (P<0.01, unpaired t-test) difference in the VV½_{act} between cells. This data provides evidence that diC8PI(4,5)P₂ partially opposes the effects of CRY2-5P dephosphorylation. This is in contrast with the analogous experiment where we compared the effect of dephosphorylation of CRY2-PJ on Nav1.5 in a diC8PI(4,5)P₂ enriched and depleted environment. In these data we found that diC8PI(4,5)P₂ did not oppose the effect of CRY2-PJ phosphorylation on Nav1.5 channels. Here with we see a partial effect by diC8PI(4,5)P₂.

We next investigated the effect diC8PI(4,5)P₂ enrichment the voltage-dependence of SSI. Voltage-dependence of SSI was determined. We found that the the V½_{SSI} of cells treated with 200 μM diC8PI(4,5)P₂ under the BL condition was −80.48±1.75 mV, which was more hyperpolarized than control condition which was −71.75±1.84 mV (n=10, p<0.001, paired t-test; FIG. 16C). This data revealed that the voltage range of channel activity under application of 200 μM diC8PI(4,5)P₂ under the BL condition was left-shifted by −9.1±1.62 mV compared to the control condition. Comparison of the diC8PI(4,5)P₂ enriched condition against the BL conditions without diC8PI(4,5)P₂, revealed a −8.73±2.61 difference in the V½_{SSI} between cells (P<0.01, unpaired t-test). This data provides evidence that diC8PI(4,5)P₂ partially opposes the effects of CRY2-5P_{OCRL}/Sac2 dephosphorylation. This result is supported by the analogous experiment of diC8PI(4,5)P₂ effects of CRY2-PJ dephosphorylation with Naᵥ1.5. Here we also found that diC8PI(4,5)P₂ opposed the effect of the dephosphorylation. Application diC8PI(4,5)P₂ does not oppose changes in window current.

DiC8PI(4,5)P₂ Partially Opposes the Effects of CRY2-5P_{OCRL} Dephosphorylation on SSI

In the previous above study with CRY2-5P_{OCRL} we saw that dephosphorylation of PI(4,5)P₂ resulted in a left shift in the channel's voltage-dependence of both activation and inactivation. As such, we sought to determine how a PI(4,5)P₂ enriched environment would impact Naᵥ1.5 channel activity. Once again, we accomplished this by including a high concentration of diC8) PI(4,5)P₂ in the patch pipette. Naᵥ1.5 stable cells were GFP-Navβ1-subunit, mCherry-CRY2-5P_{OCRL}, and GFP-CIBN-CAAX constructs 24-48 hours before whole-cell patch clamp experiments. Here to the mCherry-fluorophore was used to select cells for whole-cell patch recordings. All cells were studied in a paired manner under control conditions, before BL with 200 μM diC8PI(4,5)P₂, and with BL and 200 μM diC8PI(4,5)P₂. Here too we saw that cells treated with 200 μM diC8PI(4,5)P₂ under BL conditions evoked a left shift of ˜8.61 mV in the I-V relationship.

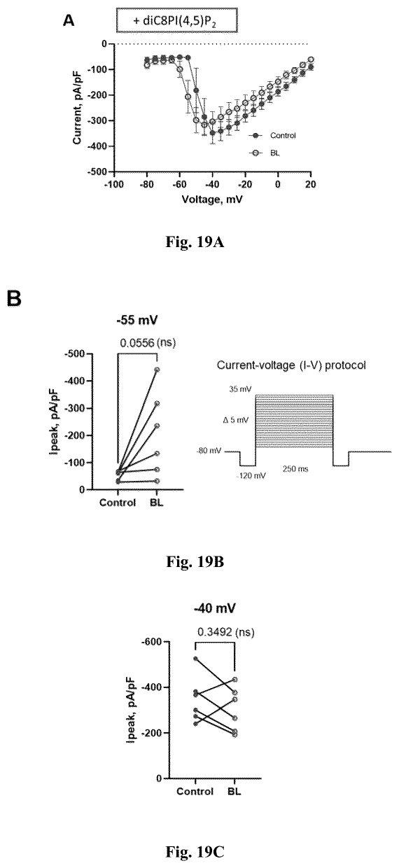

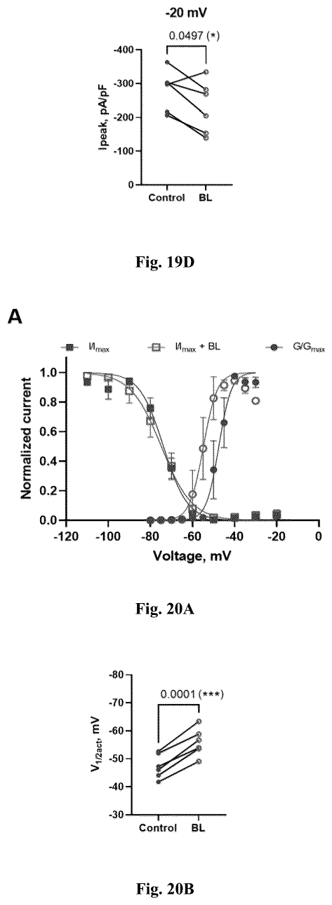

To interrogate this further we studied the effect of diC8PI(4,5)P₂ enrichment on the voltage dependence of activation. Voltage-dependence of activation was determined. The V½_{act} of cells treated with 200 μM diC8PI(4,5)P₂ under the BL condition was determined to be −55.90±2.00 mV which is a −8.61±0.77 mV shift compared to the control condition which was −47.29±1.77 mV (n=6, p<0.001, paired t-test; FIG. 19C). Comparison of the diC8PI(4,5)P₂ enriched condition (i.e. before BL condition) against the BL conditions without diC8PI(4,5)P₂, revealed that a ˜6.768±2.420 mV (P<0.01, unpaired t-test) difference in the V½_{act} between cells. This data provides evidence that diC8PI(4,5)P₂ partially opposes the effects of CRY2-5P dephosphorylation. This contrasts with the analogous experiment of diC8PI(4,5)P₂ effects of CRY2-PJ dephosphorylation with Naᵥ1.5. with the CRY2-PJ where application of diC8PI(4,5)P₂ does not oppose the effect of the dephosphorylation.

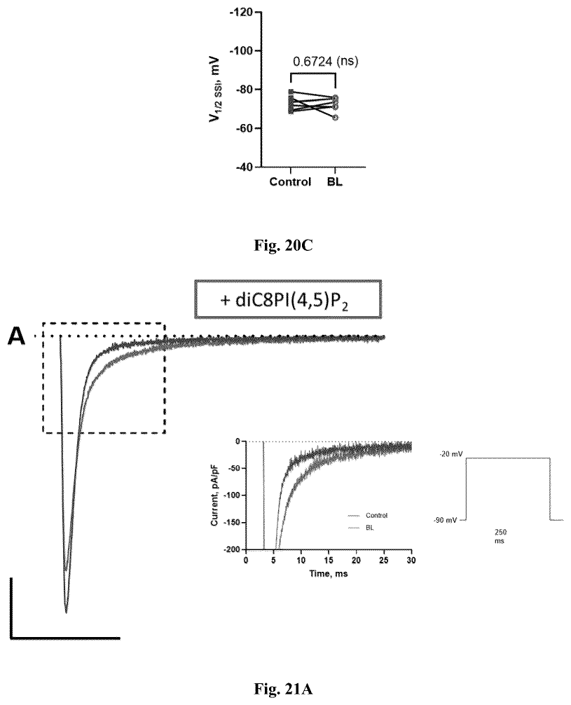

We next investigated the effect diC8PI(4,5)P₂ enrichment the voltage-dependence of SSI. Voltage-dependence of SSI was determined. We found that the V½_{SSI} of cells treated with 200 μM diC8PI(4,5)P₂ under the BL condition was −72.13±1.53 mV, which was more very similar to control condition which was −73.07±1.54 mV (n=6, p<0.0001, paired t-test; FIG. 20C). This data revealed that the voltage range of channel activity under application of 200 μM diC8PI(4,5)P₂ under the BL condition was left-shifted by less than one (−0.9383±2.01 mV) compared to the control condition. Comparison of the diC8PI(4,5)P₂ enriched condition against the BL conditions without diC8PI(4,5)P₂ as in Aim 1.4, revealed a −6.518 mV±2.073 (P<0.01, unpaired t-test) difference in the V½_{SSI} between cells. This data provides evidence that diC8PI(4,5)P₂ partially opposes the effects of CRY2-5P dephosphorylation. This contrasts with the analogous experiment of diC8PI(4,5)P₂ effects of CRY2-PJ dephosphorylation with Naᵥ1.5. with the CRY2-PJ where application of diC8PI(4,5)P₂ does not oppose the effect of the dephosphorylation. Interestingly, in Naᵥ1.4, diC8PI(4,5)P₂ opposes the effects of CRY2-PJ dephosphorylation where in this analogous experiment in Nav1.5 diC8PI(4,5)P₂ does so only partially. Application does not oppose changes in window current. However, the window currents of the PI(4,5)P2 enriched environment is similar to the control condition which is without diC8PI(4,5)P₂ and no blue light activation of the phosphatase.

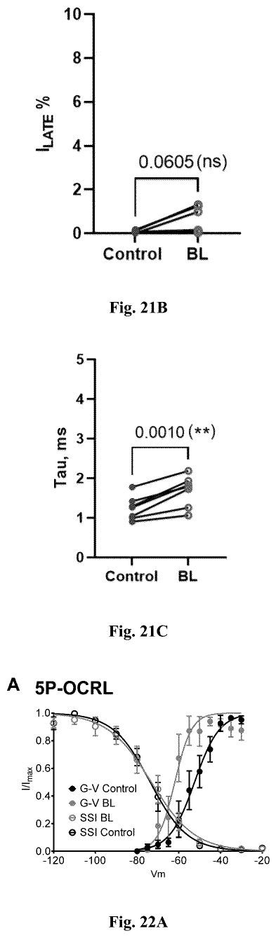

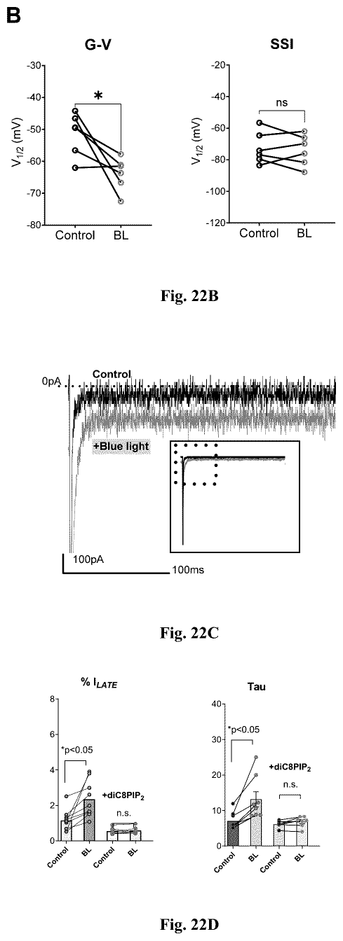

We evaluated whether dephosphorylation application diC8PI(4,5)P₂ oppose the effect of CRY2-5P_{OCRL} dephosphorylation on I_{LATE} and rate of fast inactivation. I_{LATE} and τ_{inact} were determined. Application of diC8PI(4,5)P₂ under BL conditions resulted in an I_{LATE} of 0.64±0.25% compared to under control conditions of 0.08±0.025% (Iₚₑₐₖ; n=6, p<0.05, paired t-test; FIG. 21C). Addition of diC8PI(4,5)P₂ under BL conditions did not oppose increase I_{LATE}. However, comparison of the diC8PI(4,5)P₂ enriched condition against the BL conditions without diC8PI(4,5)P₂, revealed that 2.139±0.5794 (P<0.01, unpaired t-test) difference in the I_{LATE} between cells. We found the τ_{inact} of cells treated with diC8PI(4,5)P₂ under BL conditions to 1.68±0.15 ms while the control condition to be 1.24±0.3 ms (n=6, P<0.01, paired t-test; FIG. 21C). Addition of diC8PI(4,5)P₂ under BL conditions did not slow rate of fast inactivation. When we compared the diC8PI(4,5)P₂ enriched condition against the BL conditions without diC8PI(4,5)P₂ as in aim 3.2 we found a 1.345±0.3918 ms (P<0.01, unpaired t-test) difference in the τinact between cells. These data provide evidence that diC8PI(4,5)P₂ partially reverses the effects of CRY2-5P dephosphorylation.

Next, aimed to test the hypothesis that the relationship of PIP₂ with the channel in certain disease-associated variants is altered. We employed a variant of Nav1.5 identified in patients with Long QT Syndrome Type 3, Arg1644→C and assessed it sensitivity to the widely used 5P-OCRL construct. We observed a right shift in the G-V curve and an increase in the I_{LATE} over WT conditions as well as a further increase following blue light induction of PIP₂ dephosphorylation. We then used the patch pipette to enrich cells expressing these channels with diC8PI(4,5)P₂. We observed that the enhanced I_{LATE} observed under control conditions is mitigated by the inclusion of diC8PIP₂ in patch pipette while the shift in the G-V is not rescued by the inclusion of diC8PI(4,5)P₂ in the pipette.

### Biophysical Assessment of Affinity of Nav1.5 for Phosphoinositides

We next aimed to demonstrate the relationship between Nav1.5 and PI(4,5)P2 through a protein-ligand lens. To this end, we have developed a microscale thermophoresis (MST) assay that can predict the affinity of Nav1.5 channels for PIP2. MST methods for measuring protein-protein, protein-ligand and nucleic acid interactions are amply available. This is the first report of a protein-lipid MST assay that uses cell lysates and does not rely on purified protein such as in surface plasmon resonance. This assay relies on lysates of cell overexpressing a fluorescently tagged target, such as mTFP-Nav1.5 incubated with increasing concentrations of diC8PI(4,5)P₂. We used the pleckstrin homology domain of phospholipase C delta (GFP-PH_{PLCδ}), a PIP₂ biosensor, as a positive control and free GFP as a negative control to demonstrate the affinity of a known PIP₂ binder and non-binder. The affinity of PH_{PLCδ} was measured by this assay. We observed that the affinity of mTFP-Nav1.5(R16444C) was almost ten-fold lower than the WT channel in this assay. This is in line with our observations of an increased I_{LATE} which can be brought down to WT levels following diC8PI(4,5)P₂ enrichment via the patch pipette.

### CONCLUSION

In this study we evaluated the effect of PI(4,5)P₂ dephosphorylation on Naᵥ1.5 channel function before and after optogenetic activation of distinct phosphoinositide enzymes. Sequentially dephosphorylation of PI(4,5)P₂ at the 4 and 5 positions of the inositol ring was conducted using the CRY tagged pseudojanin construct. We concluded that depletion of PI(4,5)P₂ and PI(4)P by photoactivation of the CRY2-PJ construct induced a left shift in the voltage-dependent gating of Naᵥ1.5 towards hyperpolarize membrane potentials, increased late current, and generally increased the rate of fast inactivation. When we evaluated dephosphorylation of PI(4,5)P₂ by photoactivation of only the CRY2-5P_{OCRL} we found similar results to that of CRY2-PJ. The left shift in the voltage-dependence of activation, increase in late sodium current, and increase in the rate of fast inactivation that was observed in these studies, although different in magnitude across the various phosphatase conditions, was comparable to the shift we saw in Naᵥ1.4. This left shift in the conductance-voltage relationship is in line with similar findings in other voltage-gated ion channels. What differed from results with Nav1.4 is that, with Naᵥ1.5 channels, depletion of PI(4,5)P₂ levels evokes a left shift in the steady-state inactivation process. Overall, our results with the optogenetically activated phosphoinositide phosphatases revealed that depletion of PI(4,5)P₂ levels negatively modulates Naᵥ1.5 channel activity. The effect of diC8PI(4,5)P₂ on Naᵥ1.5 channel activity revealed distinct changes that were induced by activation of the different phosphoinositide phosphatases. We found that application of diC8PI(4,5)P₂ does not oppose the effects of CRY2-PJ dephosphorylation. We saw left shifts in the voltage-dependence of activation and inactivation, increase in I_{LATE}, and increase rate of fast inactivation. Comparison of PI(4,5)P₂ rich environment against the PI(4,5)P₂ depleted environment revealed that diC8PI(4,5)P₂ partially opposes the effect CRY2-PJ dephosphorylation on the voltage-dependence of inactivation. When we once again took into consideration of the behavior of channel in a PI(4,5)P₂ enriched environment over a PI(4,5)P₂ depleted environment, we found that di8CPI(4,5)P₂ partially rescues the effect of dephosphorylation of CRY2-5P_{OCRL}/Sac2 on both the voltage-dependence of activation and inactivation. We found that di8CPI(4,5)P₂ rescued the voltage-dependence of inactivation. In this case too we found that di8CPI(4,5)P₂ partially opposes the effect of CRY2-5_{POCRL} in a PI(4,5)P₂ enriched environment.

Although, diC8PI(4,5)P₂ possess a myo-inositol headgroup like in the native PI(4,5)P₂, it differs structurally to the to the native PI(4,5)P₂ where the length of acyl chains is concerned. Rather than the 18 to 20 carbon steric and arachidonoyl acyl chains, diC8PI(4,5)P₂ possess a dioctanoyl chain which it interacts with the membrane. Given this knowledge, it is possible that the length of acyl chains of PI(4,5)P₂ might impact Naᵥ1.5 channel gating behavior. It is also important to note that PI(4,5)P₂ dephosphorylation produces daughter PI-species that may still interact with the channel and block the effects of diC8PI(4,5)P₂.

Long QT type 3 (LQT3) and Brugada syndrome (BrS) are two most prevalent arrhythmias associated with mutations in the SCN5A gene. R1644C is a clinically relevant LQT3 and BrS mixed phenotype mutant that is in the inactivation region of the channel. This mutant has increased late sodium current and altered kinetics of inactivation which spurred us to test whether PI(4,5)P₂ could rescue this pathogenic I_{LATE} current. We concluded that PI(4,5)P₂ was reversed the late sodium current. Further, we show through a novel biophysical method that this variant has decreased affinity for PIP2 compared to the WT channel. This possibility argues for a structural study of Nav1.5-PI(4,5)P2 interactions to evaluate likely binding pockets for this enigmatic phospholipid on the cardiac sodium channel. In conclusion, we provide evidence that dephosphorylation of PI(4,5)P₂ alters Naᵥ1.5 channel gating behavior. Structural insight about the channel-PI(4,5)P₂ interaction will aid in identifying key interaction sites. Furthermore, insight into channel-PI(4,5)P₂ interactions will aid in understanding the role of PI(4,5)P₂ in Naᵥ1.5 channelopathies.

### Example 4: Cell Lysis for MST Experiment

1. Place PBS-EDTA buffer on ice for 10-15 mins before use.
2. Aspirate media from cell culture dish.
3. Add 4 mL of cold PBS-EDTA buffer to a 10 cm dish.
4. Manually dislodge cells and pipette the suspension into a 15 mL conical tube.
5. Centrifuge the suspension for 5 min at max speed.
6. Discard supernatant.
7. Resuspend the cells in 1 mL of co/dPBS-EDTA buffer and transfer to a 1 mL centrifuge tube.
NOTE: Cells can be stored at −80° C. at this stage to be lysed at a different time.
8. Centrifuge the cells using a table-top centrifuge for 1 minute at max speed.
9. Discard supernatant.
10. Reconstitute with 500 μL of lysis buffer and suspend by pipetting on ice for 30 min. (Suspend lysates every 5-10 min by inverting tubes)
11. Centrifuge (>15000×g) at 4° C. for 20 min
12. Take sup. carefully (and aliquot?). Precipitates may be loose.

### Lysis Buffer:

| Com- | | | | For | | For | | |
|---|---|---|---|---|---|---|---|---|
| pound | STOCK | FINAL | | 10 mL | | 5 mL | | |
| PBS | 10X | 1X | | 1 | ml | 0.5 | ml | |
| DDM | 10% | 0.5% | | 0.5 | ml | 0.25 | ml | |
| PMSF | 0.1M | 1 | mM | 100 | ul | 50 | ul | |
| EDTA | 0.5M | 0.5 | mM | 10 | ul | 5 | ul | Add PMSF to |
| | | | | | | | | the buffer just |
| | | | | | | | | before lysing |
| | | | | | | | | cells |

| | | | | |
|---|---|---|---|---|
| H₂O | ~6.4 | ml | ~3.2 | ml |

PBS-EDTA can be found in the 3-door fridge.
diC8PIP₂ highest conc used was 30 uM in diH2O.
diC8PIP₂ concentrations prepared in diH₂O were (in uM) 60, 20, 6, 2, 0.6, 0.2, 0.06, 0.02, 0.006, 0.002, 0 uM.
Add 10 uL sample+10 uL diC8PIP₂ to a 384 well plate.
Add ______ uL of Tween-20** to each tube.
Fill capillaries.
** add tween-80 to enhance sample readability. Especially in the Nav1.5 conditions because it said fluorescence intensity was low.

### Example 5: PI(4,5)P₂ Regulates the Gating of Nav1.4 Channels

### Materials and Methods

### Reagents and Molecular Biology

Purified diC8PI(4,5)P₂ was purchased from Echelon Biosciences. For oocyte studies, plasmid vectors were linearized after the Xenopus β-globulin domain of each construct, verified by gel electrophoresis, and transcribed in vitro using the mMESSAGE mMACHINE T7 Transcription Kit (Thermo Fisher Scientific) to generate cRNA according to the manufacturer's protocol. Rat Nav1.4 (SCN4 Å; NM_013178.2) was handled in pBud, a CMV driven vector. To study Nav1.4 currents, the plasmid was used in concert with Navβ1 (SCN1B, isoform b; NM_001037) in pRAT, a CMV driven vector. The inactivation deficient mutant, Nav1.4-WCW was handled in pCap. CRY2-Sac2 was subcloned into pMAX(+) for expression in mammalian cells, or the generation of cRNA for use in Xenopus oocytes. The open reading frame of pseudojanin was designed with an N-terminal CRY2-tag in the pMAX(+) vector and was generated by Genscript (Piscataway, NY).

### Cell Culture

Human embryonic kidney (HEK293T) cells were acquired from American Type Culture Collection (ATCC, Cat #CRL-3216) and were maintained in Dulbecco's modified Eagle's medium (ATCC) supplemented with 100 units/ml penicillin, 100 μg/ml streptomycin, and 10% (vol/vol) fetal bovine serum. Cells were routinely confirmed to be mycoplasma free via PCR and Hoescht stain. The cells were incubated in a 37° C., humified incubator supplemented with 5% CO2. For our experiments, cells were seeded on glass coverslips in 35 mm culture dishes at least one day before transfection. Cells were transiently transfected with rNav1.4, Navβ1, CIBN-CAAX and CRY2-5-Ptase_{OCRL} or CRY2-pseudojanin, along with the near-infrared PI(4,5)P₂ biosensor iRFP-PH_{PLCδ1} as indicated, in OptiMEM using polyethyleneimine (PEI) for 1-2 hours at a ratio of 1 μg of DNA to 4 μL PEI. The near red fluorescent protein, mCherry, was typically used as a transfection marker. The cells were studied 24-48 hours after transfection.

### Total Internal Reflection Fluorescence Microscopy (TIRFM)

HEK293T cells seeded on 35 mm, glass-bottomed petri dishes were transfected using PEI (1:4) with 1 μg iRFP-PH_{PLCδ1}, and 0.75 μg of CRY2-PJ/CRY2-5P_{OCRL}, with 0.75 μg CIBN-CAAX 48-hrs before the experiments. Blue-light-activation of CRY2 phosphatases was performed using a 445-nm laser and iRFP was excited by a 647-nm laser (OBIS, Coherent, Santa Clara, CA) in an imaging solution comprising (in mM): NaCl 130, KCl 4, MgCl₂ 1.2, CaCl₂) 2, HEPES 10, pH was adjusted to 7.4 with NaOH. Cells were illuminated at 647 nm in TIRF mode to visualize iRFP and at 445 nm to activate the optogenetic machinery. Data were collected at 5-s intervals with a 10 ms exposure time to minimize photobleaching and were saved as stacked TIFF images. Upstream of the laser clean-up filters, the beams were conditioned for coherence using home-made Keplerian beam expanders. A high numerical-aperture apochromatic objective (60×, 1.5 NA; Olympus, Waltham, MA) installed on an RM21 microscope frame with a piezo-driven nano-positioning stage was illuminated by laser lines calibrated to provide 10 mW of incident light (MadCity Labs., Madison, WI) (Gada et al., 2023b). The fluorescence from iRFP was captured using a back illuminated sCMOS camera from Teledyne Photometrics in Tucson, Arizona. The camera and the lasers were controlled using the free software Micro-Manager (UCSF). Tetraspec beads (Thermo, Waltham, MA) were used to calibrate the evanescent field depth to 100 nm and map the sCMOS chip. We used ImageJ to analyze the TIRF data.

### Electrophysiology

Whole-cell patch clamp. HEK293T cells were studied by whole cell patch-clamp. Currents were recorded with a Tecella Pico-2 amplifier (Tecella) controlled using WinWCP software (University of Strathclyde). Currents were low pass (Bessel) filtered at 9 kHz and digitized at 50 kHz. Patch pipettes were pulled from borosilicate glass (Clark Kent), using a vertical puller (Narishige) and had a resistance of 2.5-4 MQ when filled with internal solution containing (in mM): 60 CsCl, 80 CsF, 2 MgCl₂·6H₂O, 10 EGTA, 5 HEPES, 5 Na₂ATP. The cells were perfused using a multichannel gravity-driven perfusion manifold (Warner) with an extracellular solution containing (in mM): 130 NaCl, 5 CsCl, 1.2 MgCl₂·6H₂O, 1.5 CaCl₂), 8 Glucose, 10 HEPES. Data was collected from cells with a series resistance less than 15 MQ. The junction potential was less than 3 mV and was not compensated.

Current-voltage (I-V) relationships were evoked from a holding potential of −90 mV by 400 ms test pulses between −80 and 50 mV, in 5 mV increments. Steady-state inactivation was studied by holding the cells at −140 mV and then comparing currents evoked by 50 ms test pulses between −140 mV and 30 mV to those evoked by a 100 ms prepulse to 0 ms. A 10 s interpulse interval was used in both cases. Normalized peak current values are plotted against the prepulse potential (mV). To quantify voltage-dependence, the data were normalized, plotted against the driving force to generate normalized conductance-voltage relationships (G-V) that were fit using a Boltzmann function, I=I_{MAX}/(1+exp[V−V ½/K]), where I_{MAX} is the maximum current and K is the slope factor. Recovery from fast inactivation was studied by holding cells at −100 mV and comparing currents evoked by a pair of 50 ms test step to −30 mV separated by an interpulse interval that increased in duration by 5 ms increments per sweep. The time constant for recovery from inactivation (τ_{inact}) was obtained from mono-exponential fits of the normalized current amplitude to the recovery time using Iₜ=Iₘₐₓ+A[e^{-t/τ}], where ‘A’ is the amplitude of components and ‘t’ is time. Whole-cell currents were normalized to cell capacitance. Mean±SEM capacitance values were 12±4 pF for HEK293T cells.

For simultaneous optogenetic-patch clamp studies the blue light system was photoactivated in epifluorescence-mode using continuous excitation from a broad-spectrum LED (Excelitas) through a 448/20 nm filter (Chroma), via a 20× objective lens (Olympus). The light output at the sample was measured at 50 mW/cm² by a photometer (ThorLabs). To avoid pre-activation of CRY2-fusion proteins, cells were incubated in the dark and handled in foil wrapped dishes prior to the experiments. Cells of interest were identified using mCherry as a transfection marker because its spectral properties fall beyond the activation range of CRY2. Further, we use a transilluminator that includes a bandpass filter to block blue light during brightfield visualization. To generate paired data, the currents were recorded first in the dark and again following a 3-min pre-illumination with epifluorescent blue-light that persisted through the remainder of the study. All the experiments were performed at room temperature.

Two-electrode voltage clamp. Oocytes were isolated from Xenopus laevis frogs and surgically isolated, dissociated, and defolliculated using collagenase according to standard protocols. Isolated oocytes were transferred to an Oocyte Ringer's solution supplemented with Ca²⁺ and Penicillin/Streptomycin antibiotics. Oocytes were each selected and injected with 50 nL of cRNA resuspended in RNAse-free water. After injection, oocytes were incubated at 17° C. prior to TEVC experiments. Electrodes were pulled from borosilicate glass capillaries using a Flaming-Brown micropipette puller (Sutter Instruments) and filled with a 3M KCl solution containing 1.5% agarose. Pipette resistances were between 0.2 and 1.0 MQ. Currents were recorded two days after injection using a GeneClamp 500 (Molecular Devices) or an OC-725D (Warner) amplifier. Oocytes were perfused with a gravity-driven apparatus with a physiological buffer containing (in mM): KCl 2, NaCl 96, MgCl₂ 1, and HEPES 5, buffered to pH 7.4 with NaOH. Blue light was generated using a 470 nm LED (Luminus) that was focused on the oocytes using a collimating lens, as previously described (Gada et al., 2023c). The incident light was powered to 5 mW/cm², as determined by a photometer (ThorLabs). Currents were evoked from a holding potential of −90 mV by 400 ms step depolarizations between −80 and 50 mV with 5 mV increments and an interpulse interval of 7.5 s. To quantify voltage-dependence, the data were normalized, plotted against the driving force to generate normalized conductance-voltage relationships (G-V) that were fit using a Boltzmann function, I=I_{MAX}/(I+exp[V−V ½ K]), where I_{MAX} is the maximum current and K is the slope factor, was used to fit. To generate paired-data sets, the currents were recorded before and again following a 3-min pre-illumination period with continuous illumination through the remainder of the experiment.

### Statistics

Data were handled in WinWCP, Clampfit, and Excel software with statistical analysis performed using GraphPad (Prism). The data are presented as mean±standard error (s.e.m.) with statistical differences between paired groups determined by two-tailed, paired, Students t-test, unless indicated otherwise. The threshold for significance was determined to be p<0.05. We used Biorender to generate the schematic diagrams.

### Results

### Dephosphorylation of PI(4,5)P₂ Modifies the Gating Parameters of Nav1.4 Channels.

To study the role of PI(4,5)P₂ in the activity of Nav1.4 channels we combined whole-cell patch-clamp recording with photoactivation of membrane targeted PI-phosphatases that dephosphorylate PI(4,5)P₂. This robust optogenetic platform is based on blue light (˜440-490 nm) photoactivation of Cryptochrome 2 (CRY2) and its protein partner, CIBN. Fusing CIBN to the membrane anchor CAAX targets CRY2 and its cargo to the inner leaflet of the cell membrane in response to photostimulation. Prior studies using real-time measurements of PI(4,5)P₂-regulated channel activity, or PI(4,5)P₂ biosensors such as the PH-domain of PLCδ, as reporters have shown that the membrane localization of CRY2-fused PI-phosphatases, and subsequent dephosphorylation of PI(4,5)P₂ occur within tens of seconds of photoactivation. Although PI(4,5)P₂ can be regenerated within minutes, this effect can be mitigated by continued photostimulation.

First, we studied decoupling of the PI(4,5)P₂ biosensor iRFP-PH_{PLCδ1} from the plasma membrane of HEK293T cells following photoactivation of CRY2-fused pseudojanin (CRY2-PJ). Pseudojanin is a fusion construct that encompasses the 5-phosphatase inositol polyphosphate 5-phosphatase E and the 4-phosphatase Sacd, depleting PI(4,5)P₂ by generating PI(4)P and then PI (FIG. 24A). Using total internal reflection fluorescence microscopy (TIRFM) we determined that iRFP-PH_{PLCδ1} decouples from the membrane rapidly in response to blue-light illumination (BL) and completely within ˜225 s (FIGS. 24B, 24C). These findings are consistent with our prior observations and the work of others using iRFP-PH_{PLCδ1}.

Using whole-cell patch-clamp, we studied HEK293T cells expressing rat Nav1.4, Navβ1, CRY2-PJ, and CIBN-CAAX in HEK293T, and compared the currents evoked by a step depolarization protocol before and after photoactivation. Under control conditions, Nav1.4 currents first activated at −50 mV and reached a peak current-density of −312±23 pA/pF at −30 mV (data are mean±s.e.m.). BL-illumination evoked a −10 mV leftward shift in the current-voltage (I-V) relationship, such that robust activation of Nav1.4 channels was first observed at ˜−55 mV. The current-density peaked at −40 mV and was −32% larger than the control at that potential (FIG. 24D and Table 1). To interrogate this effect further, the data were replotted against the driving force, normalized, and fit with a Boltzmann function to obtain a conductance-voltage (G-V) relationship. Photostimulation of CRY2-PJ caused a ˜10±2 mV leftward shift in the mean half-maximal activation voltage of Nav1.4 (V½_{act}) without a change in the slope factor, K_{act} (FIG. 25A and Table 2).

Next, we studied the effect of PI(4,5)P₂ dephosphorylation on the voltage-dependence of steady-state inactivation (SSI) using a paired-pulse protocol, where the prepulse was increasingly depolarized and the test-pulse was of fixed amplitude. Activation of CRY2-PJ did not alter the half-maximal voltage-dependence of SSI (V½_{SSI}) but flattened the slope of the curve (K_{SSI}) such that shifts in SSI were more pronounced when the test potential was more depolarized than V½_{SSI} (FIG. 25A and Table 2). Notably, the changes in V½_{act} and K_{SSI} augmented the magnitude of the ‘window’ current between the voltage-dependent activation and SSI gating processes of Nav1.4 channels (FIG. 25A, inset).

DiC₈PI(4,5)P₂ Opposes the Effects of CRY2-PJ Dephosphorylation.

Given that dephosphorylating PI(4,5)P₂ evoked a left shift in the V½_{act} of Nav1.4 gating we sought to study the channels in a PI(4,5)P₂ enriched environment. To accomplish this, we incorporated a high concentration of the soluble, short chain PI(4,5)P₂ analogue diC8PI(4,5)P₂ in the patch pipette. Treating cells with 200 μM diC8PI(4,5)P₂ evoked a −5.5 mV right-shift in V½_{act} and a ˜3 mV right-shift in V½_{SSI} compared to untreated cells, without changing K_{act} or K_{SSI} (FIGS. 25B, 25C and Table 2). In addition, diC8PI(4,5)P₂ ameliorated the effects of CRY2-PJ photoactivation on V½_{act} and abolished the increase in the magnitude of the window current (FIG. 25B, inset). In total, the change in mean V½_{act} between cells treated with 200 μM diC8PI(4,5)P₂ and control cells studied following dephosphorylation of endogenous PI(4,5)P₂ by CRY2-PJ was −16±2 mV, with a −5.4±2 mV shift in the mean V½_{SSI} (FIG. 25C). Together, these data indicate that the gating parameters of Nav1.4 channels are modulated by the level of PI(4,5)P₂ in the membrane.

DiC₈PI(4,5)P₂ Opposes the Effects of CRY2-5P_{OCRL} Dephosphorylation.

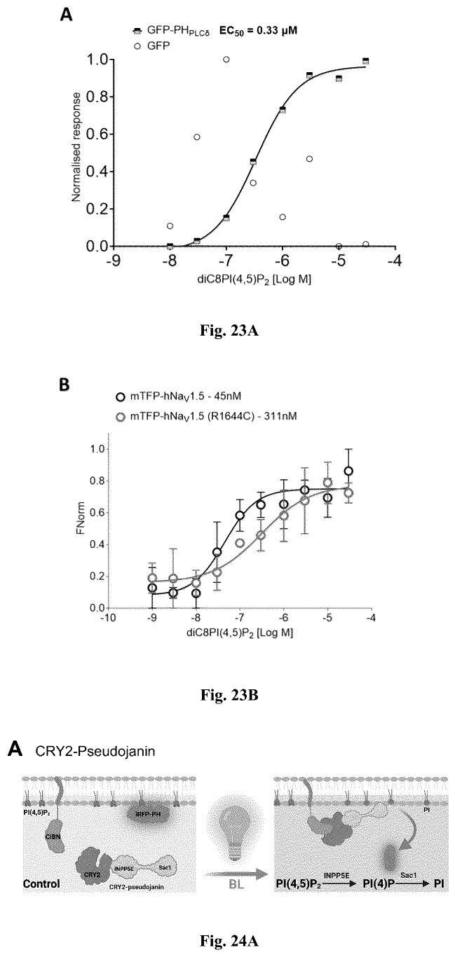

Under physiological conditions dephosphorylation of PI(4,5)P₂ occurs primarily via the action of inositol 5-phosphatases, generating PI(4)P. To study the effects of dephosphorylating PI(4,5)P₂ at the 5-position, we used CRY2-5P_{OCRL}, a well-established optogenetic version of the inositol 5-phosphatase region of Lowe's oculocerebrorenal protein, OCRL (FIG. 26A). TTRFM studies with iRFP-PH_{PLCδ1} showed that photoactivation of CRY2-5P_{OCRL} evoked rapid redistribution of the PI(4,5)P₂-biosensor away from the cell membrane, with total depletion occurring within ˜225 s (FIGS. 26B, 23C). These findings are consistent with the data we obtained using CRY2-PJ and prior reports on CRY2-5P_{OCRL}.

Like CRY2-PJ, photoactivation of CRY2-5P_{OCRL} caused Nav1.4 currents to activate ˜5 mV earlier than control cells and augmented the peak current-density by ˜10% (FIG. 26D). This effect translated to a 7.5±3 mV left shift in the V½_{act} of Nav1.4 and an augmented the window current (FIG. 27A, Table 1 and Table 2). Neither K_{act}, V½_{SSI} or K_{SSI} were altered by CRY2-5P_{OCRL}. As observed for our studies with CRY2-PJ, including 200 μM diC8PI(4,5)P₂ in the recording pipette right shifted V½_{act} by ˜5 mV and precluded the effects of CRY2-5P_{OCRL} on the voltage-dependent gating of Nav1.4 channels (FIG. 27B). The overall excursion in the mean V½_{ACT} between cells treated with 200 μM diC8PI(4,5)P₂ and control cells recorded following photoactivation of CRY2-5P_{OCRL} was −12.5±3 mV; 75% of the range observed with CRY2-PJ (FIG. 27C and Table 2).

Recovery from Fast Inactivation of Nav1.4 Channels is Speeded by Dephosphorylation of PI(4,5)P₂.

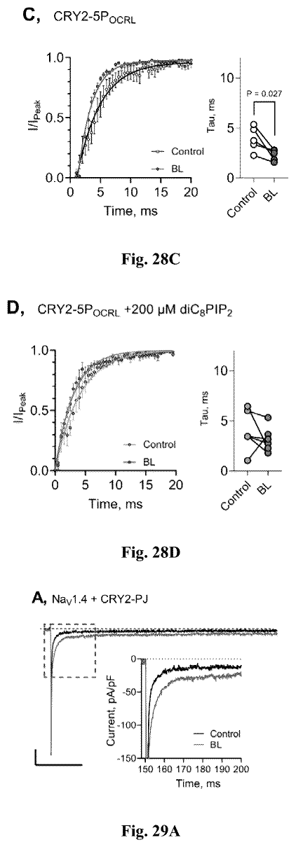

In addition to the voltage-dependence of channel gating, cellular excitability is shaped by the rate at which Nav channels recover from fast inactivation and become available to initiate an action potential. To test if dephosphorylation of PI(4,5)P₂ alters this key gating parameter, we studied the Nav1.4 channels using paired steps to −30 mV and incrementally increased the duration between the pulses. Both dephosphorylation by CRY2-PJ and CRY2-5P_{OCRL} significantly expedited the rate at which Nav1.4 channels recovered from fast inactivation (τ_{recovery}), compared to the control (FIGS. 28A and 28C, respectively). Including 200 μM diC8PI(4,5)P₂ in the pipette precluded the speeding of τ_{recovery} observed when either PI-phosphatase was activated (FIGS. 28B and 28D, respectively). Together these data indicate that the presence of PI(4,5)P₂ in the cell membrane is necessary to maintain the physiological rate at which Nav1.4 channels recover from fast inactivation.

Dephosphorylating PI(4,5)P₂ Slows Fast Inactivation and Augments I_{LATE}.

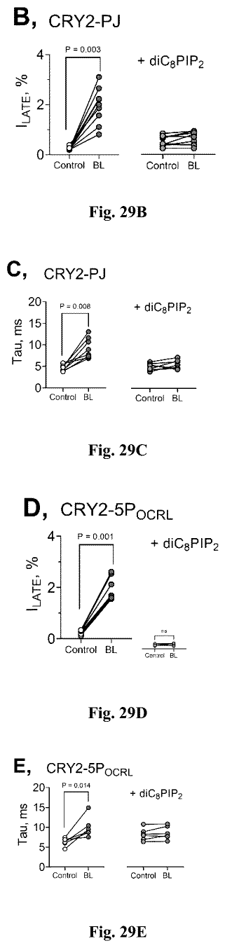

Our study of voltage-dependent activation and steady state inactivation gating revealed that dephosphorylation of PI(4,5)P₂ increased the magnitude of the Nav1.4 window current (FIGS. 25A-inset; 27 Å-inset), suggesting an increased probability of channels remaining in the open state at hyperpolarized voltages. Such changes to the biophysical properties of an Nav channel are expected to manifest an increased I_{LATE} that persists following fast inactivation of the peak current. To study the effect of PI(4,5)P₂ dephosphorylation on I_{LATE}, we recorded currents evoked by a 400 ms depolarizing pulse to −20 mV before and after illumination with blue-light. Photoactivation of CRY2-PJ augmented I_{LATE} by 8-fold from control levels of 0.24% to ˜2% of the peak current (FIGS. 29A, 29B and Table 3). We also assessed the rate of fast inactivation of the peak current by fitting the data to a Boltzmann function and extracting tau (τ_{inact}) values and determined that photoactivation of CRY2-PJ prolonged τ_{inact} from 4.7±0.3 ms to 9±1 ms (FIG. 29C and Table 3). Photoactivation of CRY2-5P_{OCRL} also evoked an 8-fold increase in I_{LATE} and a significant prolongation of τ_{inact} (FIG. 29C and Table 3). Including 200 μM diC₈PI(4,5)P₂ in the recording pipette ameliorated the impact of both CRY2-PJ and CRY2-5P_{OCRL} on I_{LATE} and τinact (FIGS. 29C and 29E, and Table 3). These findings suggest that intact PI(4,5)P₂ modulates the rate and extent of the fast inactivation gating process that transitions the channels from the open to the inactive state.

### PI(4,5)P₂-Dependent Activation Gating Requires the Fast Inactivation Process.

Because of the mechanical linkage between the 4-consecutive domains of Nav channel α-subunits, changes in fast inactivation can also influence the activation process. Therefore, we studied the effects of PI(4,5)P₂ dephosphorylation on an Nav1.4 mutant that abolishes fast inactivation. Nav1.4-L435W-L437C-A438W (Nav1.4-WCW) shows robust expression in Xenopus laevis oocytes and has been reported to disrupt the transitions from activation to inactivation. We expressed Nav1.4-WCW and Nav1β, CIBN-CAAX and CRY2-5P_{OCRL}, with or without the 4-phosphatase CRY2-Sac2, as indicated and studied the effect of PI(4,5)P₂ on the currents in oocytes. Here, we co-express CRY2-5P_{OCRL} and CRY2-Sac2 to generate PI, like with CRY2-PJ in mammalian cells. Nav1.4-WCW passed non-inactivating, voltage-dependent currents that reached a peak at ˜−10 mV (FIG. 30A). CRY2-5P_{OCRL} and CRY2-Sac2 did not impact the kinetics, magnitude, or the V½_{ACT} of the current (FIGS. 30A, 30B). Similarly, photoactivation of CRY2-5P_{OCRL} studied alone did not change the activity of Nav1.4-WCW (FIG. 30C). Together, these data suggest that the impact of PI(4,5)P₂ on the voltage-dependence of Nav1.4 activation requires mechanical coupling to the fast inactivation process and that depleting PI(4,5)P₂ uncouples these key gating processes.

TABLE 1
Na_{V}1.4 channel current-densities before
and after dephosphorylation of PI(4,5)P₂

| Condition | | | |
|---|---|---|---|
| I_{PEAK}, pA/pF | −60 mV | −40 mV | −20 mV |
| CRY2-PJ | | | |
| Control | −10 ± 1 | −262 ± 26 | −280 ± 28 |
| BL | −39 ± 8 | −348 ± 35 | −268 ± 32 |
| | (P = 0.007) | (P = 0.028) | |
| Control + diC₈PI(4,5)P₂ | −19 ± 2 | −194 ± 24 | −264 ± 18 |
| BL + diC₈PI(4,5)P₂ | −23 ± 3 | −209 ± 22 | −272 ± 24 |
| CRY2-5P_{OCRL} | | | |
| Control | −10 ± 4 | −189 ± 24 | −262 ± 24 |
| BL | −37 ± 4 | −305 ± 15 | −247 ± 20 |
| | (P = 0.012) | (P = 0.031) | |
| Control + diC₈PI(4,5)P₂ | −23 ± 6 | −186 ± 28 | −274 ± 32 |
| BL + diC₈PI(4,5)P₂ | −19 ± 4 | −202 ± 21 | −268 ± 25 |

Data are mean current-densities (±s.e.m) obtained from whole-cell patch-clamp recordings, using the current-voltage relationship described in the Materials and Methods and shown in FIGS. 24 and 26. 200 μM diC₈PI(4,5)P₂ was included in the patch pipette when indicated. The means values are from 12-18 cells per group obtained from 3 independent biological replicates of the experiment. Statistical significance was determined using a Student's paired t-test.

TABLE 2
Voltage-dependent activation and inactivation
parameters for Na_{V}1.4 channels

| | G-V | | SSI | |
|---|---|---|---|---|
| | V½_{act}, | K_{Act}, | V½_{SSI}, | K_{SSI}, |
| Condition | mV | mV | mV | mV |

CRY2-PJ

| | | | | |
|---|---|---|---|---|
| Control | −38 ± 2 | 5.0 ± 0.5 | −58 ± 1 | −4.4 ± 0.5 |
| BL | −48 ± 2 | 5.1 ± 0.5 | −58 ± 4 | −7.0 ± 0.6 |
| | (P = 0.007) | | | (P = 0.022) |
| Control + | −33 ± 3 | 4.0 ± 0.7 | −55 ± 4 | −4.2 ± 1.0 |
| diC₈PI(4,5)P₂ | | | | |
| BL + | −33 ± 4 | 5.0 ± 1.0 | −54 ± 2 | −5.1 ± 0.5 |
| diC₈PI(4,5)P₂ | | | | |

CRY2-5P_{OCRL}

| | | | | |
|---|---|---|---|---|
| Control | −37 ± 3 | 5.4 ± 0.4 | −57 ± 3 | −4.7 ± 1 |
| BL | −44 ± 3 | 5.1 ± 0.7 | −58 ± 5 | −6.0 ± 0.7 |
| | (P = 0.009) | | | |
| Control + | −32 ± 1 | 5.0 ± 0.2 | −58 ± 2 | −5.0 ± 2 |
| diC₈PI(4,5)P₂ | | | | |
| BL + | −32 ± 1 | 5.2 ± 0.3 | −57 ± 1 | −5.1 ± 1 |
| diC₈PI(4,5)P₂ | | | | |

Mean V½ values s.e.m.) are determined from Boltzmann function fit to voltage-dependent activation and steady state inactivation studies for Nav1.4 channels, as described in FIGS. 25 and 27. V½_{act} and K_{act} are determined from G-V relationships calculated as described in the Materials and Methods. V½_{SSI} and K_{SSI} are determined from Boltzmann fits to the steady-state inactivation relationships of Nav1.4 channels. Where indicated, 200 μM diC8P(4,5)P₂ was included in the recording pipette. The means values are from 12-18 cells per group obtained from 3 independent biological replicates of the experiment. Statistical significance was determined using a Student's paired t-test.

TABLE 3
Late current parameters for Na_{V}1.4 channels

| | | |
|---|---|---|
| Condition | I_{LATE}, % | tau, ms |

CRY2-PJ

| | | |
|---|---|---|
| Control | 0.24 ± 0.02 | 4.7 ± 0.3 |
| BL | 1.95 ± 0.2 | 8.9 ± 0.8 |
| | (P = 0.0001) | (P = 0.0014) |
| Control + diC₈PI(4,5)P₂ | 0.37 ± 0.1 | 4.7 ± 0.2 |
| BL + diC₈PI(4,5)P₂ | 0.38 ± 0.03 | 5.1 ± 0.3 |

CRY2-5P_{OCRL}

| | | |
|---|---|---|
| Control | 0.24 ± 0.02 | 6.3 ± 0.2 |
| BL | 1.93 ± 0.07 | 10.0 ± 0.4 |
| | (P = 0.0001) | (P = 0.014) |
| Control + diC₈PI(4,5)P₂ | 0.26 ± 0.02 | 6.0 ± 0.3 |
| BL + diC₈PI(4,5)P₂ | 0.27 ± 0.02 | 7.4 ± 0.6 |

I_{LATE} is the percentage of the peak Na⁺ current (I_{PEAK}) measured as an average between 100-150 ms after the start of a depolarizing pulse to −20 mV, as described in the Materials and Methods. The time-constant for fast inactivation, tau, reports the rate of transition from open (I_{PEAK}) to I_{LATE}. Where indicated, diC8PI(4,5)P₂ was included in the recording pipette at 200 μM. The data are from graphs plotted in FIG. 29. The means values are from 10-15 cells per group obtained from 3 independent biological replicates of the experiment. Statistical significance was determined using a Student's paired t-test.

### Example 6: The Role of PI(4,5)P₂ in the Proarrhythmic Activity of Nav1.5 Channels

In healthy hearts, I_{LATE} is typically less than 0.5% of the magnitude of peak I_{Na} and is passed by a small number of Nav1.5 channels that reopen after fast inactivation. In patients with ischemic heart disease, sudden infant death syndrome, or mutations in SCN5 Å that produces long QT syndrome (LQT3), I_{LATE} can increase to 4-5% of peak I_{Na}. Indeed, increases in I_{LATE} of just 0.3-1% can predispose to sudden cardiac death. A range of disease phenotypes, including LQT3, BrS, conduction block and atrial fibrillation, have been associated with mutations, or polymorphisms in SCN5 Å. Nav1.5 is also subject to regulation by a range of protein partners, post-translational modifications (PTMs), and changes in the cellular microenvironment. Dysregulation of channels can be precipitated by the interaction of these functional modifiers. For example, we showed that acute hypoxia augments I_{LATE} due to SUMOylation, a PTM that causes acute regulation of Nav1.2 in neurons and Nav1.5 in human iPS-derived cardiomyocytes. Further, we showed that hypoxia-induced SUMOylation inhibits I_{K1} in RVCMs because SUMO-reduces the efficacy of PI(4,5)P₂ to activate Kir2.1 channels. We recently found that dephosphorylating PI(4,5)P₂ using CRY2-PJ or CRY2-5ptase augments Nav1.4-I_{LATE}. We show preliminary data to support that PI(4,5)P₂ modulation contributes to the phenotype of specific Nav1.5 mutants and hypoxia-induced SUMO regulation and proposes three-experiments designed to characterize these effects.

DiC8-PI(4,5)P₂ Opposes Increased I_{LATE} in Naᵥ1.5-K1493E Channels.

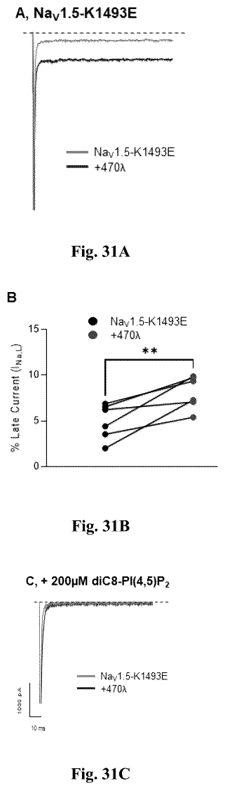

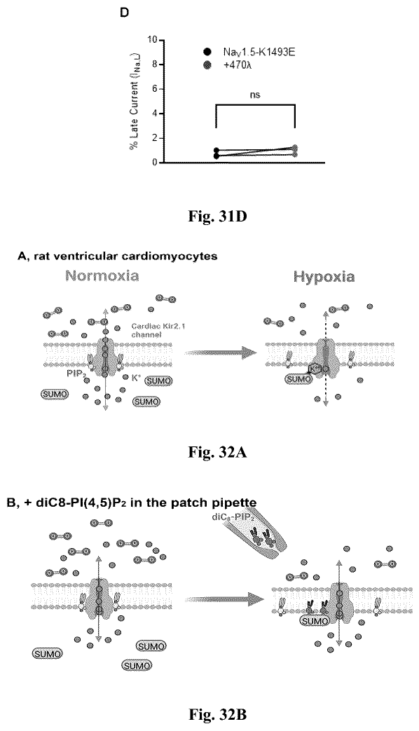

Multiple dysrhythmic disorders are associated with mutations in Nav1.5. Nav1.5-E1784K is the most common variant and is associated with LQT3, BrS, and sinus node dysfunction. E1784K is in the proximal C-terminal domain where it disrupts an interaction with K1493 (DIII-DIV linker) that alters channel gating and increases I_{LATE}. The complementary mutation, K1493E also augments I_{LATE}. We found that including 200 μM diC8-PI(4,5)P₂ in the recording pipette abolished Nav1.5-K1493E I_{LATE} and precluded a further increase in I_{LATE} when we activated CRY2-5ptase (FIG. 31). These findings indicate a potential role for PI(4,5)P₂ in the mechanism of, at least some, Nav1.5 disease mutants.

DiC8-PI(4,5)P₂ Opposes Hypoxia-Induced Increase in I_{LATE} in RVCMs.

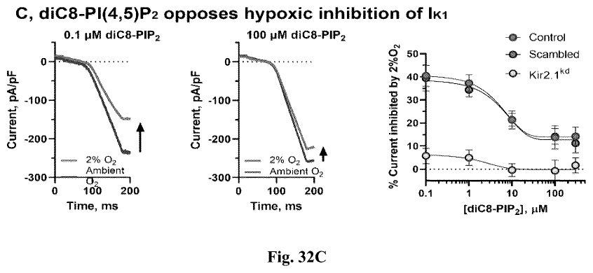

We found that acute hypoxia (1.5% 02) increases Nav1.5-I_{LATE} to pro-arrhythmic levels in iPS-CMs via increased Nav1.5 SUMOylation. We also showed that acute hypoxia inhibits I_{K1} in RVCMs because SUMO interferes with PI(4,5)P₂ activation of Kir2.1 channels (FIGS. 32A-32B). Further, the effects of hypoxia and SUMO on I_{K1} are opposed, in a concentration dependent manner, by adding exogenous diC8-PI(4,5)P₂ in the recording pipette (FIGS. 6B-6C). Further, the hypoxic response is diminished in RVCMs where Kir2.1 has been knocked down by transduction with lentiviral particles carrying eGFP and shRNA targeting kcnj2, the mRNA that produces Kir2.1 (Kir2.1^{kd}, FIG. 32C). These findings demonstrate that PI(4,5)P₂ mediates the effects of SUMO on Kir2.1 channels. However, we do not know if diC8-PI(4,5)P₂ opposes the hypoxia-induced increase in I_{LATE}. Our findings on Kir2.1 lead us to hypothesize that PI(4,5)P₂ might play a role in mediating the effects of hypoxia induced SUMOylation on Nav1.5.

### Experimental Design

Nav1.5 channels initiate the heartbeat, and their dysregulation can precipitate arrhythmias. Our data support that changes in Nav1.5 function associated with at least some mutations and exposure to acute hypoxia might result from perturbation of the relationship between the channel and PI(4,5)P₂. Here, we describe experiments designed to test this idea and characterize the role of PI(4,5)P₂ in the gating of mutant Nav1.5 channels. Although many mutants are clustered on the DIII-DIV linker of the channel, we will focus our initial efforts on studying Nav1.5-E1784K in the proximal C-terminus and Nav1.5-K1493E, the cognate residue in the DIII-DIV linker. With this strategy, we expect to gain a deeper understanding of the role of PI(4,5)P₂ in mediating the phenotypic effects of these mutations and if exogenous diC8-PI(4,5)P₂ can oppose the augmented I_{LATE}.

### Effect of Dephosphorylating PI(4,5)P₂ on Disease Variant Nav1.5 Channels.

Dephosphorylating PI(4,5)P₂ modulates multiple aspects of Nav1.4 channel gating and augments Nav1.5-I_{LATE}. However, it is not known if Nav1.5-channelopathies are sensitive to PI(4,5)P₂ dephosphorylation. To test this, we express Nav1.5-E1784K, Navβ1, CIBN-CAAX and CRY2-PJ (or CRY2-5ptase) in HEK293T and use whole-cell patch-clamp to study the I-V, G-V, SSI, RFI relationships, and I_{LATE} before and after dephosphorylation of PI(4,5)P₂. We perform the same studies with Nav1.5-K1493E and compare the data with those obtained for wild type Nav1.5. Because both mutants have an enhanced I_{LATE}, we perform the studies in the background of Nav1.5-C373F and use TTX at the end of each experiment to baseline the current.

### Can Exogenous PI(4,5)P₂ Oppose the Phenotypic Effects of Disease Variant Nav10.5 Channels?

Our data show that adding 200 μM diC8-PI(4,5)P₂ to the recording pipette abolishes I_{LATE} in Nav1.5-K1493E channels but it is not known if diC8-PI(4,5)P₂ also opposes other phenotypic changes associated with K1493E. To address this question, we study the effects of diC8-PI(4,5)P₂ on the I-V, G-V, SSI and RFI relationships of Nav1.5-E1784K and Nav1.5-K1493E channels. In addition, we fully characterize the effects of diC8-PI(4,5)P₂ on macroscopic I_{LATE} from both mutants. We also study these effects using single channel recording. Here, diC8-PI(4,5)P₂ can be applied to the cytoplasmic face of the mutant channels in off-cell, inside-out patches to establish the concentration dependence of rescue. Further, we test the effect of PI(4)P. Other minor PI species will be studied to test if they also reduce I_{LATE}. Based on our findings, diC8-PI(4,5)P₂ opposes I_{LATE} in Nav1.5-K1493E and Nav1.5-E1784K channels.

Do Hypoxia and SUMOylation Modulate Naᵥ1.5 Via PI(4,5)P₂?

We have shown that acute hypoxia augments I_{LATE} because of rapid SUMOylation of Nav1.5 at K442. However, it is not known if this effect is mediated by PI(4,5)P₂. To answer this question, we evoke an increase in macroscopic I_{LATE}, as before, by switching to a hypoxic (typically 1.5% 02) perfusate that has been bubbled with N2 for at least 30 min prior to the experiment. In such studies, we measure the O₂-tension at the cell using a calibrated O₂-probe (Ocean Insight). We find that solution exchange occurs in less than 20 s via gas impermeable Tygon tubing and have shown that this manipulation increases I_{LATE} from ˜0.46% to ˜4.4% of the peak I_{Na} in human iPS-CMs and inhibits I_{K1} in RVCMs (FIG. 32). We repeat this study with increasing concentrations of diC8-PI(4,5)P₂ or diC8-PI(4)P in the pipette on Nav1.5 channels expressed in HEK293T cells and on native currents in RVCMs. Based on our work, increasing the concentration of diC8-PI(4,5)P₂ opposes the effects of hypoxia. As a control, we study Nav1.5-K442Q, a hypoxia-insensitive variant that lacks the lysine residue needed to link SUMO to the channel.

### INCORPORATION BY REFERENCE

All publications, US patents, and US and PCT published patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating or preventing a cardiovascular disease, a pain syndrome, epilepsy, or a skeletal muscle disease, comprising administering to a subject in need thereof an effective amount of a compound;
wherein the compound increases the interaction between Naᵥ1.5 voltage-gated sodium channel or a Naᵥ1.4 voltage-gated sodium channel and Phosphatidylinositol 4,5-bisphosphate (PIP2),
the compound is an analogue of PIP2;
the compound is PIP2; or
the compound increases endogenous PIP2.

2. The method of claim 1, wherein the compound increases endogenous PIP2; and the compound is an inhibitor of a Gαq-coupled AT1 receptor.

3. The method of claim 2, wherein the inhibitor of a Gαq-coupled AT1 receptor is losartan, Exp 3174, telmisartan, irbesartan, candesartan, valsartan, eprosartan, azilsartan, saprisartan or olmesartan.

4. The method of claim 1, wherein the compound increases endogenous PIP2; and the compound is an inhibitor of phospholipase C (PLCβ).

5. The method of claim 4, wherein the inhibitor of PLCβ is U73122, phenylmethylsulfonyl fluoride, manoalide, D609, ET-18-OCH3, compound 48/80 trihydrochloride, spermine tetrahydrochloride, neomycin sulfate, NCDC, or thielavin B.

6. The method of claim 1, wherein the compound is an analogue of PIP2; and the PIP2 analogue is diC8-PIP2.

7. The method of claim 1, wherein the compound increases endogenous PIP2; and the compound inhibits PIP2 hydrolysis or inhibits PIP2 dephosphorylation.

8. The method of claim 1, wherein a cardiovascular disease is treated or prevented; and the cardiovascular disease is arrhythmias, long QT syndrome (LQT3), Brugada syndrome (BrS), cardiac conduction defects, atrial fibrillation, and dilated cardiomyopathy, sudden infant death syndrome (SIDS), or sudden cardiac death in adults.

9. The method of claim 1, wherein a pain syndrome is treated or prevented; and the pain syndrome is a chronic pain syndrome, fibromyalgia, or neuropathic pain.

10. The method of claim 1, wherein epilepsy is treated or prevented; and the epilepsy is an idiopathic generalized epilepsy, idiopathic partial epilepsy, symptomatic generalized epilepsy or symptomatic partial epilepsy.

11. The method of claim 10, wherein the symptomatic partial epilepsy is temporal lobe epilepsy.

12. The method of claim 10, wherein the subject suffers from a traumatic brain injury, hypoxic brain injury, brain infection, stroke, or genetic syndrome.

13. The method of claim 12, wherein the subject suffers from a brain infection; and the brain infection is encephalitis, meningitis, mesial temporal sclerosis, or a cerebral tumor.

14. The method of claim 12, wherein the epilepsy is at least partially induced by the traumatic brain injury, hypoxic brain injury, brain infection, stroke, or genetic syndrome.

15. The method of claim 10, wherein the epilepsy is a TBI-induced epilepsy.

16. The method of claim 1, wherein the subject suffers from a skeletal muscle disease; and the skeletal muscle disease is a periodic paralysis (PP), a nondystrophic myotonia (NDM), and a ryanodinopathy.

17. The method of claim 16, wherein the periodic paralysis (PP) or the nondystrophic myotonia (NDM) is myotonia congenita, paramyotonia congenita (PMC), or potassium-aggravated myotonia (PAM), hyper and hypokalemic periodic paralysis (hyperPP and hypoPP), or Andersen-Tawil syndrome (ATS).

18. The method of claim 16, wherein the ryanodinopathy is malignant hyperthermia (MH), central core disease (CCD), multi-minicore disease (MmD), or centronuclear myopathy (CNM).

19. A method of identifying a compound that modulates the interaction between a Na₁1.5 voltage-gated sodium channel or a NaV1.4 voltage-gated sodium channel and PIP2, comprising:
contacting cells expressing a tagged NaV1.5 channel, a tagged NaV1.4 channel, or a variant thereof, with a candidate agent and PIP2 or a PIP2 analog; or contacting cell lysate of cells expressing a tagged NaV1.5 channel, a tagged NaV1.4 channel, or a variant thereof, with a candidate agent and PIP2 or a PIP2 analog;
detecting the affinity of PIP2 or the PIP2 analog to the tagged NaV1.5 channel or the tagged NaV1.4 channel,
comparing the affinity in the presence of the candidate agent with the affinity in the absence of the candidate agent; wherein a change in affinity in the presence of the candidate agent is indicative of modulation of the interaction between NaV1.5 or NaV1.4 and PIP2.

20. The method of claim 19, wherein the tagged NaV1.5 channel, the tagged NaV1.4 channel, or a variant thereof is isolated from the cell lysate before incubating with the candidate agent and PIP2 or the PIP2 analog.

21-25. (canceled)
